(19) United States
(12) Patent Application Publication
(10) Pub. No.: US 2015/0259674 A1
(43) Pub. Date: Sep. 17, 2015

(54) COMPOSITIONS AND METHODS FOR LONG INSERT, PAIRED END LIBRARIES OF NUCLEIC ACIDS IN EMULSION DROPLETS

(71) Applicant: The Broad Institute, Inc., Cambridge, MA (US)

(72) Inventors: Scott Steelman, Wilmington, MA (US); Robert Nicol, Cambridge, MA (US); Robert Lintner, Amesbury, MA (US)

(21) Appl. No.: 14/664,331

(22) Filed: Mar. 20, 2015

Related U.S. Application Data
(63) Continuation-in-part of application No. PCT/US2013/061182, filed on Sep. 23, 2013.
(60) Provisional application No. 61/703,884, filed on Sep. 21, 2012.
(60) Provisional application No. 61/731,021, filed on Nov. 29, 2012.
(60) Provisional application No. 61/779,964, filed on Mar. 13, 2013.

(51) Int. Cl. C12N 15/10

(52) U.S. Cl. CPC C12N 15/1065; C12N 15/1075

(57) ABSTRACT

The invention provides for methods for uniquely labeling populations of nucleic acids of interest in emulsion droplets using random combinations of oligonucleotides. The labeling methodology of the invention may be used, inter alia, to generate mate pair genomic fragments without the need for circularization. Because the method is independent of circularization, mate pairs can be generated from any length genomic fragment.

### RELATED APPLICATIONS AND INCORPORATION BY REFERENCE

This application is a continuation-in-part application of international patent application Serial No. PCT/US2013/061182 filed Sep. 23, 2013, which published as PCT Publication No. WO 2014/047556 on Mar. 27, 2014, which claims the benefit of and priority to U.S. Provisional Application Ser. No. 61/779,964, filed Mar. 13, 2013; 61/731,021, filed Nov. 29, 2012; and 61/703,884, filed Sep. 21, 2012. This application is related to U.S. Provisional Application Ser. No. 61/779,999, filed Mar. 13, 2013.

The foregoing application, and all documents cited therein (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The invention is directed to methods for uniquely labeling populations of nucleic acids of interest in emulsion droplets using random combinations of oligonucleotides.

### BACKGROUND OF INVENTION

The ability to label and create mate-pair libraries serves multiple purposes in research and industry. However, several limitations exist in current labeling technologies. These limitations include the limited number of distinguishable detectable moieties currently in existence, the amount of time required to uniquely label a plurality of library elements, the requirement, in some instances, for specialized equipment, and the cost involved.

### SUMMARY OF INVENTION

The invention provides methods and compositions for uniquely labeling nucleic acids, such as DNA, peptides or proteins. One of the major limitations of prior art labeling techniques is the limited number of available unique labels. Typically the number of nucleic acids to be labeled in any given application far exceeds the number of unique labels that are available. The methods of the invention can be used to synthesize essentially an infinite number of unique labels. Moreover, because of their nature, the labels can be easily detected and distinguished from each other, making them suitable for many applications and uses. The methods of the invention also provides for amplifying nucleic acids to increase the number of read pairs properly mated via their unique index combination. Additionally, the methods of the invention allow for each end-labeled nucleic acid to be identically labeled at its 5′ and 3′ ends.

In one embodiment, provided is a method for labeling a nucleic acid at both its 5′ and 3′ ends with a unique label, which may comprise the steps of:
a) providing a pool of nucleic acids; and
b) sequentially end-labeling said nucleic acids with a random combination of n detectable oligonucleotide tags, each of said oligonucleotide tags optionally which may comprise a cohesive overhang of x base pairs in length, wherein each detectable oligonucleotide tag is randomly and independently selected from a number of detectable oligonucleotide tags that is less than the number of nucleic acids, and n is the number of oligonucleotides attached to an end of said nucleic acid,
wherein said method is performed in emulsion droplets, and wherein each end-labeled nucleic acid is identically labeled at its 5′ and 3′ ends.

Also provided are labeled nucleic acids and libraries of said nucleic acids.

### BRIEF DESCRIPTION OF DRAWINGS

Non-limiting embodiments of the present invention will be described by way of example with reference to the accompanying Figures, which are schematic and are not intended to be drawn to scale. For purposes of clarity, not every component is labeled in every figure, nor is every component of each embodiment of the invention shown where illustration is not necessary to allow those of ordinary skill in the art to understand the invention.

FIG. 1 shows that the efficiency of DNA circularization (cyclization) decreases as fragment length increases.

FIG. 2 is a schematic depicting the technique for polymerase mediated index addition.

FIG. 3 is a gel electrophoresis showing ligation of an adapter sequence in either a tube (T) or an emulsion droplet (E).

FIG. 4 is a schematic depicting the technique for ligation mediated index addition.

FIG. 5 is a schematic depicting the technique for symmetric ligation-mediated index addition.

FIG. 6 is a flowchart detailing the criteria for barcode sequence selection.

FIG. 7 is a schematic depicting the methodology for informatically deriving mate pairs.

FIG. 8 shows catalyzing ligation by the controlled addition of MgCl₂.

FIG. 9 shows a stability determination of MgCl₂ in droplets.

FIG. 10 shows a determination of the optimal ratio of genomic Index:genomic DNA.

FIG. 11 is a schematic depicting the process of symmetric indexing in emulsion.

FIG. 12 shows a proof of concept experiment.

FIG. 13 shows an analysis of E. coli proof of concept libraries.

FIG. 14 shows an analysis of lambda proof of concept libraries.

FIG. 15 shows a determination of symmetry of indexing in E. coli proof of concept libraries.

FIG. 16 is a schematic of a mate pair synthesis process using single stranded genomic DNA as the agent.

FIG. 17 is a schematic of a mate pair synthesis using droplets and Nextera transposomes as detectable tags.

FIG. 18 shows the determination of uniformity of blunt-ended indexing.

FIG. 19 shows impact of ligation efficiency on bioinformatics end association.

FIG. 20 shows a redesign of index sequences.

FIG. 21 shows uniformity of indexing.

FIG. 22 shows amplification of fragment ends via transposome-based selection.

FIGS. 23a and 23b illustrate enrichment of ends via in vitro transcription.

FIG. 24 shows amplification of ends via anchored PCR.

FIG. 25 depicts creating multi-kilobase fragment reads from indexed DNA ends.

### DETAILED DESCRIPTION OF INVENTION

The methods of the invention easily and efficiently generate libraries of unique labels. Such libraries may be of any size, and are preferably large libraries including hundreds of thousands to billions of unique labels. The libraries of unique labels may be synthesized separately or may be synthesized in real-time (e.g., while in the presence of the nucleic acid). Methods for nucleic acid sequencing and detection of non-nucleic acid detectable moieties are known in the art and are described herein.

The methods of the invention label nucleic acids in emulsion droplets. The nucleic acid is identically labeled at its 5′ and 3′ ends. Further, the nucleic acids are amplified by a method such as, for example, anchored PCR.

The invention further provides for methods for creating mate-pair libraries of uniquely labeled nucleic acids such as genomic DNA fragments. The term mate-pair, although specific to certain next generation sequencing technologies, is intended to generically describe a “jumping” library. A jumping library is any DNA construct where the physical genomic distance between sequencing reads can be derived without the need to sequence the entire intervening length of DNA. Depending on the application and/or sequencing technology, some terms frequently used to describe such libraries include (but are not limited to): jumping libraries, distance libraries, long range libraries, linking libraries, long distance linking libraries, mate-pair libraries and long paired-end libraries.

The invention contemplates that the labels are prepared by sequentially attaching randomly selected oligonucleotides (referred to interchangeably as oligonucleotide tags) to each other. The order in which the oligonucleotides attach to each other is random and in this way the resultant label is unique from other labels so generated. The invention is based, in part, on the appreciation by the inventors that a limited number of oligonucleotides can be used to generate a much larger number of unique labels. The invention therefore allows a large number of labels to be generated (and thus a large number of nucleic acids to be uniquely labeled) using a relatively small number of oligonucleotides.

The unique labeling strategies of the invention provide methods for generating mate pairs from genomic DNA fragments of virtually any length. This is a significant advantage over the mate pair methods of the prior art which require circularization of the genomic DNA fragment and thus are limited by the length of the fragment. In contrast, the methods of the invention do not rely on circularization of the genomic DNA fragments and thus are able to generate mate pairs from genomic DNA fragments of various lengths.

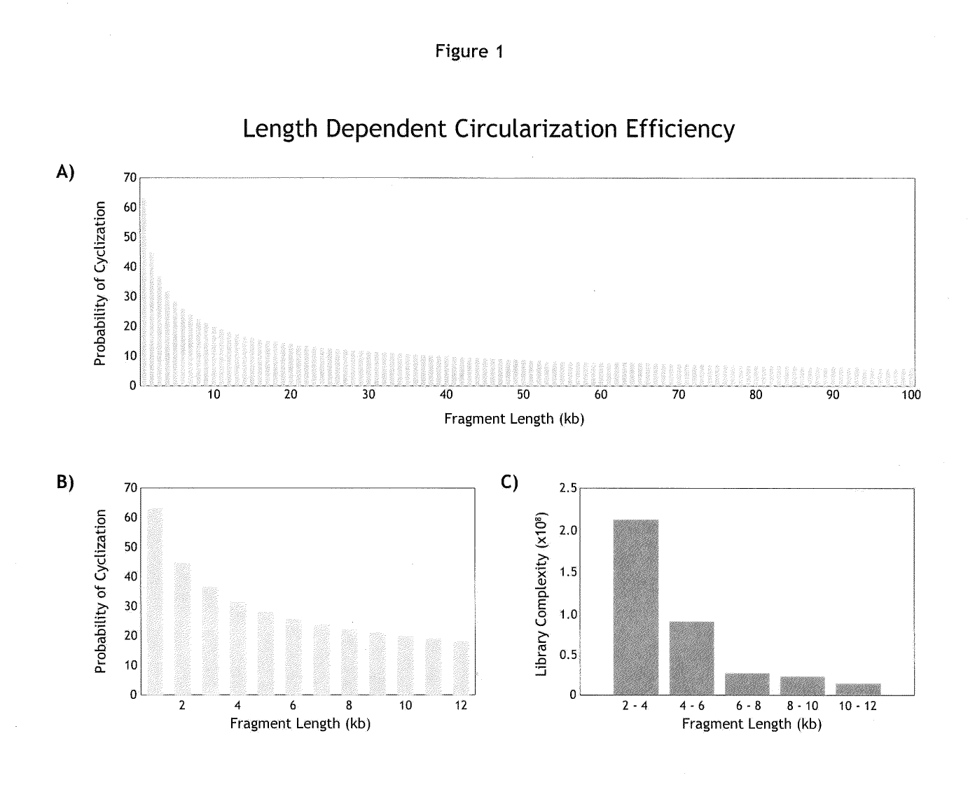

Certain aspects of the invention, and the advantages of the invention over previous techniques, are shown in the Figures. FIG. 1 shows that the efficiency of DNA circularization (cyclization) decreases as fragment length increases. A) Predicted probability of circularization for DNA fragments ranging from 1 kb to 100 kb. Probability values are derived using the Jacobson-Stockmayer factor, j=(3/2pbl)3/2 (where l=contour length and b=hydrodynamic segment length), which is a probability density function that describes the effective concentration of one end in the neighborhood of the other end on the same molecule of a random coil polymer. B) Portion of the graph presented in panel A focused on the 1 kb to 12 kb size range. C) Illumina mate-pair libraries were prepared from human genomic DNA fragments of the sizes indicated. Estimated jumping library complexity (shown on the Y-axis) decreases rapidly as fragment size increases due largely to inefficiencies at circularization.

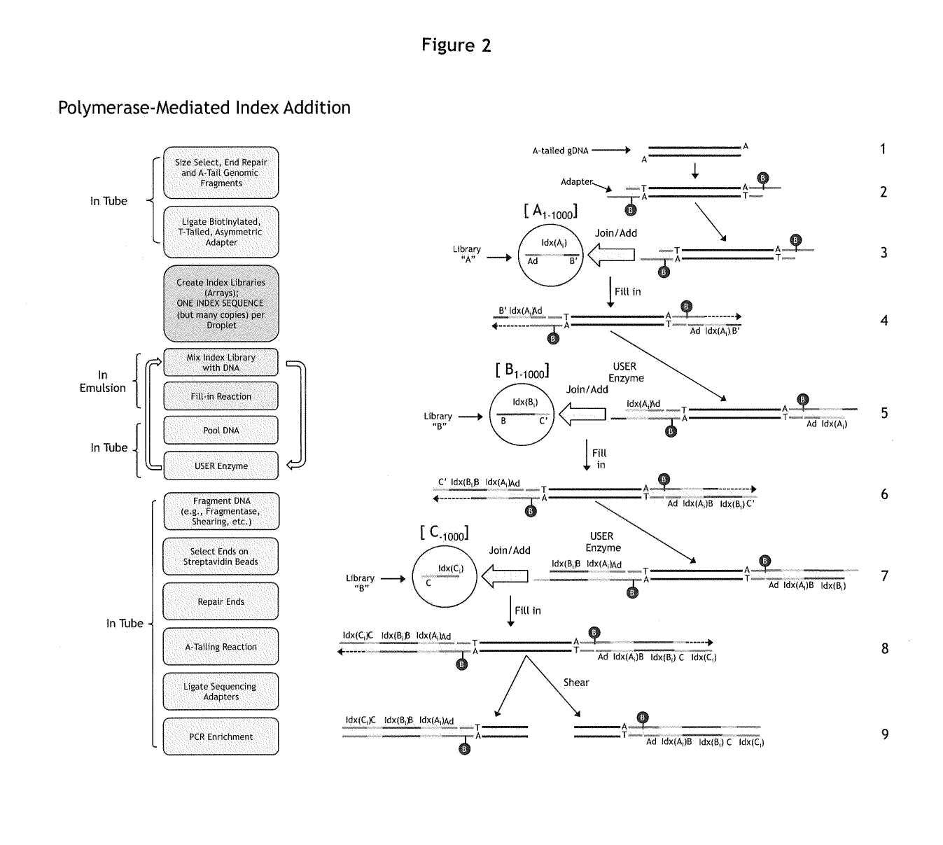

FIG. 2 shows the technique for polymerase mediated index addition. As seen in FIG. 2, (1) genomic DNA is size selected, end repaired and A-tailed. (2) The biotinylated adapter can only ligate in one orientation on each end of the genomic DNA due to the presence of the T-tail. The adapter is a partial duplex to allow for primer annealing in the next step of the process. (3) Multiple index libraries are created in emulsion and a polymerase-driven fill-in reaction is used to add the index. The index libraries may contain some or all of the key components of the fill in reaction (i.e., MgCl₂, dNTP, polymerase). The library may be comprised of unique single stranded DNA oligonucleotides (oligos). Each oligo will contain 3 distinct moieties: sequence complimentary to adapter (Ad), a unique index sequence (Idx) and a sequence used to “capture” the next index oligo which contains one or more dUTP nucleotides (B′/C′). DNA is diluted to a desired concentration to control the number of molecules per droplet and is merged with the index droplet library. (4) A fill-in reaction is performed creating the complement to the index and the “capture” site. (5) The emulsion is broken and DNA is pooled prior to treatment with USER enzyme causing the “capture” portion of the indexed oligo to be digested/released from the nascent strand which does NOT contain dUTP. (6-8) The process of DNA dilution, index addition, fill-in, pool, and USER enzyme treatment is repeated for the desired number of cycles, each time adding one new index to the end of the DNA. (9) After the final index addition, DNA is fragmented and the ends are collected via streptavidin beads. All fragments are ligated to technology specific sequencing adapters and ends are informatically paired based on their unique string of indexes.

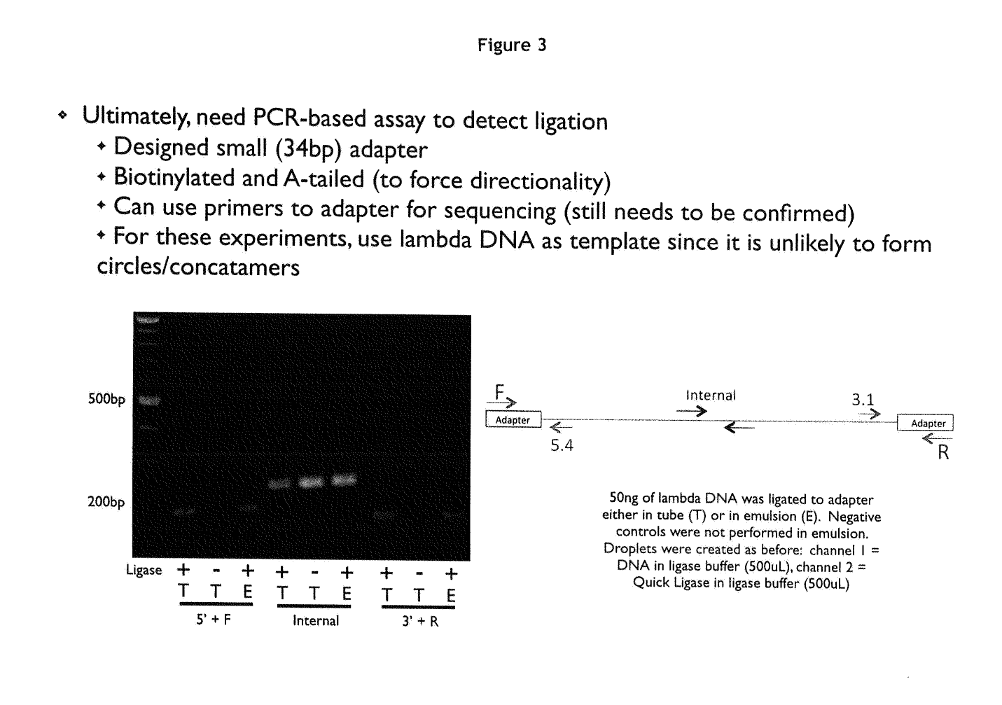

FIG. 3 is a gel electrophoresis showing ligation of an adapter sequence in either a tube (T) or an emulsion droplet (E).

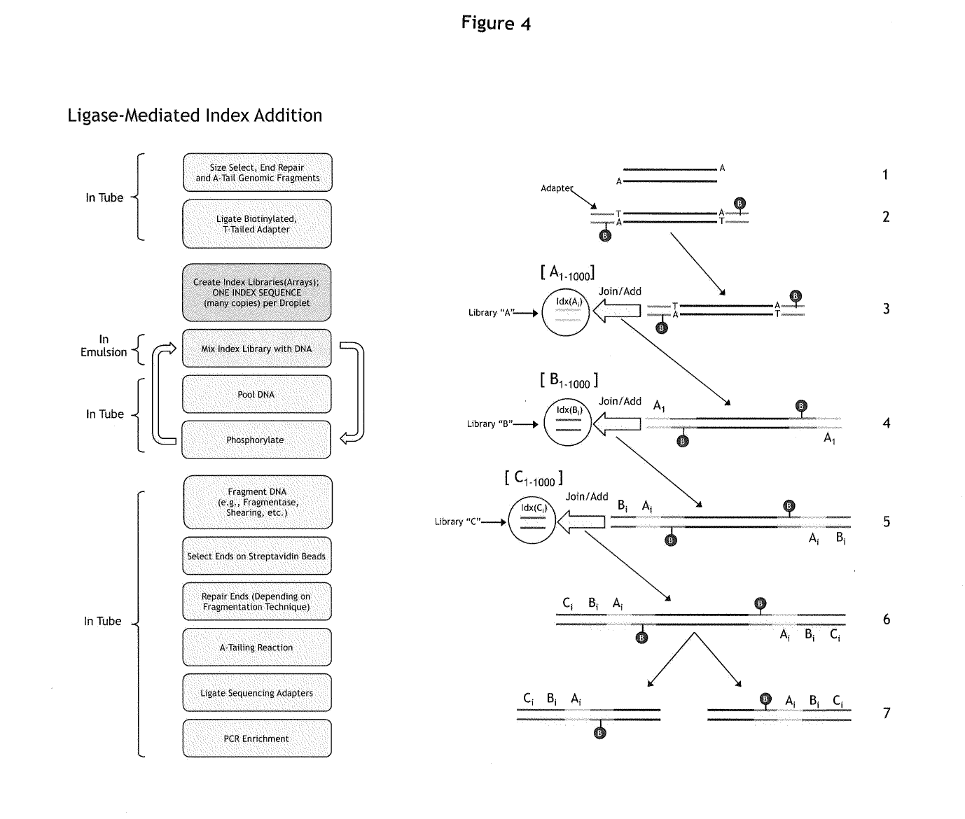

FIG. 4 is a schematic of the technique for ligation mediated index addition. As shown in FIG. 4. (1) Genomic DNA is size selected, end repaired and A-tailed. (2) The biotinylated adapter can only ligate in one orientation on each end of the genomic DNA due to the presence of the T-tail. The adapter may be blunt ended or it may be a partial duplex with a sequence specific cohesive overhang to allow for index annealing in the next step of the process. (3) Multiple index libraries are created in emulsion and a ligation reaction is performed to add the index. The index libraries may contain some or all of the key components of the ligation reaction (i.e., MgCl₂, dNTP, ligase). Each droplet will contain many copies of an individual (unique) index sequence. DNA is diluted to a desired concentration to control the number of genomic fragments per droplet and is then joined to the index library allowing the ligation reaction to occur. Following ligation, the emulsion is broken and DNA is pooled, purified and prepared so that it can accept the next index. (4-6) The process of DNA dilution, index addition, ligation, pooling, clean-up and phosphorylation is repeated for the desired number of cycles, each time adding one new index to the end of the DNA. (7) After the final index addition, DNA is fragmented and the ends are collected via streptavidin beads. All fragments are ligated to technology specific sequencing adapters and ends are informatically paired based on their unique string of indexes.

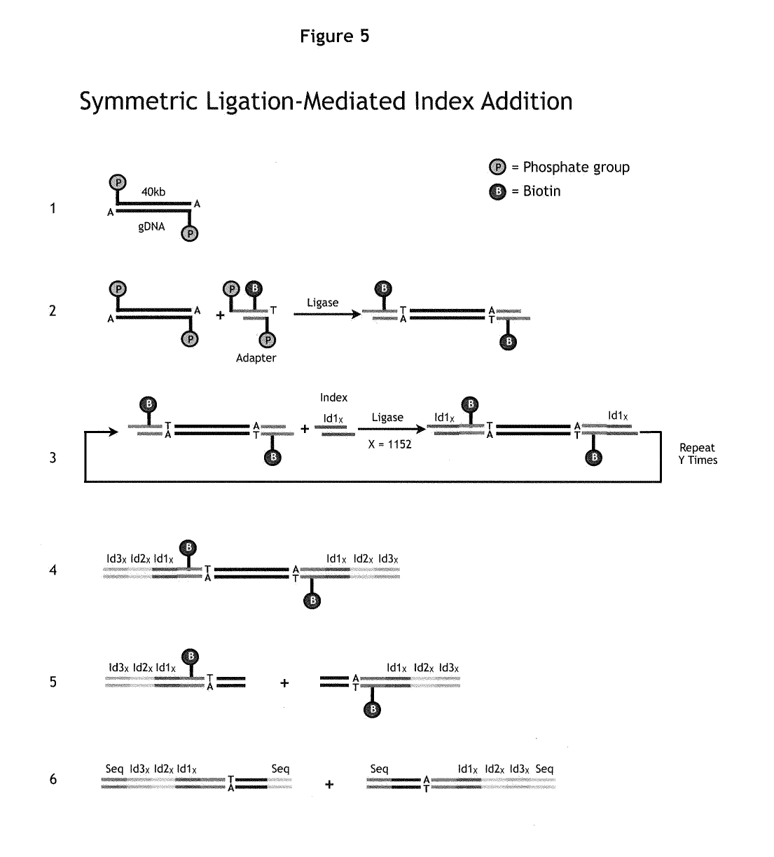

FIG. 5 depicts the technique for symmetric ligation-mediated index addition. (1) Genomic DNA is size selected, end repaired and A-tailed. (2) The biotinylated adapter can only ligate in one orientation on each end of the genomic DNA due to the presence of the T-tail. The adapter may be blunt ended or it may be a partial duplex with a sequence specific cohesive overhang to allow for index annealing in the next step of the process. (3) Multiple index libraries are created and a ligation reaction is performed to add the index. Following ligation, DNA molecules are pooled, purified and prepared for the next round of ligation. (4) This process is repeated Y number of times. The total “diversity” of the population of unique index combinations is dictated by the number of indexes used raised to the power of the number of cycles performed. For example, 3 round of index ligation using a 1152 element array creates 1152³ or 1,528,823,808 combinations. (5) After the final index addition, DNA is fragmented and the ends are collected via streptavidin beads. (6) Sheared DNA fragments are end repaired as needed and ligated to technology specific sequencing adapters. Following sequencing, the ends are informatically paired based on their unique string of indexes.

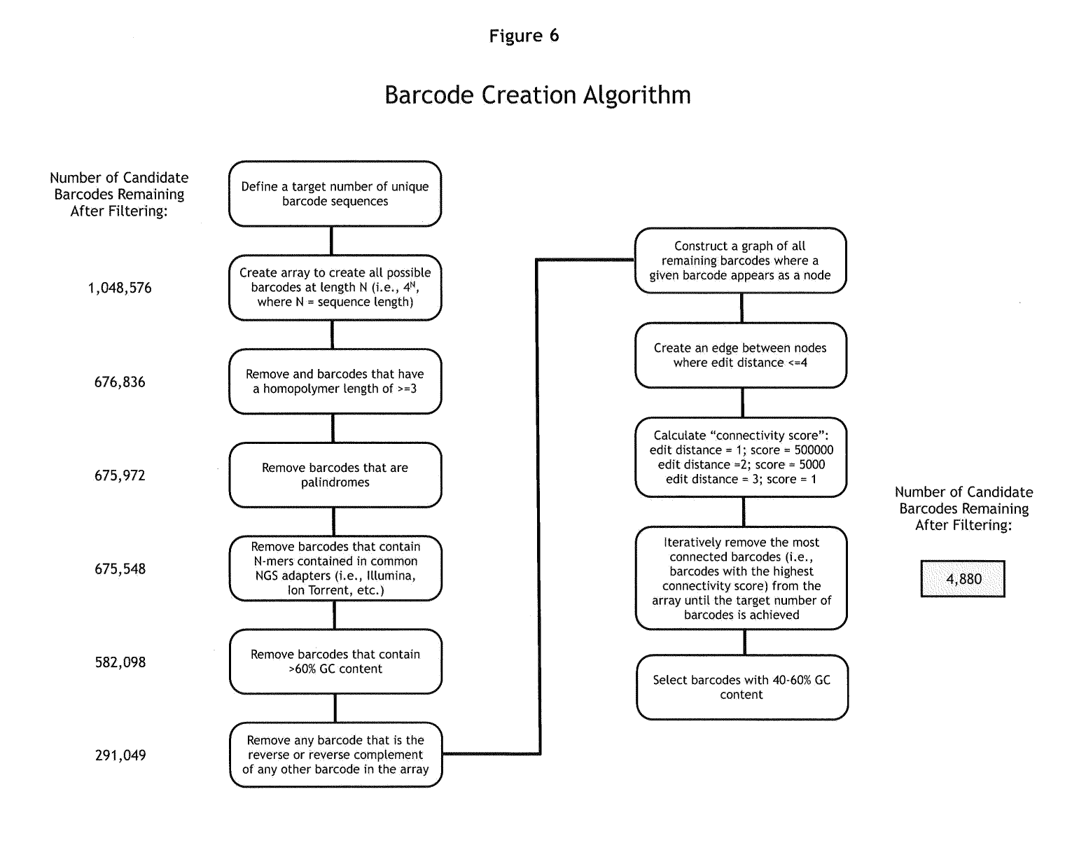

FIG. 6 is a flowchart detailing the criteria for barcode sequence selection.

Though the set started with a hypothetical pool of >1 million random sequences, the filtering criteria eliminated over 99% of the random sequences to arrive at a collection of ˜5,000 barcode sequences.

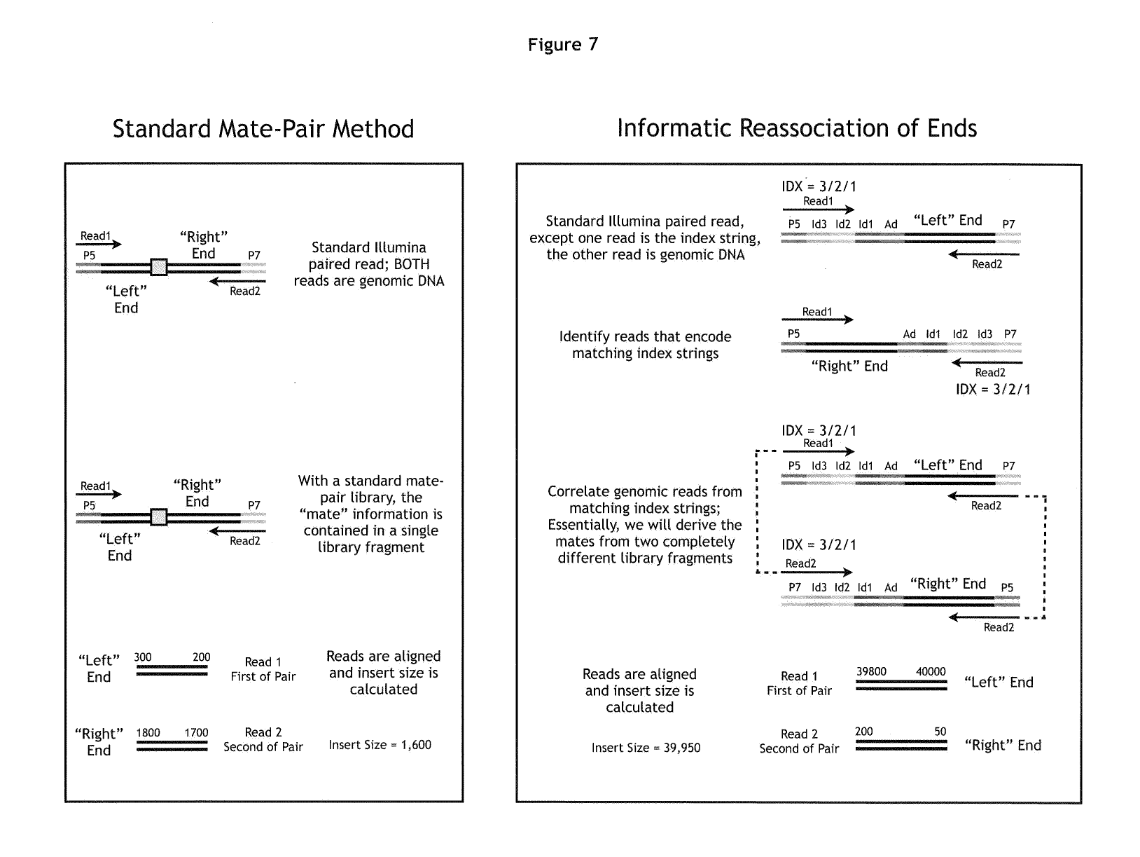

FIG. 7 provides the methodology for informatically deriving mate pairs. Left Panel: For a standard Illumina mate-pair library, each construct is sequenced using two reads. Since both ends of the parent genomic DNA fragment are contained in a single construct, two reads (i.e., a single read pair) are sufficient to establish a mate-pair. Right Panel: Unlike a standard library, a symmetrically indexed library requires a total of 4 reads (i.e., 2 read pairs) in order to establish a mate-pair. For a given construct, one read will contain the index information while the other is genomic DNA (i.e, one read pair defines one “half” of the mate-pair). For each construct in the library, the algorithm must search the data set to identify appropriately matching indexing combinations. Once the index combinations are matched, their corresponding genomic reads can be positioned relative to the genome.

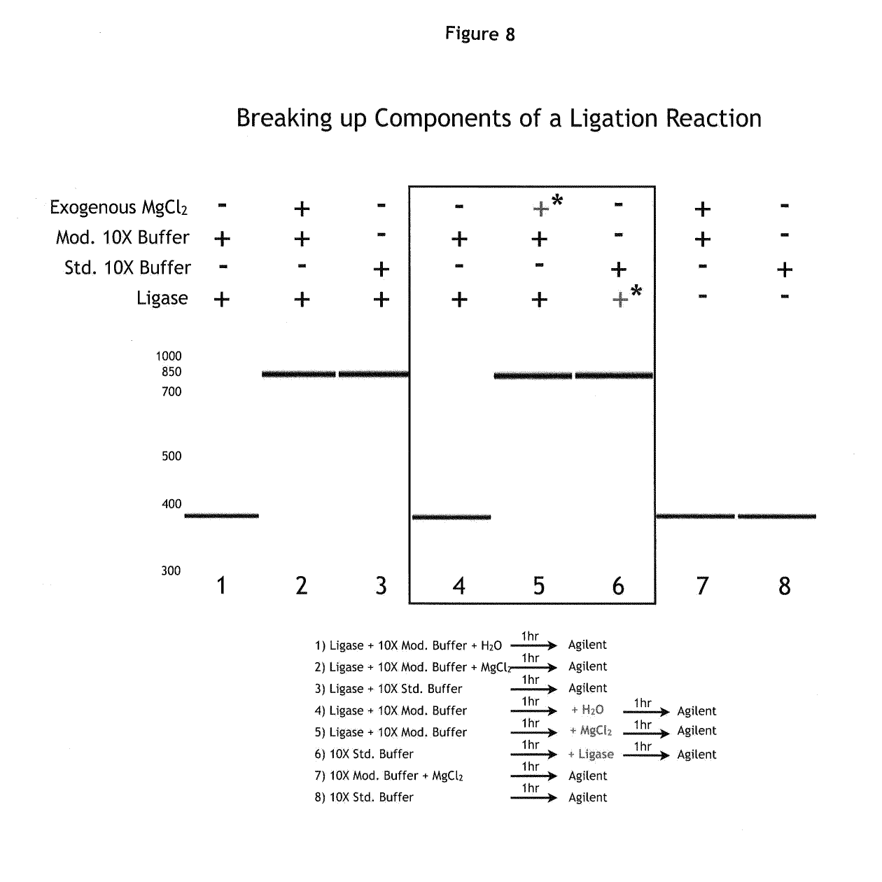

FIG. 8 shows catalyzing ligation by the controlled addition of MgCl₂. In order to prevent spontaneous ligation (concatamerization) of genomic DNA fragments, it is necessary to prepare the DNA in a solution that lacks one or more of the key components necessary to catalyze a ligation reaction. Due to stability and cost issues, sequestration of MgCl₂ in the index droplets would be preferable over other key components (i.e., ligase enzyme and ATP). A single 380 bp restriction fragment was generated from pBR322 plasmid DNA and prepared it such that it can only ligate to itself in one orientation. Thus, a 760 bp ligated product is easily distinguishable from the unligated 380 bp product. A 10× modified ligation buffer lacking MgCl₂ was prepared but containing 500 mM Tris-HCl pH 7.5, 100 mM dithiothreitol and 10 mM ATP. For lanes 1-3 and lanes 7 and 8, reactions were prepared as indicated, incubated for 1 hour at room temperature, purified and run on an Agilent DNA 1000 chip. For lanes 4, 5 and 6, reactions were prepared as indicated and incubated for 1 hour at room temperature. Following this initial incubation, either water (lane 4), MgCl₂ (lane 5) or T4 DNA ligase (lane 6) were added to the reaction. Samples were then allowed to incubate for an additional 1 hour at room temperature before being purified and run on an Agilent DNA 1000 chip. No ligated product was observed in those reactions lacking MgCl₂ (lanes 1 and 4). Upon addition of 50 mM MgCl₂ at either time=0 (lane 2) or time=1 hour (lane 5), ligated product was observed.

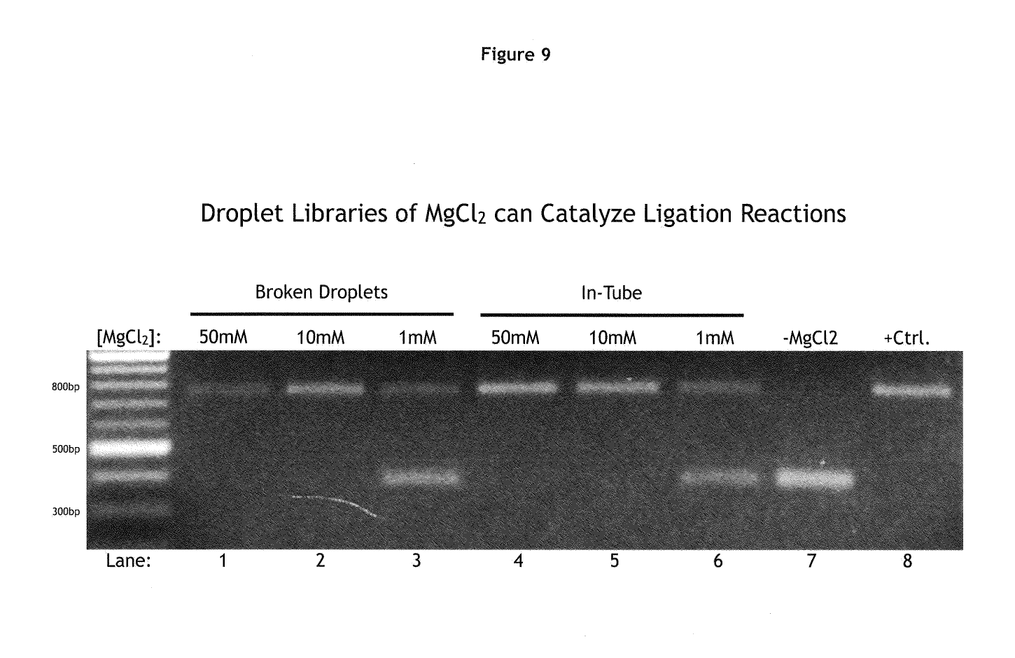

FIG. 9 shows stability determination of MgCl₂ in droplets. Droplets containing 50 mM, 10 mM or 1 mM concentrations of MgCl₂ were prepared and stored at +4° C. for ˜4.5 days. Droplets were then broken and the aqueous phase was collected from each droplet “library” and transferred into fresh 1.5 ml tubes. Ligation reactions containing IX Modified Ligation buffer (i.e., buffer lacking MgCl₂), T4 DNA ligase and 380 bp control fragment DNA were prepared. Aqueous phase recovered from the various droplet libraries (lanes 1-3) or non-emulsified MgCl₂ (lanes 4-6) was added to the various ligation reactions. The 50 mM and 10 mM reactions appeared to perform equally well while a marked reduction in the amount of ligated product was observed for the 1 mM condition. Importantly, the MgCl₂ released from the droplet library (lanes 1-3) appeared equally capable of catalyzing the ligation reaction as freshly added MgCl₂ (lanes 4-6). NOTE: The 50 mM droplet condition looks slightly less intense on the gel image due to slightly less material being loaded on the gel for that lane.

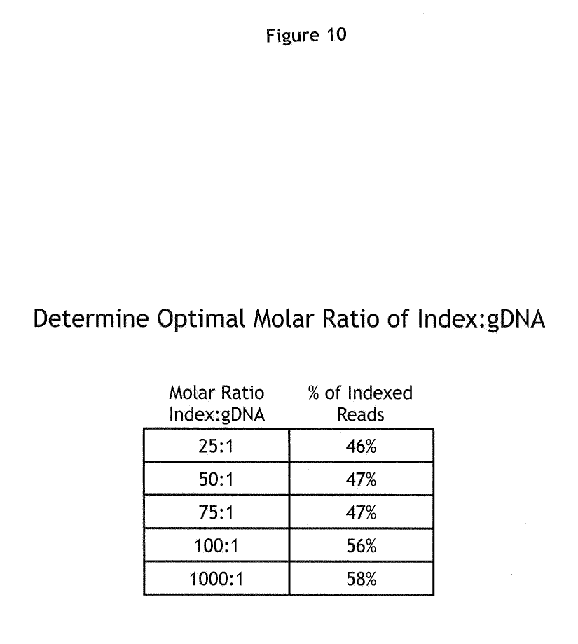

FIG. 10 represents a determination of the optimal ratio of genomic Index:genomic DNA. Lambda genomic DNA was sheared to a mean size of ˜300 bp using a Covaris S2 instrument. The genomic DNA was then end repaired and utilized in a ligation reaction containing variable molar ratios of Index:gDNA. Following index ligation, samples were end repaired, A-tailed and ligated to Illumina adapters. Samples were pooled and sequenced on an Illumina MiSeq. The percentage of reads where indexed was observed is shown. NOTE: The indexes used in this experiment were blunt ended 20 bp sequences.

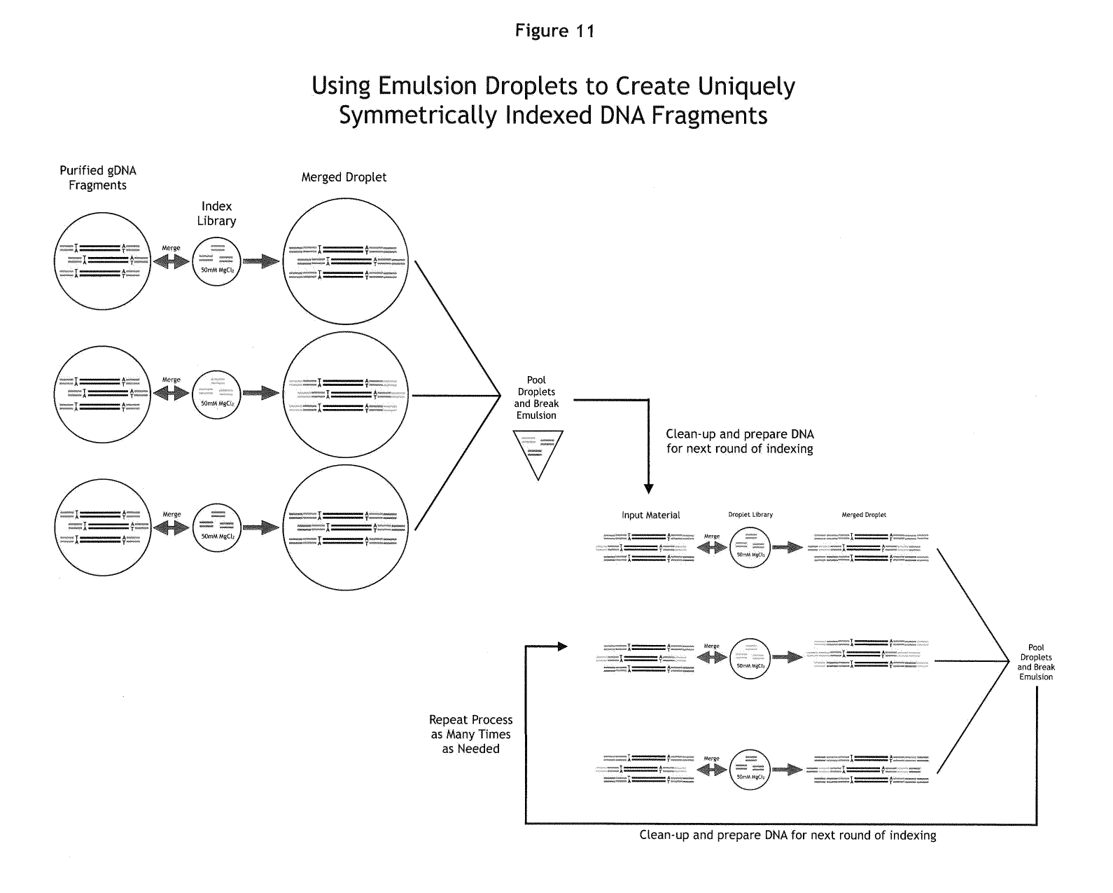

FIG. 11 provides the process of symmetric indexing in emulsion. Index libraries are prepared in an emulsion. The droplets carrying index also contain a concentration of MgCl₂ such that when they are joined with a solution of DNA, ligase buffer and ligase enzyme, the final concentration of MgCl₂ in a given reaction is 50 mM. Although many molecules/particles receive the same index in any given round of addition, the probability that any two identically indexed molecules/particles will travel together in a subsequent round of index addition is extremely low. After each round of index addition, the emulsion is broken and DNA samples are purified and prepared for the next round of index addition.

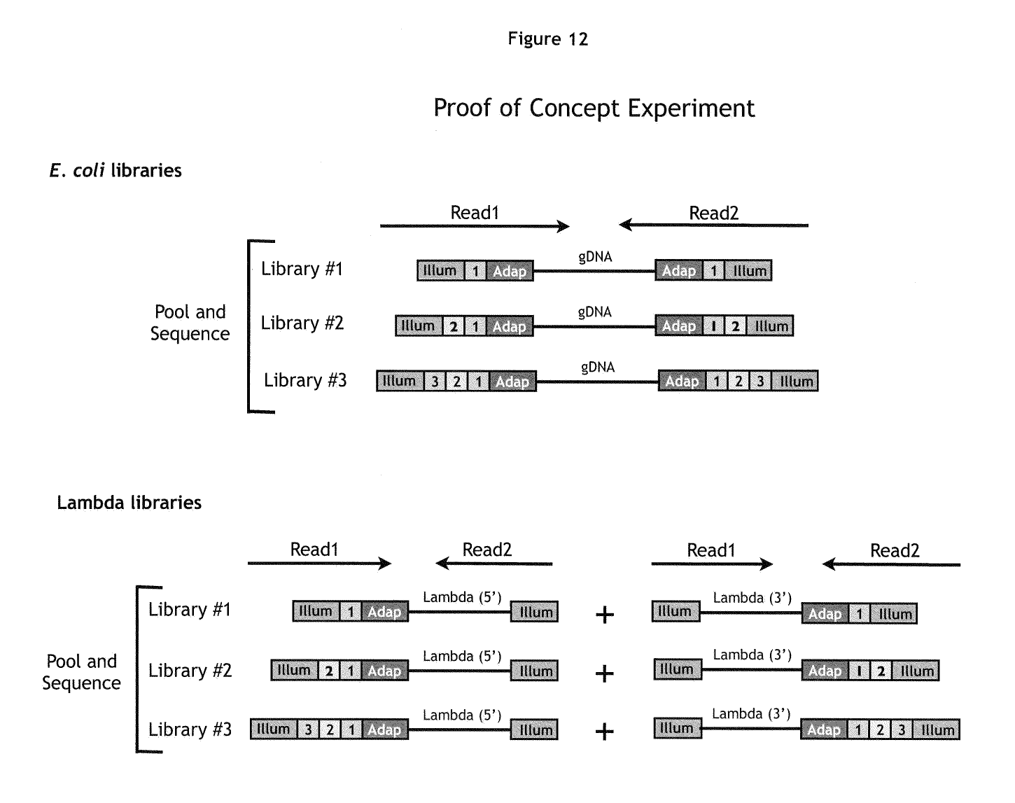

FIG. 12 shows a proof of concept experiment. E. coli genomic DNA was sheared to a mean size of approximately 300 bp using a Covaris S2 instrument, end repaired, A-tailed and ligated to the cap adapter. Lambda genomic DNA was prepared similarly, but was not sheared. Genomic DNA fragments were then subjected to 1, 2 or 3 rounds of blunt-ended index ligation in bulk (i.e., in microcentrifuge tubes). E. coli fragments were not sheared following index ligation while lambda fragments were sheared to approximately 500 bp using a Covaris S2 instrument. Cap containing fragments were selected via incubation with paramagnetic streptavidin M-280 beads, end repaired, A-tailed and ligated to Illumina sequencing adapters. Samples were pooled and sequenced on an Illumina MiSeq using standard paired end chemistry.

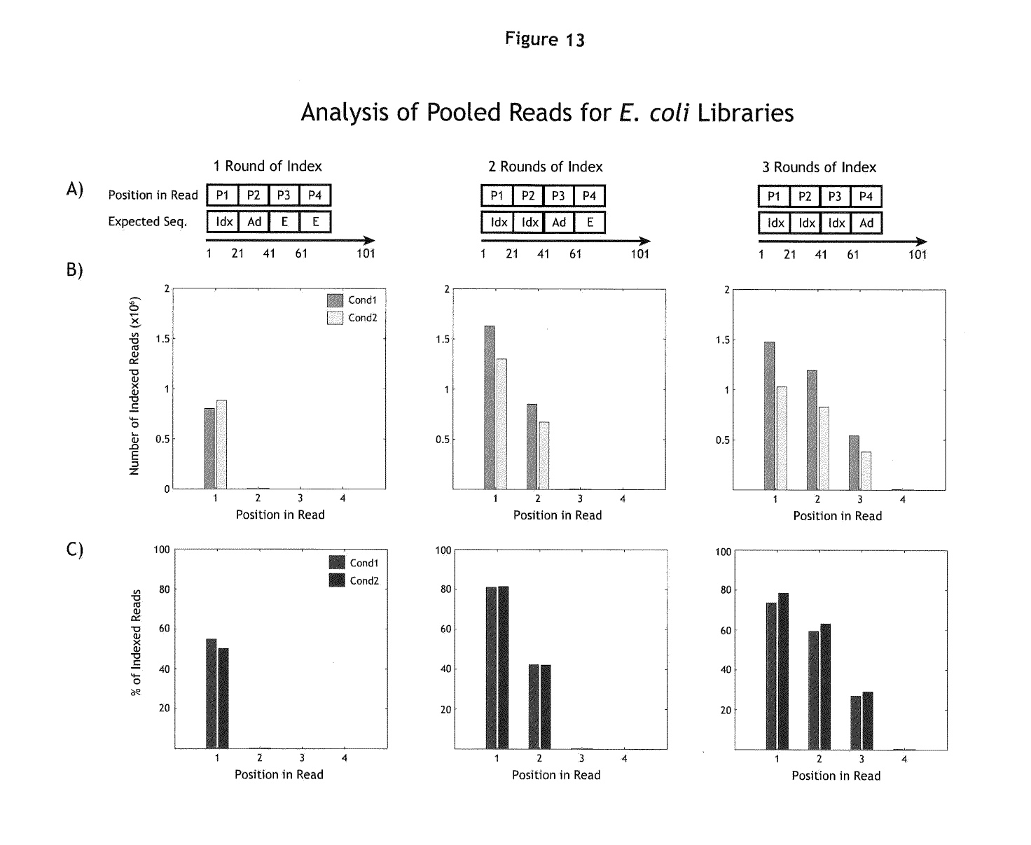

FIG. 13 is an analysis of E. coli proof of concept libraries. E. coli genomic DNA libraries were prepared in duplicate (Cond1 and Cond2) as described above (see FIG. 12). Libraries were pooled and sequenced with a 101 bp paired read on a single MiSeq run. Paired reads that passed filter were analyzed together as a single population. (A) Reads were broken down into 20 bp units (i.e., positions) and checked for the presence of index sequences. The expected outcome at a given position is shown (Idx=index, Ad=capping adapter, E=E. coli genomic DNA). (B) The number of reads containing index at a given position are shown. (C) The percent of reads containing index at a given position is shown.

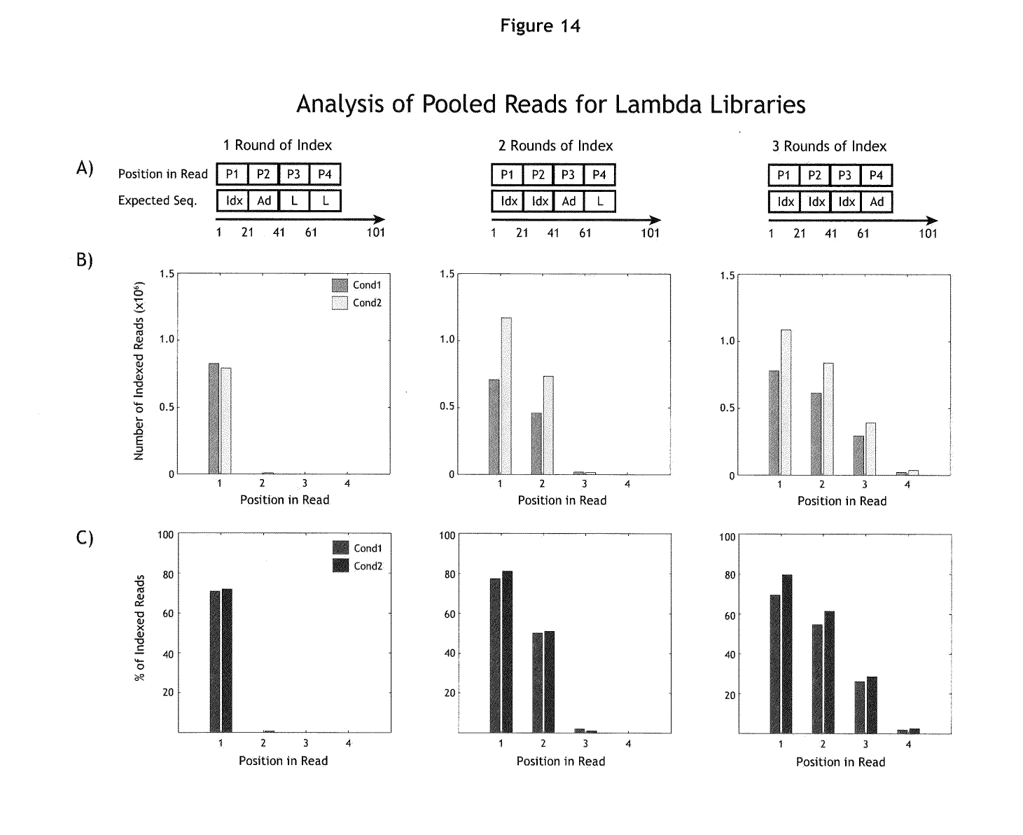

FIG. 14 provides an analysis of lambda proof of concept libraries. Lambda phage genomic DNA libraries were prepared in duplicate (Cond1 and Cond2) as described above (see FIG. 12). Libraries were pooled and sequenced with a 101 bp paired read on a single MiSeq run. Paired reads that passed filter were analyzed together as a single population. (A) Reads were broken down into 20 bp units (i.e., positions) and checked for the presence of index sequences. The expected outcome at a given position is shown (Idx=index, Ad=capping adapter, L=lambda genomic DNA). (B) The number of reads containing index at a given position are shown. (C) The percent of reads containing index at a given position is shown. The percentages shown are corrected for the fact that one half of the reads will necessarily be the genomic “end” of the library insert.

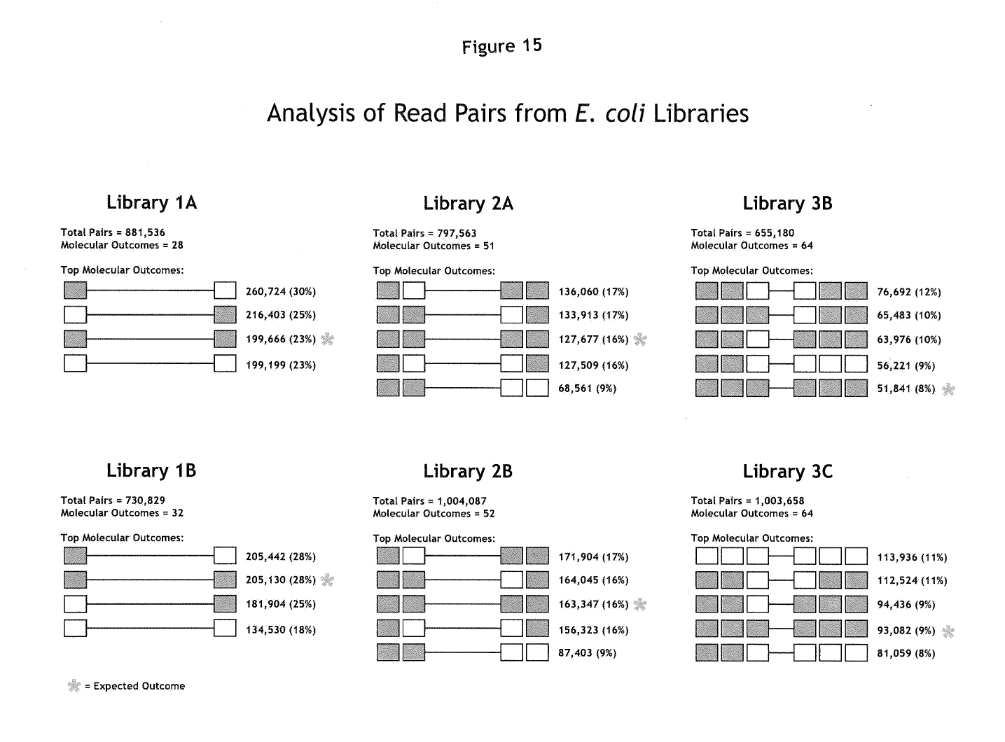

FIG. 15 shows a determination of symmetry of indexing in E. coli proof of concept libraries. E. coli genomic DNA was prepared as described above (see FIG. 12). Libraries subjected to 1 (panels 1A and 1B), 2 (panels 2A and 2B) or 3 (panels 3A and 3B) rounds of index ligation are shown. All data that passed filter was analyzed as read pairs which were then broken down into 20 bp units (i.e., positions) and checked for the presence of index sequences. Positions where index sequences were detected are depicted by green boxes; positions where indexes were not detected are depicted by white boxes. The expected outcome for each library is denoted by a green asterisk.

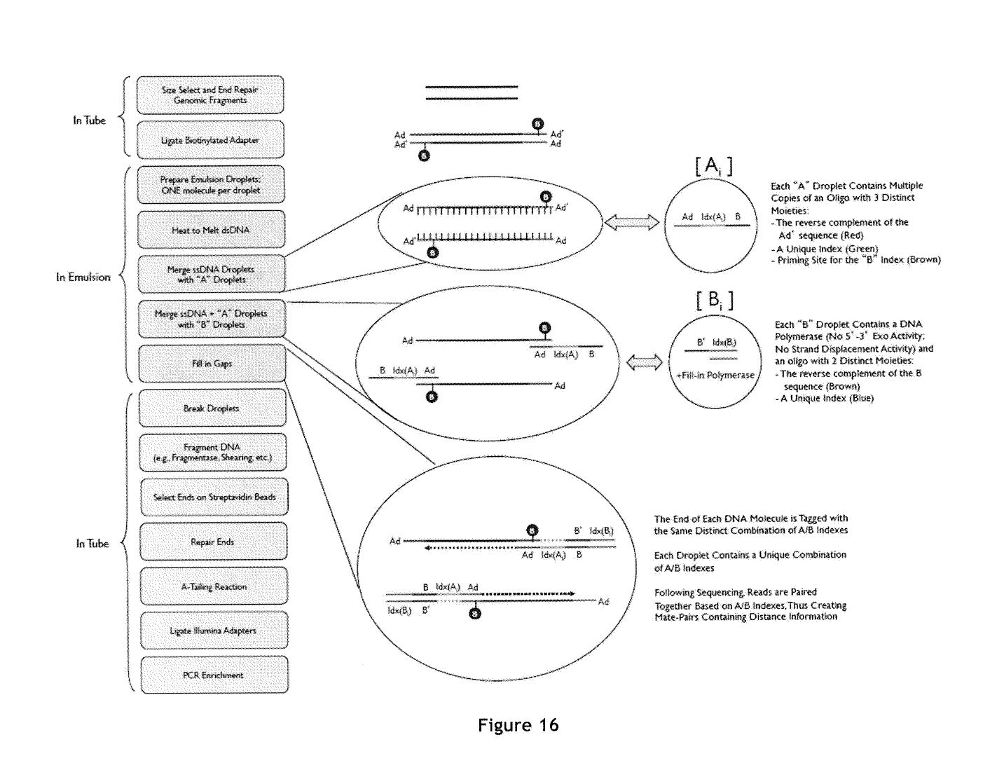

FIG. 16 is a schematic representation of a mate pair synthesis process using single stranded genomic DNA as the agent. Each droplet may comprise both strands of the genomic fragment. As shown in the Figure, the strands are identically labeled at one end.

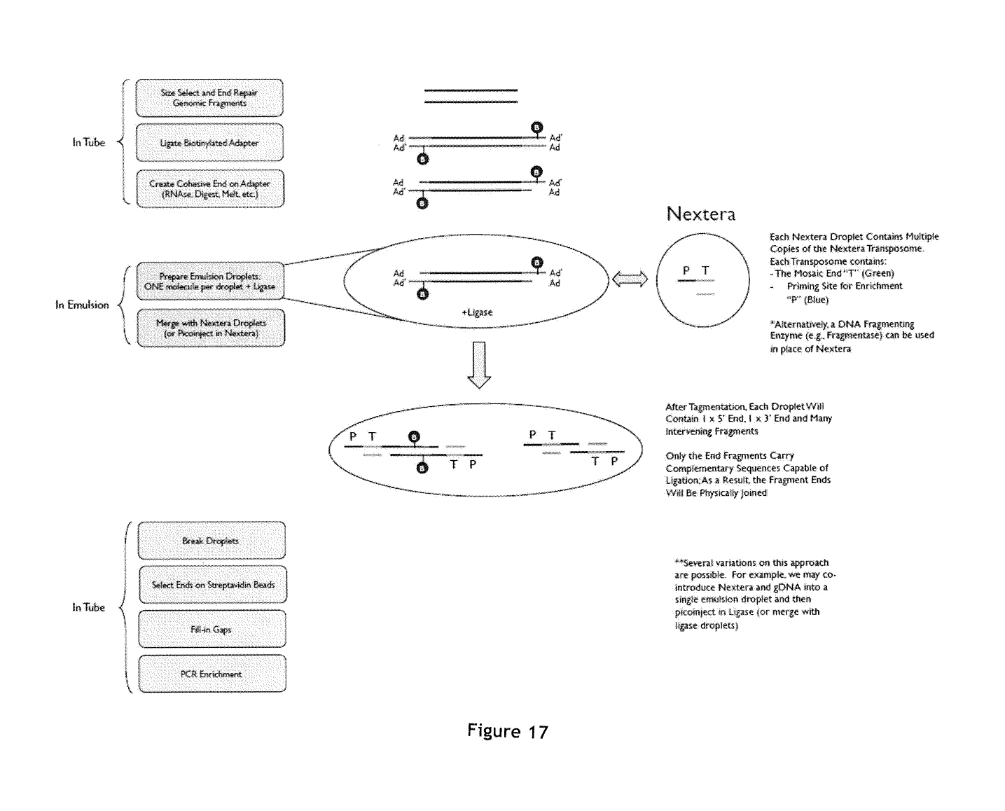

FIG. 17 is a schematic of a mate pair synthesis using droplets and Nextera transposomes as detectable tags.

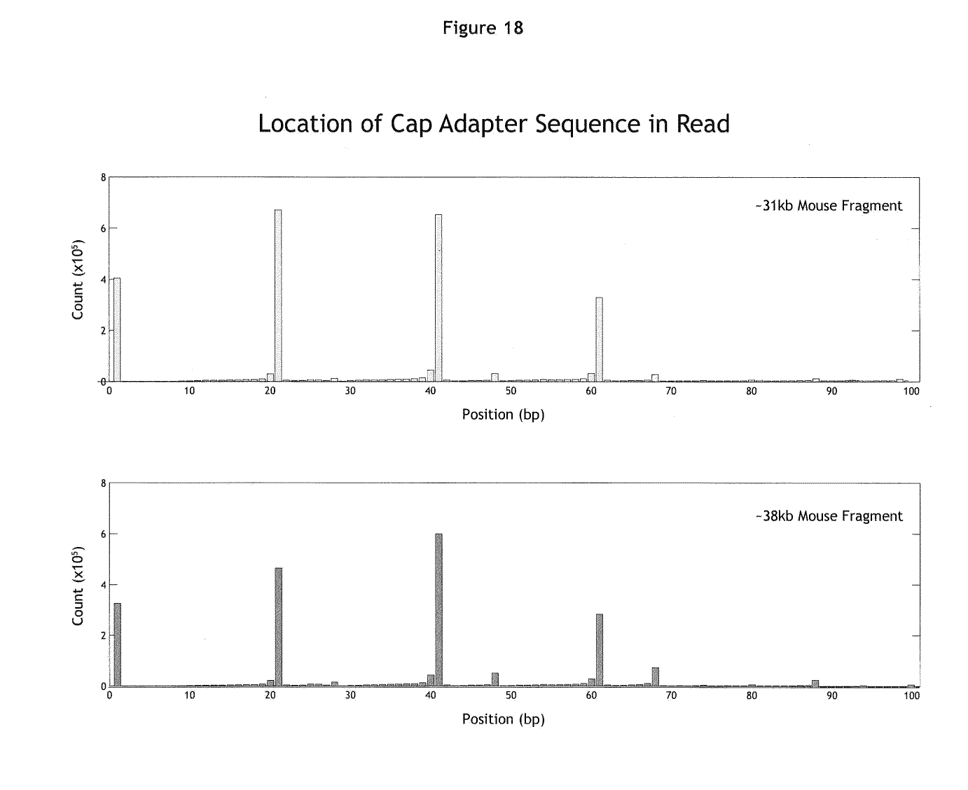

FIG. 18 shows the determination of uniformity of blunt-ended indexing. C57BL/6J mouse genomic DNA was sheared to approximately 40 kb using a Genemachines Hydroshear. Samples were run on a 0.7% agarose gel and fragments of approximately 31 kb and 38 kb were collected and purified separately. All fragments were then end repaired, A-tailed and ligated to the biotinylated cap adapter. Fragments were then ligated to blunt-ended index sequences contained in a droplet library using the Raindance Thunderstorm instrument. Following each round of index ligation, the emulsion was broken and samples were end repaired and purified for use in subsequent rounds of ligation. A total of 3 rounds of index ligation were performed. After the final round of index ligation, samples were sheared to ˜500 bp in length using a Covaris S2 instrument. Fragments containing the biotinylated cap adapter were selected using streptavidin M-280 beads, end repaired, A-tailed and ligated to Illumina sequencing adapters. Samples were then sequenced using an Illumina MiSeq instrument. The location of the cap sequence within a given read was determined. The total number of reads with cap sequence identified at a given position within the read are shown. Significant populations of reads where the cap was located at bp 1 (i.e., no index present), bp 21 (1 index), bp 41 (2 indexes) and bp 61 (3 indexes) were observed for both the 31 kb and 38 kb libraries.

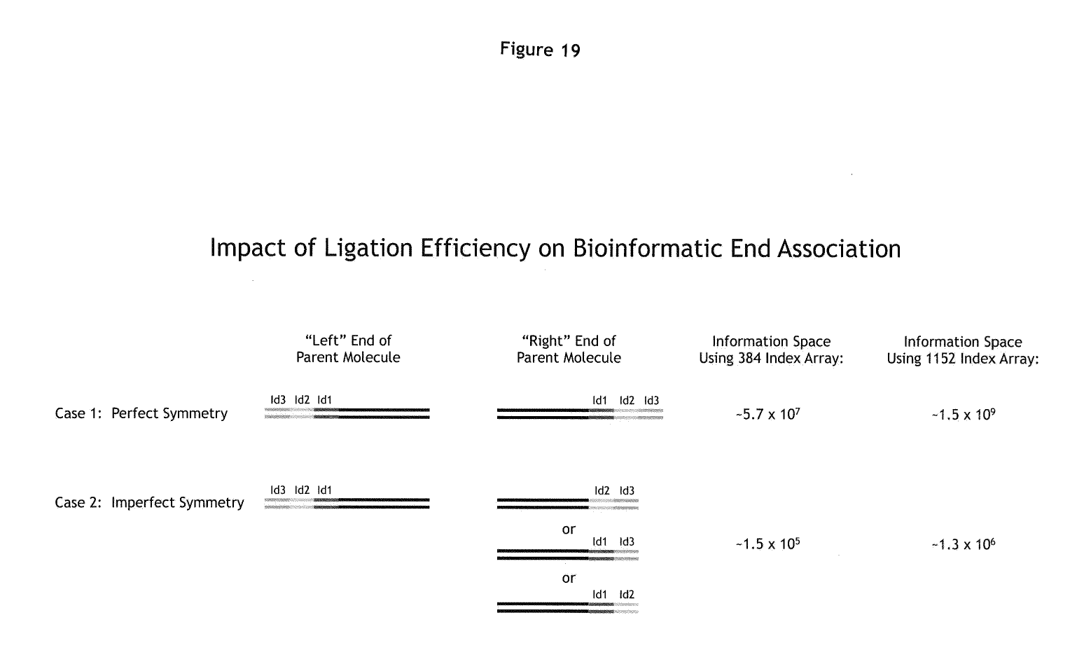

FIG. 19 describes impact of ligation efficiency on bioinformatics end association. Upon analysis of the indexed mouse libraries (see FIG. 18), it was observed that clear populations of reads carrying 1, 2 or 3 indexes were present in the final library. The fact that multiple distinct populations were observed indicates a lack of symmetry in the indexing protocol. This presents a challenge to the informatic association of mate pairs since, for example, a read carrying 3 indexes may have its mate in the pool of reads carrying 3, 2 or 1 indexes. Thus, the likelihood of correctly pairing reads decreases as the number of indexes present decreases.

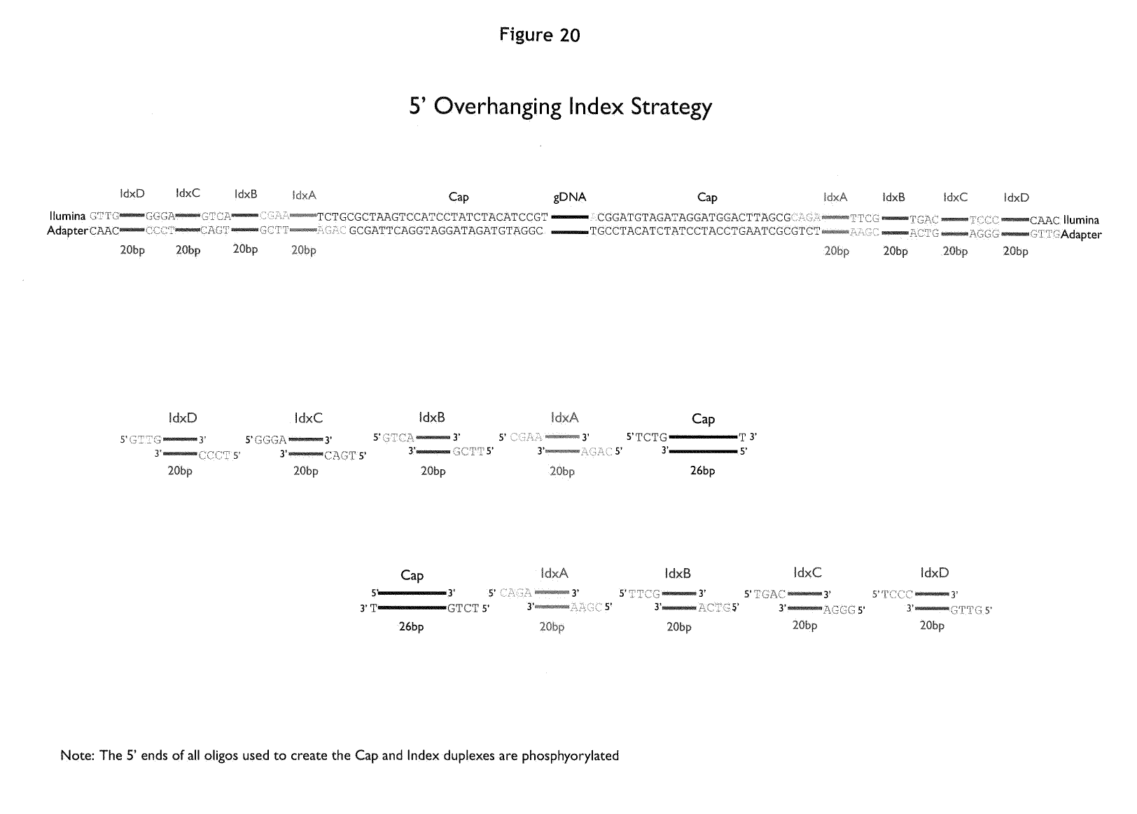

FIG. 20 is a redesign of index sequences to improve ligation efficiency. The cap adapter and all barcodes were redesigned to carry a 4-bp cohesive overhang on either side of the barcode (but only on one side of the cap adapter). Barcodes were separated into 4 different populations (A, B, C and D) depending on the sequence of the 4-bp cohesive overhang. The sequence of the cohesive overhang for each population is shown.

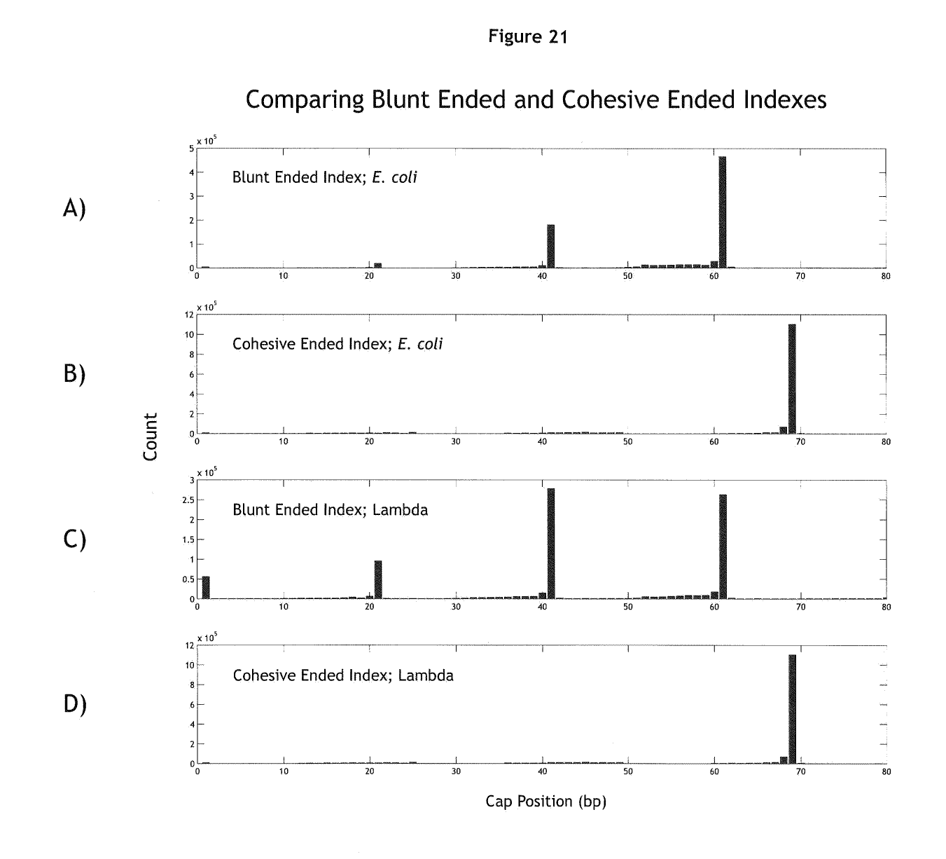

FIG. 21 shows uniformity of indexing using cohesive overhang indexing. E. coli and lambda genomic DNA libraries were prepared as described above (see FIG. 12), but this time using cohesive overhang indexes in conjunction with a cohesive overhang ended cap adapter. Reads were analyzed as before and the location of the biotinylated cap within the read was determined. The total number of reads with cap sequence identified at a given position within the read are shown. This analysis revealed a clear improvement in the homogeneity of the indexed read population where the vast majority of the reads from the cohesive overhang indexed libraries carried 3 indexes

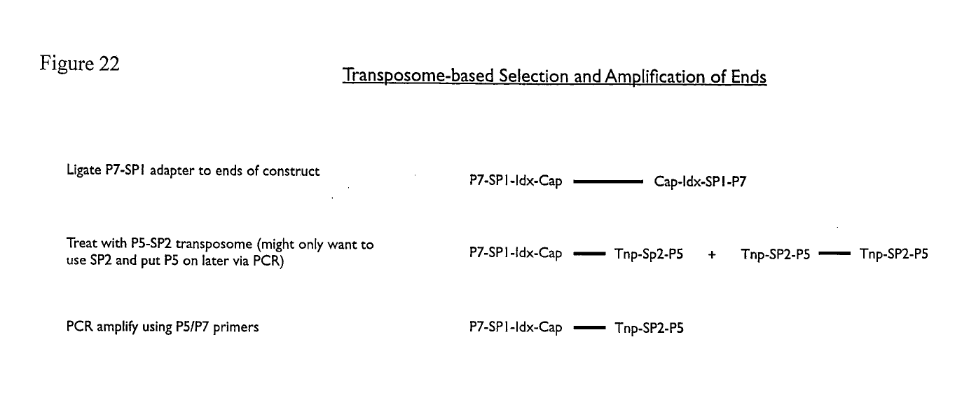

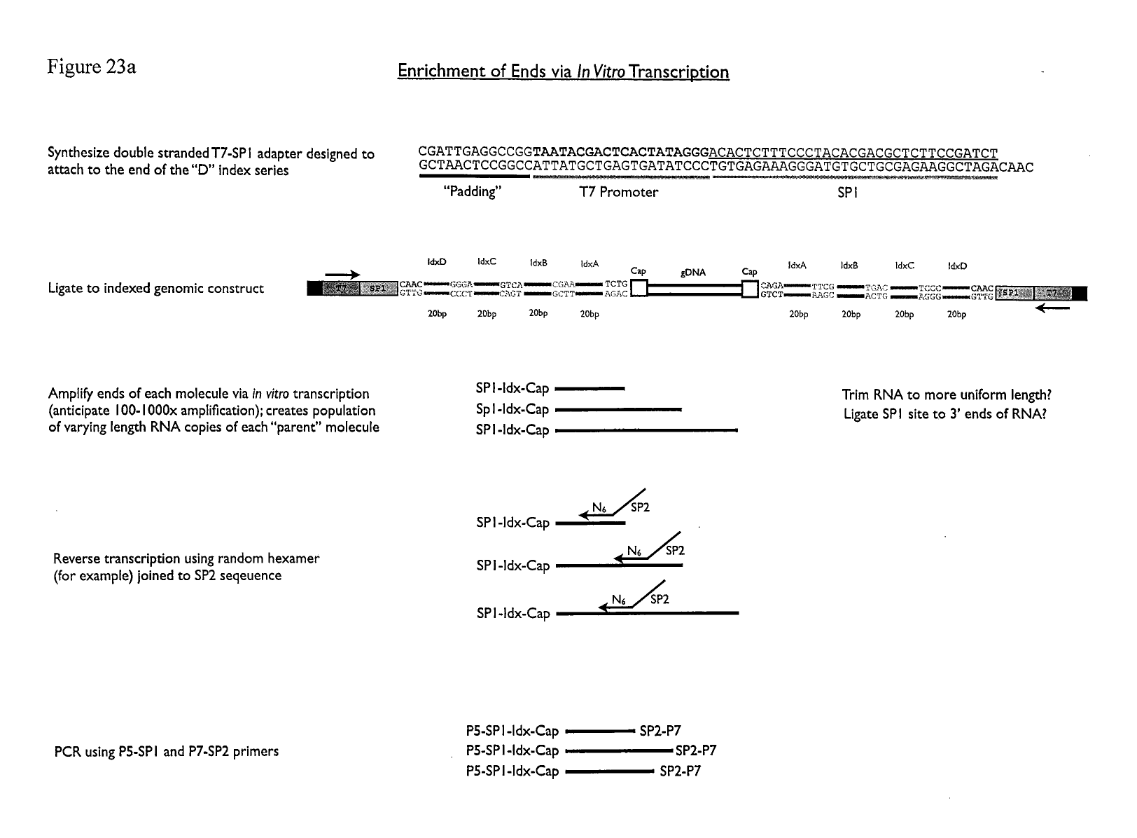

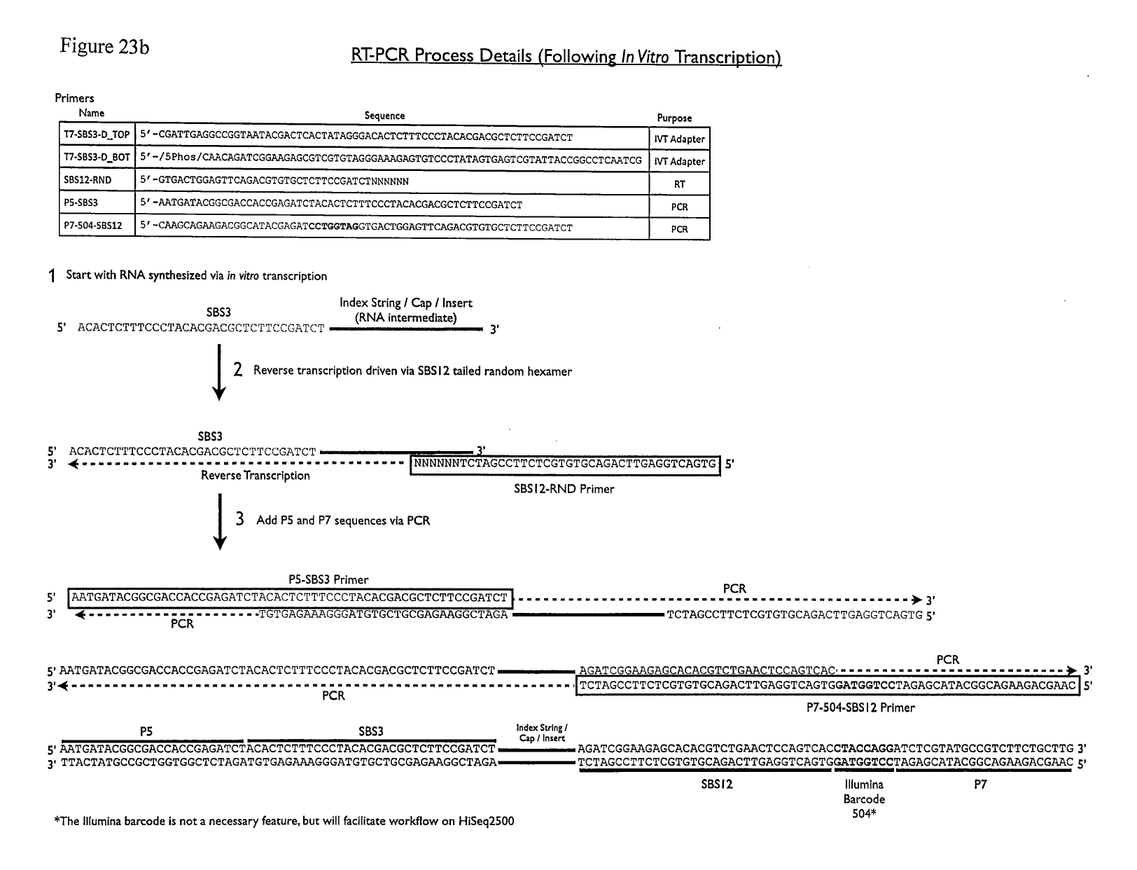

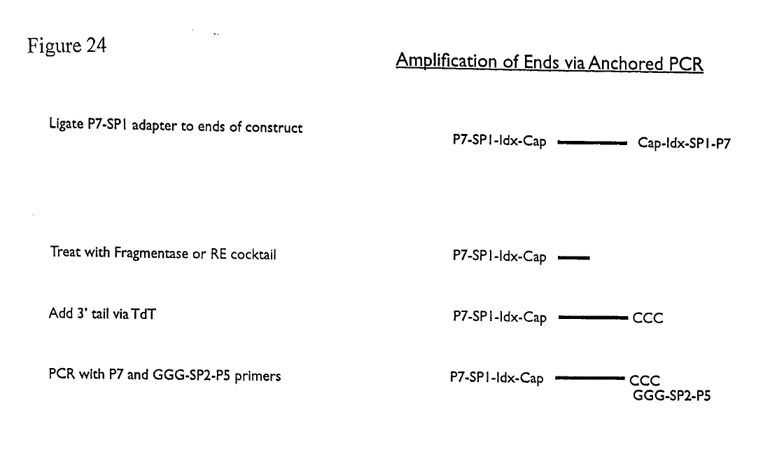

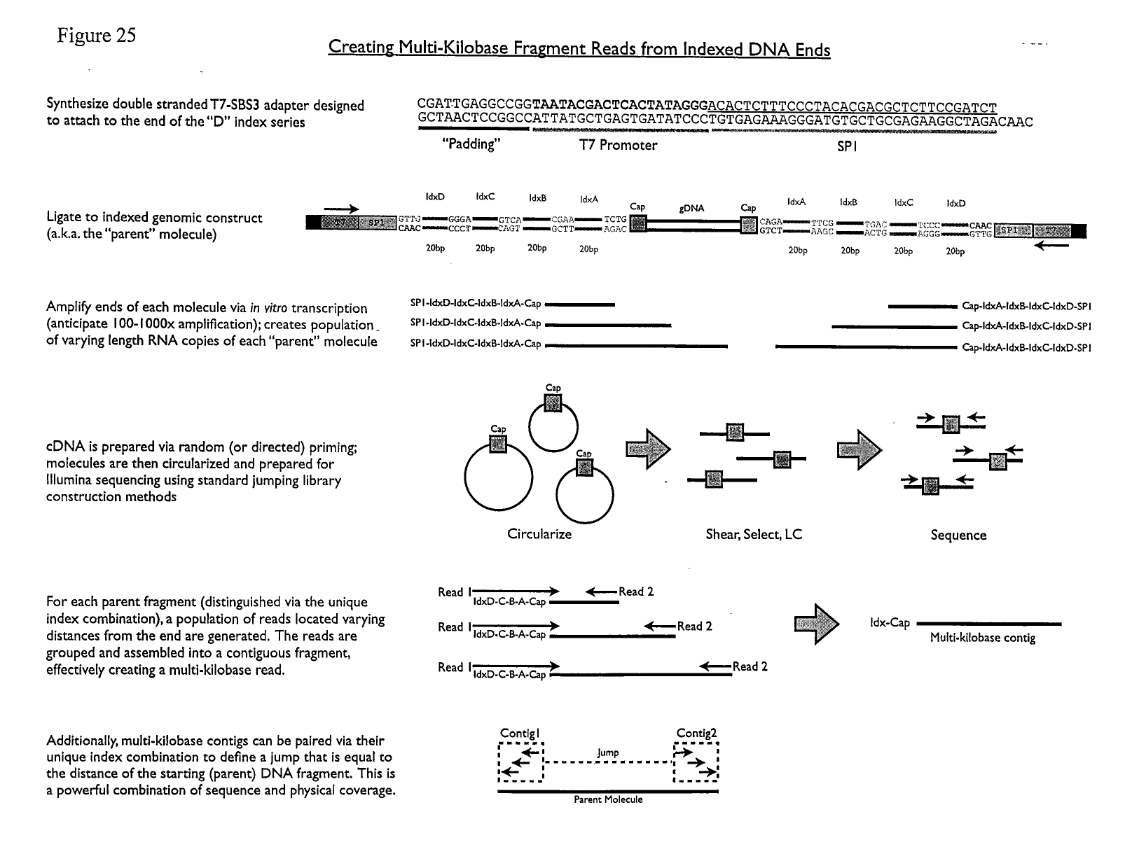

FIGS. 22 to 24 show examples of fragment amplification techniques. FIG. 22 shows transposome-based selection and amplification of ends creating many fragments where both ends are flanked by an Illumina P5 sequence. FIG. 23 shows enrichment of ends via in vitro transcription wherein T7 RNA polymerase is used in order to amplify both ends of a given molecule. FIG. 24 shows amplification using an anchored PCR technique described in Example 3.

Thus, in one aspect, the invention provides a method which may comprise generating a plurality of unique labels by attaching at least two, randomly selected, detectable oligonucleotide tags to each other in a sequential manner, and associating each unique label with a separate nucleic acid.

In some embodiments, the at least two detectable tags are attached to each other using ligation, polymerization, or a combination thereof.

In some embodiments, the unique label is generated in an emulsion droplet or in a series of emulsion droplets. In some embodiments, the library of uniquely-labeled nucleic acids is generated using an emulsion droplet or a series of emulsion droplets.

In another aspect, the invention provides a method which may comprise sequentially attaching at least two detectable oligonucleotide tags to a 5′ and/or 3′ end of a first nucleic acid, wherein each detectable oligonucleotide tag is randomly selected from a plurality of detectable oligonucleotide tags, thereby generating a second nucleic acid which may comprise the first nucleic acid attached at its 5′ and/or 3′ end with a unique combination of detectable oligonucleotide tags.

In some embodiments, the first nucleic acid is a genomic DNA fragment.

In another aspect method which may comprise sequentially end-labeling nucleic acids in a plurality, at their 5′ and 3′ ends, with a random combination of n detectable oligonucleotide tags, wherein each end-labeled nucleic acid is (a) identically labeled at its 5′ and 3′ ends, and (b) uniquely labeled relative to other nucleic acids in the plurality, wherein each detectable oligonucleotide tags is randomly and independently selected from a number of detectable oligonucleotide tags that is less than the number of nucleic acids, and n is the number of oligonucleotides attached to an end of a nucleic acid.

In some embodiments, the number of oligonucleotides is 10-fold, 100-fold, 1000-fold, or 10000-fold less than the number of nucleic acids.

In some embodiments, the method further may comprise fragmenting end-labeled nucleic acids into at least a 5′ fragment which may comprise the 5′ end of the nucleic acid attached to the random combination of n oligonucleotide tags and into a 3′ fragment which may comprise the 3′ end of the nucleic acid attached to the random combination of n oligonucleotide tags.

In some embodiments, the 5′ and 3′ fragments are about 10-1000 bases (base pairs) in length, or about 10-500 bases in length, or about 10-200 bases in length. In some embodiments, the method further may comprise sequencing the 5′ and 3′ fragments.

In another aspect, the invention provides a method which may comprise (a) end-labeling two or more first subsets of nucleic acids with a detectable oligonucleotide tag to produce nucleic acids within a subset that are identically end-labeled relative to each other and uniquely end-labeled relative to nucleic acids in other subsets; (b) combining two or more subsets of uniquely end-labeled nucleic acids to form a pool of nucleic acids, wherein the pool may comprise two or more second subsets of nucleic acids that are distinct from the two or more first subsets of nucleic acids; (c) identically end-labeling two or more second subsets of nucleic acids with a second detectable oligonucleotide tag to produce nucleic acids within a second subset that are uniquely labeled relative to nucleic acids in the same or different second subsets; and (d) repeating steps (b) and (c) until a number of unique end-label combinations is generated that exceeds the number of starting nucleic acids.

In another aspect, the invention provides a method which may comprise (a) providing a pool of nucleic acids; (b) separating the pool of nucleic acids into sub-pools of nucleic acids; (c) end-labeling nucleic acids in each sub-pool of with one of m₁ detectable oligonucleotide tags thereby producing sub-pools of labeled nucleic acids, wherein nucleic acids in a sub-pool are identically end-labeled to each other, (d) combining sub-pools of labeled nucleic acids to create a pool of labeled nucleic acids; (e) separating the pool of labeled nucleic acid molecules into second sub-pools of labeled nucleic acids; (f) repeating steps (c) to (e) n times to produce nucleic acids end-labeled with n detectable oligonucleotide tags wherein the pool in (a) consists of a number of nucleic acids that is less than (m₁)(m₂)(m₃) . . . (mₙ).

In another aspect, the invention provides a method which may comprise (a) providing a population of library droplets which may comprise nucleic acids, wherein each droplet may comprise a nucleic acid; (b) fusing each individual library droplet with a single index droplet from a plurality of m₁ index droplets, each index droplet which may comprise a plurality of one unique detectable oligonucleotide tag; (c) end-labeling the nucleic acid with the unique detectable oligonucleotide tag in a fused droplet; (d) harvesting end-labeled nucleic acids from the fused droplets and generating another population of library droplets which may comprise end-labeled nucleic acids; and (e) repeating steps (b) to (d) n times to produce nucleic acids end-labeled with n unique detectable oligonucleotide tag, wherein the n unique detectable oligonucleotide tags generate an (m₁)(m₂)(m₃) . . . (mₙ) number of combinations that is greater than the number of starting nucleic acids.

In some embodiments, end-labeling may comprise ligation of the unique oligonucleotide tag with the nucleic acid. In some embodiments, the unique oligonucleotide tag is double-stranded. In some embodiments, the method further may comprise phosphorylating the nucleic acids between steps (b) and (c).

In some embodiments, end-labeling may comprise a polymerase-mediated fill-in reaction. In some embodiments, the polymerase-mediated fill-in reaction may comprise (a) producing a single-stranded cohesive overhang on the nucleic acid, wherein the cohesive overhang is complementary to one end of the unique detectable oligonucleotide tag; (b) annealing the complementary end of the unique oligonucleotide tag to the single-stranded cohesive overhang such that at least one nucleotide of the unique detectable oligonucleotide tag is not annealed to the nucleic acid, producing a unique detectable oligonucleotide tag cohesive overhang; and (c) extending the single-stranded cohesive overhang of (a) using a polymerase and nucleotides complementary to the unique detectable oligonucleotide tag cohesive overhang to produce a double-stranded unique detectable oligonucleotide tag.

In some embodiments, the single-stranded cohesive overhang on the nucleic acid is produced by a USER enzyme. In some embodiments, the unique detectable oligonucleotide tag is single-stranded.

In some embodiments, an oligonucleotide adapter is added to the nucleic acids before labeling with the unique detectable oligonucleotide tags. In some embodiments, the adapter may comprise biotin. In some embodiments, the adapter may comprise a thymidine tail cohesive overhang.

In some embodiments, labeling occurs at the 5′ and 3′ ends of the nucleic acid. In some embodiments, labeling occurs at the 5′ or the 3′ end of the nucleic acid.

In some embodiments, the nucleic acids are genomic DNA, cDNA, PCR products, or fragments thereof.

In some embodiments, the method further may comprise fragmenting uniquely end-labeled nucleic acids. In some embodiments, the method further may comprise sequencing the uniquely end-labeled nucleic acids.

In some embodiments, the number of nucleic acids in the pool is at least two times greater than the number of unique oligonucleotide tags.

In another aspect, the invention provides a method which may comprise (a) providing a population of library droplets which may comprise nucleic acids, wherein each droplet may comprise a nucleic acids end-labeled on its 5′ and 3′ ends with oligonucleotide label, wherein the oligonucleotide label on the 5′ end (the 5′ oligonucleotide label) and the oligonucleotide on the 3′ end (the 3′ oligonucleotide label) may comprise a nucleotide cohesive overhang, and wherein the nucleotide cohesive overhang on the 5′ oligonucleotide label is complementary to the nucleotide cohesive overhang on the 3′ oligonucleotide label; (b) fusing each individual library droplet with a droplet which may comprise a DNA fragmenting enzyme, thereby producing a fused droplet; (c) fragmenting the nucleic acid with the 5′ and 3′ oligonucleotide labels in the fused droplet, thereby producing a fused droplet which may comprise a nucleic acid fragment which may comprise the 5′ oligonucleotide label and a nucleic acid fragment which may comprise the 3′ oligonucleotide label; and (d) ligating the 5′ oligonucleotide label and the 3′ oligonucleotide label nucleic acid, thereby ligating the nucleic acid fragment which may comprise the 5′ oligonucleotide label and the nucleic acid fragment which may comprise the 3′ oligonucleotide label, thereby producing a ligated nucleic acid.

In some embodiments, the 5′ oligonucleotide label and/or the 3′ oligonucleotide may comprise a biotin label.

In some embodiments, the method further may comprise (e) sequencing the ligated nucleic acid.

In some embodiments, the DNA fragmenting agent is Nextera.

In another aspect, the invention provides a method which may comprise (a) providing a population of library droplets which may comprise nucleic acids, wherein each droplet may comprise a nucleic acid which may comprise an oligonucleotide adapter; (b) melting the nucleic acid; (c) fusing each individual library droplet which may comprise a melted nucleic acid with a single index droplet from a plurality of m1 index droplets, each index droplet which may comprise a first unique single-stranded detectable oligonucleotide tag, wherein the first unique single-stranded detectable oligonucleotide tag may comprise a region complementary to the oligonucleotide adapter, (d) annealing the first unique single-stranded detectable oligonucleotide tag to the nucleic acid and performing a fill-in reaction, thereby producing an end-labeled nucleic acid; (e) harvesting end-labeled nucleic acids from the fused droplets and generating another population of library droplets, wherein each droplet may comprise an end-labeled nucleic acid; (f) melting the end-labeled nucleic acid; (g) fusing each individual library droplet which may comprise a melted end-labeled nucleic acid with a single index droplet from a plurality of m2 index droplets, each index droplet which may comprise a second unique single-stranded detectable oligonucleotide tag, wherein the second unique single-stranded detectable oligonucleotide tag may comprise a region complementary to the first unique single-stranded detectable oligonucleotide tag; (h) annealing the second unique single-stranded detectable oligonucleotide tag to the nucleic acid and performing a fill-in reaction, thereby producing an end-labeled nucleic acid; (i) harvesting end-labeled nucleic acid molecules from the fused droplets and generating another population of library droplets which may comprise end-labeled nucleic acids; and (j) repeating steps (f) to (i) n times to produce nucleic acids end-labeled with n detectable oligonucleotide tags, wherein the n detectable oligonucleotide tags generate an (m₁)(m₂)(m₃) . . . (mₙ) number of combinations that is greater than the number of starting nucleic acids.

In another aspect, the invention provides a method which may comprise sequencing a pair of genomic nucleic acid fragments, wherein the genomic nucleic acid fragments are attached to identical unique labels at one of their ends that indicates the genomic nucleic acid fragments were separated by a known distance in a genome prior to fragmentation.

In some embodiments, the pair of nucleic acid fragments were separated by greater than 10 kb in the genome prior to fragmentation.

In some embodiments, the pair of nucleic acid fragments were separated by greater than 40 kb in the genome prior to fragmentation.

In some embodiments, the method further may comprise generating the pair of genomic nucleic acid fragments by fragmenting nucleic acids which may comprise genomic sequence and identical non-genomic sequence at their 5′ and 3′ ends.

In another aspect, the invention provides a composition which may comprise a plurality of paired nucleic acid fragments attached to unique labels at one end, wherein paired nucleic acid fragments:

(a) share an identical unique label at one end that is unique in the plurality, and (b) were separated from each other in a genome by a known distance prior to fragmentation.

In some embodiments, paired nucleic acid fragments were separated by greater than 10 kb in the genome prior to fragmentation. In some embodiments, paired nucleic acid fragments were separated by greater than 40 kb in the genome prior to fragmentation.

In another aspect, the invention provides a composition which may comprise a plurality of paired genomic nucleic acid fragments produced any of the foregoing methods.

The present invention further encompasses methods of making and/or using one or more of the embodiments described herein.

Other advantages and novel features of the present invention will become apparent from the following detailed description of various non-limiting embodiments of the invention when considered in conjunction with the accompanying Figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

As used herein, “agent” or “agents” refers to a nucleic acid. The nucleic acid agent may be single-stranded (ss) or double-stranded (ds), or it may be partially single-stranded and partially double-stranded. Nucleic acid agents include but are not limited to DNA such as genomic DNA fragments, PCR and other amplification products, RNA, cDNA, and the like. Nucleic acid agents may be fragments of larger nucleic acids such as but not limited to genomic DNA fragments.

### Association of Labels and Agents

An agent of interest may be associated with a unique label. As used herein, “associated” refers to a relationship between the agent and the unique label such that the unique label may be used to identify the agent, identify the source or origin of the agent, identify one or more conditions to which the agent has been exposed, etc. A label that is associated with an agent may be, for example, physical attached to the agent, either directly or indirectly, or it may be in the same defined, typically physically separate, volume as the agent. A defined volume may be an emulsion droplet, a well (of for example a multiwell plate), a tube, a container, and the like. It is to be understood that the defined volume will typically contain only one agent and the label with which it is associated, although a volume containing multiple agents with multiple copies of the label is also contemplated depending on the application.

An agent may be associated with a single copy of a unique label or it may be associated with multiple copies of the same unique label including for example 2, 3, 4, 5, 6, 7, 8, 9, 10, 100, 1000, 10,000, 100,000 or more copies of the same unique label. In this context, the label is considered unique because it is different from labels associated with other, different agents.

Attachment of labels to agents may be direct or indirect. The attachment chemistry will depend on the nature of the agent and/or any derivatisation or functionalisation applied to the agent. For example, labels can be directly attached through covalent attachment. The label may include a moiety, which may be a non-nucleotide chemical modification, to facilitate attachment. By way of non-limiting example, the label may include methylated nucleotides, uracil bases, phosphorothioate groups, ribonucleotides, diol linkages, disulphide linkages, etc., to enable covalent attachment to an agent.

In another example, a label can be attached to an agent via a linker or in another indirect manner. Examples of linkers, include, but are not limited to, carbon-containing chains, polyethylene glycol (PEG), nucleic acids, monosaccharide units, and peptides. The linkers may be cleavable under certain conditions. Cleavable linkers are discussed in greater detail herein.

Methods for attaching nucleic acids to each other, as for example attaching nucleic acid labels to nucleic acid agents, are known in the art. Such methods include but are not limited to ligation and polymerase-mediated attachment methods (see, e.g., U.S. Pat. Nos. 7,863,058 and 7,754,429; Green and Sambrook. Molecular Cloning: A Laboratory Manual, Fourth Edition, 2012; Current Protocols in Molecular Biology, and Current Protocols in Nucleic Acid Chemistry, all of which are incorporated herein by reference).

### Detectable Oligonucleotide Tags

The unique labels of the invention are, at least in part, nucleic acid in nature, and are generated by sequentially attaching two or more detectable oligonucleotide tags to each other. As used herein, a detectable oligonucleotide tag is an oligonucleotide that can be detected by sequencing of its nucleotide sequence and/or by detecting non-nucleic acid detectable moieties it may be attached to.

The oligonucleotides tags are typically randomly selected from a diverse plurality of oligonucleotide tags. In some instances, an oligonucleotide tag may be present once in a plurality or it may be present multiple times in a plurality. In the latter instance, the plurality of tags may be comprised of a number of subsets each which may comprise a plurality of identical tags. In some important embodiments, these subsets are physically separate from each other. Physical separation may be achieved by providing the subsets in separate droplets from an emulsion. It is the random selection and thus combination of oligonucleotide tags that results in a unique label. Accordingly, the number of distinct (i.e., different) oligonucleotide tags required to uniquely label a plurality of agents can be far less than the number of agents being labeled. This is particularly advantageous when the number of agents is large (e.g., when the agents are members of a library).

As used herein, the term “oligonucleotide” refers to a nucleic acid such as deoxyribonucleic acid (DNA), ribonucleic acid (RNA), or DNA/RNA hybrids and includes analogs of either DNA or RNA made from nucleotide analogs known in the art (see, e.g. U.S. Patent or Patent Application Publications: U.S. Pat. No. 7,399,845, U.S. Pat. No. 7,741,457, U.S. Pat. No. 8,022,193, U.S. Pat. No. 7,569,686, U.S. Pat. No. 7,335,765, U.S. Pat. No. 7,314,923, U.S. Pat. No. 7,335,765, and U.S. Pat. No. 7,816,333. US 20110009471, the entire contents of each of which are incorporated herein by reference). Oligonucleotides may be single-stranded (such as sense or antisense oligonucleotides), double-stranded, or partially single-stranded and partially double-stranded.

A unique nucleotide sequence may be a nucleotide sequence that is different (and thus distinguishable) from the sequence of each detectable oligonucleotide tag in a plurality of detectable oligonucleotide tags. A unique nucleotide sequence may also be a nucleotide sequence that is different (and thus distinguishable) from the sequence of each detectable oligonucleotide tag in a first plurality of detectable oligonucleotide tags but identical to the sequence of at least one detectable oligonucleotide tag in a second plurality of detectable oligonucleotide tags. A unique sequence may differ from other sequences by multiple bases (or base pairs). The multiple bases may be contiguous or non-contiguous. Methods for obtaining nucleotide sequences (e.g., sequencing methods) are described herein and/or are known in the art.

In some embodiments, detectable oligonucleotide tags comprise one or more of a ligation sequence, a priming sequence, a capture sequence, and a unique sequence (optionally referred to herein as an index sequence). A ligation sequence is a sequence complementary to a second nucleotide sequence which allows for ligation of the detectable oligonucleotide tag to another entity which may comprise the second nucleotide sequence, e.g., another detectable oligonucleotide tag or an oligonucleotide adapter. A priming sequence is a sequence complementary to a primer, e.g., an oligonucleotide primer used for an amplification reaction such as but not limited to PCR. A capture sequence is a sequence capable of being bound by a capture entity. A capture entity may be an oligonucleotide which may comprise a nucleotide sequence complementary to a capture sequence, e.g. a second detectable oligonucleotide tag. A capture entity may also be any other entity capable of binding to the capture sequence, e.g. an antibody or peptide. An index sequence is a sequence which may comprise a unique nucleotide sequence and/or a detectable moiety as described above.

“Complementary” is a term which is used to indicate a sufficient degree of complementarity between two nucleotide sequences such that stable and specific binding occurs between one and preferably more bases (or nucleotides, as the terms are used interchangeably herein) of the two sequences. For example, if a nucleotide in a first nucleotide sequence is capable of hydrogen bonding with a nucleotide in second nucleotide sequence, then the bases are considered to be complementary to each other. Complete (i.e., 100%) complementarity between a first nucleotide sequence and a second nucleotide is preferable, but not required for ligation, priming, or capture sequences.

Table 1 below provides examples of certain oligonucleotide tags of the invention:

TABLE 1

| Tag | Full Oligo Sequence (Top) | Full Oliqo Sequence (Bot) |
|---|---|---|
| GGATATCTGTGGATATCTGT | /5Phos/CAGAGGATATCTGTGGATATCTGT | /5Phos/CGAAACAGATATCCACAGATATCC |
| GGCTGCCATAGGCTGCCATA | /5Phos/CAGAGGCTGCCATAGGCTGCCATA | /5Phos/CGAATATGGCAGCCTATGGCAGCC |
| GGTATGTCAAGGTATGTCAA | /5Phos/CAGAGGTATGTCAAGGTATGTCAA | /5Phos/CGAATTGACATACCTTGACATACC |
| GGTGAAGGACGGTGAAGGAC | /5Phos/CAGAGGTGAAGGACGGTGAAGGAC | /5Phos/CGAAGTCCTTCACCGTCCTTCACC |
| GTACCAGCATGTACCAGCAT | /5Phos/CAGAGTACCAGCATGTACCAGCAT | /5Phos/CGAAATGCTGGTACATGCTGGTAC |
| GTATACATCCGTATACATCC | /5Phos/CAGAGTATACATCCGTATACATCC | /5Phos/CGAAGGATGTATACGGATGTATAC |
| GTATCGCTCAGTATCGCTCA | /5Phos/CAGAGTATCGCTCAGTATCGCTCA | /5Phos/CGAATGAGCGATACTGAGCGATAC |
| GTCCGATGGAGTCCGATGGA | /5Phos/CAGAGTCCGATGGAGTCCGATGGA | /5Phos/CGAATCCATCGGACTCCATCGGAC |
| GTGTGGAATTGTGTGGAATT | /5Phos/CAGAGTGTGGAATTGTGTGGAATT | /5Phos/CGAAAATTCCACACAATTCCACAC |
| GTGTGGCAGAGTGTGGCAGA | /5Phos/CAGAGTGTGGCAGAGTGTGGCAGA | /5Phos/CGAATCTGCCACACTCTGCCACAC |
| GTTACGGTGAGTTACGGTGA | /5Phos/CAGAGTTACGGTGAGTTACGGTGA | /5Phos/CGAATCACCGTAACTCACCGTAAC |
| GTTCGTACATGTTCGTACAT | /5Phos/CAGAGTTCGTACATGTTCGTACAT | /5Phos/CGAAATGTACGAACATGTACGAAC |
| TAACCGGTAATAACCGGTAA | /5Phos/CAGATAACCGGTAATAACCGGTAA | /5Phos/CGAATTACCGGTTATTACCGGTTA |
| TACAGAGTCATACAGAGTCA | /5Phos/CAGATACAGAGTCATACAGAGTCA | /5Phos/CGAATGACTCTGTATGACTCTGTA |
| TACCGAGATATACCGAGATA | /5Phos/CAGATACCGAGATATACCGAGATA | /5Phos/CGAATATCTCGGTATATCTCGGTA |
| TACGCATCGCTACGCATCGC | /5Phos/CAGATACGCATCGCTACGCATCGC | /5Phos/CGAAGCGATGCGTAGCGATGCGTA |
| TACGCGCTTGTACGCGCTTG | /5Phos/CAGATACGCGCTTGTACGCGCTTG | /5Phos/CGAACAAGCGCGTACAAGCGCGTA |
| TAGACTTCACTAGACTTCAC | /5Phos/CAGATAGACTTCACTAGACTTCAC | /5Phos/CGAAGTGAAGTCTAGTGAAGTCTA |
| TATCGCTTAGTATCGCTTAG | /5Phos/CAGATATCGCTTAGTATCGCTTAG | /5Phos/CGAACTAAGCGATACTAAGCGATA |
| TCACGATCCGTCACGATCCG | /5Phos/CAGATCACGATCCGTCACGATCCG | /5Phos/CGAACGGATCGTGACGGATCGTGA |
| TCAGCTTCGCTCAGCTTCGC | /5Phos/CAGATCAGCTTCGCTCAGCTTCGC | /5Phos/CGAAGCGAAGCTGAGCGAAGCTGA |
| TCCGGACTTCTCCGGACTTC | /5Phos/CAGATCCGGACTTCTCCGGACTTC | /5Phos/CGAAGAAGTCCGGAGAAGTCCGGA |
| TCCGGTATGGTCCGGTATGG | /5Phos/CAGATCCGGTATGGTCCGGTATGG | /5Phos/CGAACCATACCGGACCATACCGGA |
| TCCGTGGTGATCCGTGGTGA | /5Phos/CAGATCCGTGGTGATCCGTGGTGA | /5Phos/CGAATCACCACGGATCACCACGGA |
| TCCTCATCCATCCTCATCCA | /5Phos/CAGATCCTCATCCATCCTCATCCA | /5Phos/CGAATGGATGAGGATGGATGAGGA |
| TCCTTCTGGATCCTTCTGGA | /5Phos/CAGATCCTTCTGGATCCTTCTGGA | /5Phos/CGAATCCAGAAGGATCCAGAAGGA |
| TCGCTGTTGCTCGCTGTTGC | /5Phos/CAGATCGCTGTTGCTCGCTGTTGC | /5Phos/CGAAGCAACAGCGAGCAACAGCGA |
| TCGGAGCTTGTCGGAGCTTG | /5Phos/CAGATCGGAGCTTGTCGGAGCTTG | /5Phos/CGAACAAGCTCCGACAAGCTCCGA |
| TCGGCATGAGTCGGCATGAG | /5Phos/CAGATCGGCATGAGTCGGCATGAG | /5Phos/CGAACTCATGCCGACTCATGCCGA |
| TCGGTGGAACTCGGTGGAAC | /5Phos/CAGATCGGTGGAACTCGGTGGAAC | /5Phos/CGAAGTTCCACCGAGTTCCACCGA |
| TCGGTTGTAATCGGTTGTAA | /5Phos/CAGATCGGTTGTAATCGGTTGTAA | /5Phos/CGAATTACAACCGATTACAACCGA |
| TCGTGTGGCATCGTGTGGCA | /5Phos/CAGATCGTGTGGCATCGTGTGGCA | /5Phos/CGAATGCCACACGATGCCACACGA |
| TCTGTTCCGCTCTGTTCCGC | /5Phos/CAGATCTGTTCCGCTCTGTTCCGC | /5Phos/CGAAGCGGAACAGAGCGGAACAGA |
| TCTTGGCGTCTCTTGGCGTC | /5Phos/CAGATCTTGGCGTCTCTTGGCGTC | /5Phos/CGAAGACGCCAAGAGACGCCAAGA |
| TGATTGAGTCTGATTGAGTC | /5Phos/CAGATGATTGAGTCTGATTGAGTC | /5Phos/CGAAGACTCAATCAGACTCAATCA |
| TGCGTTGGCATGCGTTGGCA | /5Phos/CAGATGCGTTGGCATGCGTTGGCA | /5Phos/CGAATGCCAACGCATGCCAACGCA |
| TGGACATGCGTGGACATGCG | /5Phos/CAGATGGACATGCGTGGACATGCG | /5Phos/CGAACGCATGTCCACGCATGTCCA |
| TGGCAAGCCATGGCAAGCCA | /5Phos/CAGATGGCAAGCCATGGCAAGCCA | /5Phos/CGAATGGCTTGCCATGGCTTGCCA |
| TGGTTAACTGTGGTTAACTG | /5Phos/CAGATGGTTAACTGTGGTTAACTG | /5Phos/CGAACAGTTAACCACAGTTAACCA |
| TGTACCTGAGTGTACCTGAG | /5Phos/CAGATGTACCTGAGTGTACCTGAG | /5Phos/CGAACTCAGGTACACTCAGGTACA |
| TGTACCTGAGTGTACCTGAG | /5Phos/CAGATGTACCTGAGTGTACCTGAG | /5Phos/CGAACTCAGGTACACTCAGGTACA |
| TGTACTACAGTGTACTACAG | /5Phos/CAGATGTACTACAGTGTACTACAG | /5Phos/CGAACTGTAGTACACTGTAGTACA |
| TGTCGGTTGCTGTCGGTTGC | /5Phos/CAGATGTCGGTTGCTGTCGGTTGC | /5Phos/CGAAGCAACCGACAGCAACCGACA |
| TGTCTGTCGGTGTCTGTCGG | /5Phos/CAGATGTCTGTCGGTGTCTGTCGG | /5Phos/CGAACCGACAGACACCGACAGACA |
| TGTGACTATCTGTGACTATC | /5Phos/CAGATGTGACTATCTGTGACTATC | /5Phos/CGAAGATAGTCACAGATAGTCACA |
| TGTGAGTGCGTGTGAGTGCG | /5Phos/CAGATGTGAGTGCGTGTGAGTGCG | /5Phos/CGAACGCACTCACACGCACTCACA |
| TGTGGTTCGCTGTGGTTCGC | /5Phos/CAGATGTGGTTCGCTGTGGTTCGC | /5Phos/CGAAGCGAACCACAGCGAACCACA |
| TGTTCTCTACTGTTCTCTAC | /5Phos/CAGATGTTCTCTACTGTTCTCTAC | /5Phos/CGAAGTAGAGAACAGTAGAGAACA |
| TTAGCCAGTCTTAGCCAGTC | /5Phos/CAGATTAGCCAGTCTTAGCCAGTC | /5Phos/CGAAGACTGGCTAAGACTGGCTAA |
| TTCGCGAATATTCGCGAATA | /5Phos/CAGATTCGCGAATATTCGCGAATA | /5Phos/CGAATATTCGCGAATATTCGCGAA |
| TTCGCGGTACTTCGCGGTAC | /5Phos/CAGATTCGCGGTACTTCGCGGTAC | /5Phos/CGAAGTACCGCGAAGTACCGCGAA |
| TTCTATGCAGTTCTATGCAG | /5Phos/CAGATTCTATGCAGTTCTATGCAG | /5Phos/CGAACTGCATAGAACTGCATAGAA |
| TTCTTACCGATTCTTACCGA | /5Phos/CAGATTCTTACCGATTCTTACCGA | /5Phos/CGAATCGGTAAGAATCGGTAAGAA |
| TTGCTCTGGATTGCTCTGGA | /5Phos/CAGATTGCTCTGGATTGCTCTGGA | /5Phos/CGAATCCAGAGCAATCCAGAGCAA |
| TTGTAACACCTTGTAACACC | /5Phos/CAGATTGTAACACCTTGTAACACC | /5Phos/CGAAGGTGTTACAAGGTGTTACAA |
| AACAAGTTCCAACAAGTTCC | /5Phos/CAGAAACAAGTTCCAACAAGTTCC | /5Phos/CGAAGGAACTTGTTGGAACTTGTT |
| AACAGCACGCAACAGCACGC | /5Phos/CAGAAACAGCACGCAACAGCACGC | /5Phos/CGAAGCGTGCTGTTGCGTGCTGTT |
| AACGTGCGGTAACGTGCGGT | /5Phos/CAGAAACGTGCGGTAACGTGCGGT | /5Phos/CGAAACCGCACGTTACCGCACGTT |
| AAGAGCGATGAAGAGCGATG | /5Phos/CAGAAAGAGCGATGAAGAGCGATG | /5Phos/CGAACATCGCTCTTCATCGCTCTT |
| AAGGAATTCCAAGGAATTCC | /5Phos/CAGAAAGGAATTCCAAGGAATTCC | /5Phos/CGAAGGAATTCCTTGGAATTCCTT |
| AAGTGAGGCGAAGTGAGGCG | /5Phos/CAGAAAGTGAGGCGAAGTGAGGCG | /5Phos/CGAACGCCTCACTTCGCCTCACTT |
| AATACGCGATAATACGCGAT | /5Phos/CAGAAATACGCGATAATACGCGAT | /5Phos/CGAAATCGCGTATTATCGCGTATT |
| AATAGGTTGGAATAGGTTGG | /5Phos/CAGAAATAGGTTGGAATAGGTTGG | /5Phos/CGAACCAACCTATTCCAACCTATT |
| AATCGAAGCTAATCGAAGCT | /5Phos/CAGAAATCGAAGCTAATCGAAGCT | /5Phos/CGAAAGCTTCGATTAGCTTCGATT |
| ACACTACGATACACTACGAT | /5Phos/CAGAACACTACGATACACTACGAT | /5Phos/CGAAATCGTAGTGTATCGTAGTGT |
| ACACTCAGCCACACTCAGCC | /5Phos/CAGAACACTCAGCCACACTCAGCC | /5Phos/CGAAGGCTGAGTGTGGCTGAGTGT |
| ACATCGAGCCACATCGAGCC | /5Phos/CAGAACATCGAGCCACATCGAGCC | /5Phos/CGAAGGCTCGATGTGGCTCGATGT |
| ACATTATGCGACATTATGCG | /5Phos/CAGAACATTATGCGACATTATGCG | /5Phos/CGAACGCATAATGTCGCATAATGT |
| ACCAGCAACTACCAGCAACT | /5Phos/CAGAACCAGCAACTACCAGCAACT | /5Phos/CGAAAGTTGCTGGTAGTTGCTGGT |
| ACCGAACACGACCGAACACG | /5Phos/CAGAACCGAACACGACCGAACACG | /5Phos/CGAACGTGTTCGGTCGTGTTCGGT |
| ACCGAACGGTACCGAACGGT | /5Phos/CAGAACCGAACGGTACCGAACGGT | /5Phos/CGAAACCGTTCGGTACCGTTCGGT |
| ACGAGTGGCTACGAGTGGCT | /5Phos/CAGAACGAGTGGCTACGAGTGGCT | /5Phos/CGAAAGCCACTCGTAGCCACTCGT |
| ACGGTTACGGACGGTTACGG | /5Phos/CAGAACGGTTACGGACGGTTACGG | /5Phos/CGAACCGTAACCGTCCGTAACCGT |
| ACGTAAGCGCACGTAAGCGC | /5Phos/CAGAACGTAAGCGCACGTAAGCGC | /5Phos/CGAAGCGCTTACGTGCGCTTACGT |
| ACTATGTGCTACTATGTGCT | /5Phos/CAGAACTATGTGCTACTATGTGCT | /5Phos/CGAAAGCACATAGTAGCACATAGT |
| ACTGGAGTCCACTGGAGTCC | /5Phos/CAGAACTGGAGTCCACTGGAGTCC | /5Phos/CGAAGGACTCCAGTGGACTCCAGT |
| ACTGTACTTCACTGTACTTC | /5Phos/CAGAACTGTACTTCACTGTACTTC | /5Phos/CGAAGAAGTACAGTGAAGTACAGT |
| AGAAGTCTGCAGAAGTCTGC | /5Phos/CAGAAGAAGTCTGCAGAAGTCTGC | /5Phos/CGAAGCAGACTTCTGCAGACTTCT |
| AGACGCGAGTAGACGCGAGT | /5Phos/CAGAAGACGCGAGTAGACGCGAGT | /5Phos/CGAAACTCGCGTCTACTCGCGTCT |
| AGACTGTGCTAGACTGTGCT | /5Phos/CAGAAGACTGTGCTAGACTGTGCT | /5Phos/CGAAAGCACAGTCTAGCACAGTCT |
| AGAGTACGCCAGAGTACGCC | /5Phos/CAGAAGAGTACGCCAGAGTACGCC | /5Phos/CGAAGGCGTACTCTGGCGTACTCT |
| AGATCGACTGAGATCGACTG | /5Phos/CAGAAGATCGACTGAGATCGACTG | /5Phos/CGAACAGTCGATCTCAGTCGATCT |
| AGATTCGGCCAGATTCGGCC | /5Phos/CAGAAGATTCGGCCAGATTCGGCC | /5Phos/CGAAGGCCGAATCTGGCCGAATCT |
| AGCAAGTGCTAGCAAGTGCT | /5Phos/CAGAAGCAAGTGCTAGCAAGTGCT | /5Phos/CGAAAGCACTTGCTAGCACTTGCT |
| AGCCATTGCGAGCCATTGCG | /5Phos/CAGAAGCCATTGCGAGCCATTGCG | /5Phos/CGAACGCAATGGCTCGCAATGGCT |
| AGCGGACAGTAGCGGACAGT | /5Phos/CAGAAGCGGACAGTAGCGGACAGT | /5Phos/CGAAACTGTCCGCTACTGTCCGCT |
| AGCGTAAGCGAGCGTAAGCG | /5Phos/CAGAAGCGTAAGCGAGCGTAAGCG | /5Phos/CGAACGCTTACGCTCGCTTACGCT |
| AGCTATTCTCAGCTATTCTC | /5Phos/CAGAAGCTATTCTCAGCTATTCTC | /5Phos/CGAAGAGAATAGCTGAGAATAGCT |
| AGCTTATACGAGCTTATACG | /5Phos/CAGAAGCTTATACGAGCTTATACG | /5Phos/CGAACGTATAAGCTCGTATAAGCT |
| AGGACAGATGAGGACAGATG | /5Phos/CAGAAGGACAGATGAGGACAGATG | /5Phos/CGAACATCTGTCCTCATCTGTCCT |
| AGGATCAGATAGGATCAGAT | /5Phos/CAGAAGGATCAGATAGGATCAGAT | /5Phos/CGAAATCTGATCCTATCTGATCCT |
| AGGCTGAAGGAGGCTGAAGG | /5Phos/CAGAAGGCTGAAGGAGGCTGAAGG | /5Phos/CGAACCTTCAGCCTCCTTCAGCCT |
| AGGTACGAGGAGGTACGAGG | /5Phos/CAGAAGGTACGAGGAGGTACGAGG | /5Phos/CGAACCTCGTACCTCCTCGTACCT |
| AGGTAGGCTCAGGTAGGCTC | /5Phos/CAGAAGGTAGGCTCAGGTAGGCTC | /5Phos/CGAAGAGCCTACCTGAGCCTACCT |
| AGGTGAACGGAGGTGAACGG | /5Phos/CAGAAGGTGAACGGAGGTGAACGG | /5Phos/CGAACCGTTCACCTCCGTTCACCT |
| AGGTGCCAATAGGTGCCAAT | /5Phos/CAGAAGGTGCCAATAGGTGCCAAT | /5Phos/CGAAATTGGCACCTATTGGCACCT |
| AGGTTCAACGAGGTTCAACG | /5Phos/CAGAAGGTTCAACGAGGTTCAACG | /5Phos/CGAACGTTGAACCTCGTTGAACCT |
| AGTCACTCGCAGTCACTCGC | /5Phos/CAGAAGTCACTCGCAGTCACTCGC | /5Phos/CGAAGCGAGTGACTGCGAGTGACT |
| AGTCGTGGTGAGTCGTGGTG | /5Phos/CAGAAGTCGTGGTGAGTCGTGGTG | /5Phos/CGAACACCACGACTCACCACGACT |
| AGTGGAGGAGAGTGGAGGAG | /5Phos/CAGAAGTGGAGGAGAGTGGAGGAG | /5Phos/CGAACTCCTCCACTCTCCTCCACT |
| AGTTGAGCGCAGTTGAGCGC | /5Phos/CAGAAGTTGAGCGCAGTTGAGCGC | /5Phos/CGAAGCGCTCAACTGCGCTCAACT |
| AGTTGATGGTAGTTGATGGT | /5Phos/CAGAAGTTGATGGTAGTTGATGGT | /5Phos/CGAAACCATCAACTACCATCAACT |
| ATAACACAGCATAACACAGC | /5Phos/CAGAATAACACAGCATAACACAGC | /5Phos/CGAAGCTGTGTTATGCTGTGTTAT |
| ATAAGGTCCGATAAGGTCCG | /5Phos/CAGAATAAGGTCCGATAAGGTCCG | /5Phos/CGAACGGACCTTATCGGACCTTAT |
| ATACAGTCAGATACAGTCAG | /5Phos/CAGAATACAGTCAGATACAGTCAG | /5Phos/CGAACTGACTGTATCTGACTGTAT |
| ATACCGGCCTATACCGGCCT | /5Phos/CAGAATACCGGCCTATACCGGCCT | /5Phos/CGAAAGGCCGGTATAGGCCGGTAT |
| ATAGCAGGATATAGCAGGAT | /5Phos/CAGAATAGCAGGATATAGCAGGAT | /5Phos/CGAAATCCTGCTATATCCTGCTAT |
| ATAGCGTTACATAGCGTTAC | /5Phos/CAGAATAGCGTTACATAGCGTTAC | /5Phos/CGAAGTAACGCTATGTAACGCTAT |
| ATAGTCCAACATAGTCCAAC | /5Phos/CAGAATAGTCCAACATAGTCCAAC | /5Phos/CGAAGTTGGACTATGTTGGACTAT |
| ATATCGGCGGATATCGGCGG | /5Phos/CAGAATATCGGCGGATATCGGCGG | /5Phos/CGAACCGCCGATATCCGCCGATAT |
| ATCCGTGATGATCCGTGATG | /5Phos/CAGAATCCGTGATGATCCGTGATG | /5Phos/CGAACATCACGGATCATCACGGAT |
| ATCGTACCGGATCGTACCGG | /5Phos/CAGAATCGTACCGGATCGTACCGG | /5Phos/CGAACCGGTACGATCCGGTACGAT |
| ATCGTTACTGATCGTTACTG | /5Phos/CAGAATCGTTACTGATCGTTACTG | /5Phos/CGAACAGTAACGATCAGTAACGAT |
| ATGAAGATGCATGAAGATGC | /5Phos/CAGAATGAAGATGCATGAAGATGC | /5Phos/CGAAGCATCTTCATGCATCTTCAT |
| ATGGCGTGGTATGGCGTGGT | /5Phos/CAGAATGGCGTGGTATGGCGTGGT | /5Phos/CGAAACCACGCCATACCACGCCAT |
| ATGTTGAGCGATGTTGAGCG | /5Phos/CAGAATGTTGAGCGATGTTGAGCG | /5Phos/CGAACGCTCAACATCGCTCAACAT |
| ATTAGCTGTCATTAGCTGTC | /5Phos/CAGAATTAGCTGTCATTAGCTGTC | /5Phos/CGAAGACAGCTAATGACAGCTAAT |
| ATTCGCGTGCATTCGCGTGC | /5Phos/CAGAATTCGCGTGCATTCGCGTGC | /5Phos/CGAAGCACGCGAATGCACGCGAAT |
| ATTGCGCTCCATTGCGCTCC | /5Phos/CAGAATTGCGCTCCATTGCGCTCC | /5Phos/CGAAGGAGCGCAATGGAGCGCAAT |
| ATTGTGTTGCATTGTGTTGC | /5Phos/CAGAATTGTGTTGCATTGTGTTGC | /5Phos/CGAAGCAACACAATGCAACACAAT |
| CAAGACGAATCAAGACGAAT | /5Phos/CAGACAAGACGAATCAAGACGAAT | /5Phos/CGAAATTCGTCTTGATTCGTCTTG |
| CACGCACTGTCACGCACTGT | /5Phos/CAGACACGCACTGTCACGCACTGT | /5Phos/CGAAACAGTGCGTGACAGTGCGTG |
| CACGGAGTAGCACGGAGTAG | /5Phos/CAGACACGGAGTAGCACGGAGTAG | /5Phos/CGAACTACTCCGTGCTACTCCGTG |
| CACGGTATCGCACGGTATCG | /5Phos/CAGACACGGTATCGCACGGTATCG | /5Phos/CGAACGATACCGTGCGATACCGTG |
| CACTCGGATTCACTCGGATT | /5Phos/CAGACACTCGGATTCACTCGGATT | /5Phos/CGAAAATCCGAGTGAATCCGAGTG |
| CACTTGAGTACACTTGAGTA | /5Phos/CAGACACTTGAGTACACTTGAGTA | /5Phos/CGAATACTCAAGTGTACTCAAGTG |
| CAGCCTCAGTCAGCCTCAGT | /5Phos/CAGACAGCCTCAGTCAGCCTCAGT | /5Phos/CGAAACTGAGGCTGACTGAGGCTG |
| CAGCTCCAAGCAGCTCCAAG | /5Phos/CAGACAGCTCCAAGCAGCTCCAAG | /5Phos/CGAACTTGGAGCTGCTTGGAGCTG |
| CATCGAGGAGCATCGAGGAG | /5Phos/CAGACATCGAGGAGCATCGAGGAG | /5Phos/CGAACTCCTCGATGCTCCTCGATG |
| CATTGGCCGTCATTGGCCGT | /5Phos/CAGACATTGGCCGTCATTGGCCGT | /5Phos/CGAAACGGCCAATGACGGCCAATG |
| CCAGAACTGGCCAGAACTGG | /5Phos/CAGACCAGAACTGGCCAGAACTGG | /5Phos/CGAACCAGTTCTGGCCAGTTCTGG |
| CCAGATACGGCCAGATACGG | /5Phos/CAGACCAGATACGGCCAGATACGG | /5Phos/CGAACCGTATCTGGCCGTATCTGG |
| CCAGGATCCACCAGGATCCA | /5Phos/CAGACCAGGATCCACCAGGATCCA | /5Phos/CGAATGGATCCTGGTGGATCCTGG |
| CCAGGATGTTCCAGGATGTT | /5Phos/CAGACCAGGATGTTCCAGGATGTT | /5Phos/CGAAAACATCCTGGAACATCCTGG |
| CCGAGCATGTCCGAGCATGT | /5Phos/CAGACCGAGCATGTCCGAGCATGT | /5Phos/CGAAACATGCTCGGACATGCTCGG |
| CCGGAGTGTTCCGGAGTGTT | /5Phos/CAGACCGGAGTGTTCCGGAGTGTT | /5Phos/CGAAAACACTCCGGAACACTCCGG |
| CCGGTACCATCCGGTACCAT | /5Phos/CAGACCGGTACCATCCGGTACCAT | /5Phos/CGAAATGGTACCGGATGGTACCGG |
| CCGTCTAAGGCCGTCTAAGG | /5Phos/CAGACCGTCTAAGGCCGTCTAAGG | /5Phos/CGAACCTTAGACGGCCTTAGACGG |
| CCTCCATTAACCTCCATTAA | /5Phos/CAGACCTCCATTAACCTCCATTAA | /5Phos/CGAATTAATGGAGGTTAATGGAGG |
| CCTCCTGACTCCTCCTGACT | /5Phos/CAGACCTCCTGACTCCTCCTGACT | /5Phos/CGAAAGTCAGGAGGAGTCAGGAGG |
| CCTCTGCTCTCCTCTGCTCT | /5Phos/CAGACCTCTGCTCTCCTCTGCTCT | /5Phos/CGAAAGAGCAGAGGAGAGCAGAGG |
| CCTGCCTTGTCCTGCCTTGT | /5Phos/CAGACCTGCCTTGTCCTGCCTTGT | /5Phos/CGAAACAAGGCAGGACAAGGCAGG |
| CCTGGCCATTCCTGGCCATT | /5Phos/CAGACCTGGCCATTCCTGGCCATT | /5Phos/CGAAAATGGCCAGGAATGGCCAGG |
| CCTTAACGCGCCTTAACGCG | /5Phos/CAGACCTTAACGCGCCTTAACGCG | /5Phos/CGAACGCGTTAAGGCGCGTTAAGG |
| CGACCTGTCTCGACCTGTCT | /5Phos/CAGACGACCTGTCTCGACCTGTCT | /5Phos/CGAAAGACAGGTCGAGACAGGTCG |
| CGCGTTACGTCGCGTTACGT | /5Phos/CAGACGCGTTACGTCGCGTTACGT | /5Phos/CGAAACGTAACGCGACGTAACGCG |
| CGGCAGTTCACGGCAGTTCA | /5Phos/CAGACGGCAGTTCACGGCAGTTCA | /5Phos/CGAATGAACTGCCGTGAACTGCCG |
| CGGCCATTAGCGGCCATTAG | /5Phos/CAGACGGCCATTAGCGGCCATTAG | /5Phos/CGAACTAATGGCCGCTAATGGCCG |
| CGTATTGCATCGTATTGCAT | /5Phos/CAGACGTATTGCATCGTATTGCAT | /5Phos/CGAAATGCAATACGATGCAATACG |
| CTACGACCGTCTACGACCGT | /5Phos/CAGACTACGACCGTCTACGACCGT | /5Phos/CGAAACGGTCGTAGACGGTCGTAG |
| CTAGGAAGGTCTAGGAAGGT | /5Phos/CAGACTAGGAAGGTCTAGGAAGGT | /5Phos/CGAAACCTTCCTAGACCTTCCTAG |
| CTAGTCCTGTCTAGTCCTGT | /5Phos/CAGACTAGTCCTGTCTAGTCCTGT | /5Phos/CGAAACAGGACTAGACAGGACTAG |
| CTAGTGGAGGCTAGTGGAGG | /5Phos/CAGACTAGTGGAGGCTAGTGGAGG | /5Phos/CGAACCTCCACTAGCCTCCACTAG |
| CTCGCAGAGTCTCGCAGAGT | /5Phos/CAGACTCGCAGAGTCTCGCAGAGT | /5Phos/CGAAACTCTGCGAGACTCTGCGAG |
| CTCGCTTCGTCTCGCTTCGT | /5Phos/CAGACTCGCTTCGTCTCGCTTCGT | /5Phos/CGAAACGAAGCGAGACGAAGCGAG |
| CTCGTTAGCGCTCGTTAGCG | /5Phos/CAGACTCGTTAGCGCTCGTTAGCG | /5Phos/CGAACGCTAACGAGCGCTAACGAG |
| CTCTTCCAAGCTCTTCCAAG | /5Phos/CAGACTCTTCCAAGCTCTTCCAAG | /5Phos/CGAACTTGGAAGAGCTTGGAAGAG |
| CTGCTTCAATCTGCTTCAAT | /5Phos/CAGACTGCTTCAATCTGCTTCAAT | /5Phos/CGAAATTGAAGCAGATTGAAGCAG |
| CTGGTATCAACTGGTATCAA | /5Phos/CAGACTGGTATCAACTGGTATCAA | /5Phos/CGAATTGATACCAGTTGATACCAG |
| CTGTCTTCGGCTGTCTTCGG | /5Phos/CAGACTGTCTTCGGCTGTCTTCGG | /5Phos/CGAACCGAAGACAGCCGAAGACAG |
| CTTCATGACGCTTCATGACG | /5Phos/CAGACTTCATGACGCTTCATGACG | /5Phos/CGAACGTCATGAAGCGTCATGAAG |
| CTTCGGCAGTCTTCGGCAGT | /5Phos/CAGACTTCGGCAGTCTTCGGCAGT | /5Phos/CGAAACTGCCGAAGACTGCCGAAG |
| CTTGACCGGTCTTGACCGGT | /5Phos/CAGACTTGACCGGTCTTGACCGGT | /5Phos/CGAAACCGGTCAAGACCGGTCAAG |
| CTTGCCTATTCTTGCCTATT | /5Phos/CAGACTTGCCTATTCTTGCCTATT | /5Phos/CGAAAATAGGCAAGAATAGGCAAG |
| GAACTTGTGAGAACTTGTGA | /5Phos/CAGAGAACTTGTGAGAACTTGTGA | /5Phos/CGAATCACAAGTTCTCACAAGTTC |
| GAAGCATTCTGAAGCATTCT | /5Phos/CAGAGAAGCATTCTGAAGCATTCT | /5Phos/CGAAAGAATGCTTCAGAATGCTTC |
| GAATCCATTCGAATCCATTC | /5Phos/CAGAGAATCCATTCGAATCCATTC | /5Phos/CGAAGAATGGATTCGAATGGATTC |
| GACGCCTGTTGACGCCTGTT | /5Phos/CAGAGACGCCTGTTGACGCCTGTT | /5Phos/CGAAAACAGGCGTCAACAGGCGTC |
| GACGTAGGACGACGTAGGAC | /5Phos/CAGAGACGTAGGACGACGTAGGAC | /5Phos/CGAAGTCCTACGTCGTCCTACGTC |
| GACTAATGGTGACTAATGGT | /5Phos/CAGAGACTAATGGTGACTAATGGT | /5Phos/CGAAACCATTAGTCACCATTAGTC |
| GAGCCTCCTTGAGCCTCCTT | /5Phos/CAGAGAGCCTCCTTGAGCCTCCTT | /5Phos/CGAAAAGGAGGCTCAAGGAGGCTC |
| GAGCGTCTACGAGCGTCTAC | /5Phos/CAGAGAGCGTCTACGAGCGTCTAC | /5Phos/CGAAGTAGACGCTCGTAGACGCTC |
| GAGGATAGGCGAGGATAGGC | /5Phos/CAGAGAGGATAGGCGAGGATAGGC | /5Phos/CGAAGCCTATCCTCGCCTATCCTC |
| GAGTGCCATCGAGTGCCATC | /5Phos/CAGAGAGTGCCATCGAGTGCCATC | /5Phos/CGAAGATGGCACTCGATGGCACTC |
| GAGTGGATCTGAGTGGATCT | /5Phos/CAGAGAGTGGATCTGAGTGGATCT | /5Phos/CGAAAGATCCACTCAGATCCACTC |
| GAGTGGTAGCGAGTGGTAGC | /5Phos/CAGAGAGTGGTAGCGAGTGGTAGC | /5Phos/CGAAGCTACCACTCGCTACCACTC |
| GAGTTAGAGAGAGTTAGAGA | /5Phos/CAGAGAGTTAGAGAGAGTTAGAGA | /5Phos/CGAATCTCTAACTCTCTCTAACTC |
| GCACATCTGCGCACATCTGC | /5Phos/CAGAGCACATCTGCGCACATCTGC | /5Phos/CGAAGCAGATGTGCGCAGATGTGC |
| GCACCATTACGCACCATTAC | /5Phos/CAGAGCACCATTACGCACCATTAC | /5Phos/CGAAGTAATGGTGCGTAATGGTGC |
| GCAGCCTATTGCAGCCTATT | /5Phos/CAGAGCAGCCTATTGCAGCCTATT | /5Phos/CGAAAATAGGCTGCAATAGGCTGC |
| GCAGTATCAAGCAGTATCAA | /5Phos/CAGAGCAGTATCAAGCAGTATCAA | /5Phos/CGAATTGATACTGCTTGATACTGC |
| GCCGTCGTTAGCCGTCGTTA | /5Phos/CAGAGCCGTCGTTAGCCGTCGTTA | /5Phos/CGAATAACGACGGCTAACGACGGC |
| GCCTGAGCTAGCCTGAGCTA | /5Phos/CAGAGCCTGAGCTAGCCTGAGCTA | /5Phos/CGAATAGCTCAGGCTAGCTCAGGC |
| GCGCAAGCAAGCGCAAGCAA | /5Phos/CAGAGCGCAAGCAAGCGCAAGCAA | /5Phos/CGAATTGCTTGCGCTTGCTTGCGC |
| GCGTTACGACGCGTTACGAC | /5Phos/CAGAGCGTTACGACGCGTTACGAC | /5Phos/CGAAGTCGTAACGCGTCGTAACGC |
| GCGTTGGATCGCGTTGGATC | /5Phos/CAGAGCGTTGGATCGCGTTGGATC | /5Phos/CGAAGATCCAACGCGATCCAACGC |
| GCTAGTCGCAGCTAGTCGCA | /5Phos/CAGAGCTAGTCGCAGCTAGTCGCA | /5Phos/CGAATGCGACTAGCTGCGACTAGC |
| GCTCACTACCGCTCACTACC | /5Phos/CAGAGCTCACTACCGCTCACTACC | /5Phos/CGAAGGTAGTGAGCGGTAGTGAGC |
| GCTCGAATTAGCTCGAATTA | /5Phos/CAGAGCTCGAATTAGCTCGAATTA | /5Phos/CGAATAATTCGAGCTAATTCGAGC |
| GCTCGATTCCGCTCGATTCC | /5Phos/CAGAGCTCGATTCCGCTCGATTCC | /5Phos/CGAAGGAATCGAGCGGAATCGAGC |
| GCTGAGGATCGCTGAGGATC | /5Phos/CAGAGCTGAGGATCGCTGAGGATC | /5Phos/CGAAGATCCTCAGCGATCCTCAGC |
| GCTTCATTCTGCTTCATTCT | /5Phos/CAGAGCTTCATTCTGCTTCATTCT | /5Phos/CGAAAGAATGAAGCAGAATGAAGC |
| GCTTGCTATTGCTTGCTATT | /5Phos/CAGAGCTTGCTATTGCTTGCTATT | /5Phos/CGAAAATAGCAAGCAATAGCAAGC |
| GCTTGGTTGCGCTTGGTTGC | /5Phos/TTCGGCTTGGTTGCGCTTGGTTGC | /5Phos/GTCAGCAACCAAGCGCAACCAAGC |
| GGAGTGGTTCGGAGTGGTTC | /5Phos/TTCGGGAGTGGTTCGGAGTGGTTC | /5Phos/GTCAGAACCACTCCGAACCACTCC |
| GGATAATACCGGATAATACC | /5Phos/TTCGGGATAATACCGGATAATACC | /5Phos/GTCAGGTATTATCCGGTATTATCC |
| GGATCGTGGTGGATCGTGGT | /5Phos/TTCGGGATCGTGGTGGATCGTGGT | /5Phos/GTCAACCACGATCCACCACGATCC |
| GGATGATTGTGGATGATTGT | /5Phos/TTCGGGATGATTGTGGATGATTGT | /5Phos/GTCAACAATCATCCACAATCATCC |
| GGATGCGTTCGGATGCGTTC | /5Phos/TTCGGGATGCGTTCGGATGCGTTC | /5Phos/GTCAGAACGCATCCGAACGCATCC |
| GGCGAATGTCGGCGAATGTC | /5Phos/TTCGGGCGAATGTCGGCGAATGTC | /5Phos/GTCAGACATTCGCCGACATTCGCC |
| GGCGATTGGTGGCGATTGGT | /5Phos/TTCGGGCGATTGGTGGCGATTGGT | /5Phos/GTCAACCAATCGCCACCAATCGCC |
| GGCGGTATCAGGCGGTATCA | /5Phos/TTCGGGCGGTATCAGGCGGTATCA | /5Phos/GTCATGATACCGCCTGATACCGCC |
| GGCTATCCACGGCTATCCAC | /5Phos/TTCGGGCTATCCACGGCTATCCAC | /5Phos/GTCAGTGGATAGCCGTGGATAGCC |
| GGCTATTACAGGCTATTACA | /5Phos/TTCGGGCTATTACAGGCTATTACA | /5Phos/GTCATGTAATAGCCTGTAATAGCC |
| GGCTGAACTCGGCTGAACTC | /5Phos/TTCGGGCTGAACTCGGCTGAACTC | /5Phos/GTCAGAGTTCAGCCGAGTTCAGCC |
| GGTACAGTCAGGTACAGTCA | /5Phos/TTCGGGTACAGTCAGGTACAGTCA | /5Phos/GTCATGACTGTACCTGACTGTACC |
| GGTCGAACCTGGTCGAACCT | /5Phos/TTCGGGTCGAACCTGGTCGAACCT | /5Phos/GTCAAGGTTCGACCAGGTTCGACC |
| GGTCTCTCGTGGTCTCTCGT | /5Phos/TTCGGGTCTCTCGTGGTCTCTCGT | /5Phos/GTCAACGAGAGACCACGAGAGACC |
| GGTGCTTGTCGGTGCTTGTC | /5Phos/TTCGGGTGCTTGTCGGTGCTTGTC | /5Phos/GTCAGACAAGCACCGACAAGCACC |
| GGTTCCACTTGGTTCCACTT | /5Phos/TTCGGGTTCCACTTGGTTCCACTT | /5Phos/GTCAAAGTGGAACCAAGTGGAACC |
| GGTTGCATCCGGTTGCATCC | /5Phos/TTCGGGTTGCATCCGGTTGCATCC | /5Phos/GTCAGGATGCAACCGGATGCAACC |
| GGTTGGACGTGGTTGGACGT | /5Phos/TTCGGGTTGGACGTGGTTGGACGT | /5Phos/GTCAACGTCCAACCACGTCCAACC |
| GTAAGAGCAAGTAAGAGCAA | /5Phos/TTCGGTAAGAGCAAGTAAGAGCAA | /5Phos/GTCATTGCTCTTACTTGCTCTTAC |
| GTAAGGCTTAGTAAGGCTTA | /5Phos/TTCGGTAAGGCTTAGTAAGGCTTA | /5Phos/GTCATAAGCCTTACTAAGCCTTAC |
| GTAGTCCTCAGTAGTCCTCA | /5Phos/TTCGGTAGTCCTCAGTAGTCCTCA | /5Phos/GTCATGAGGACTACTGAGGACTAC |
| GTCCGGTTCTGTCCGGTTCT | /5Phos/TTCGGTCCGGTTCTGTCCGGTTCT | /5Phos/GTCAAGAACCGGACAGAACCGGAC |
| GTCCTACAGCGTCCTACAGC | /5Phos/TTCGGTCCTACAGCGTCCTACAGC | /5Phos/GTCAGCTGTAGGACGCTGTAGGAC |
| GTCGGATTAAGTCGGATTAA | /5Phos/TTCGGTCGGATTAAGTCGGATTAA | /5Phos/GTCATTAATCCGACTTAATCCGAC |
| GTGAACAGAAGTGAACAGAA | /5Phos/TTCGGTGAACAGAAGTGAACAGAA | /5Phos/GTCATTCTGTTCACTTCTGTTCAC |
| GTGCCTACACGTGCCTACAC | /5Phos/TTCGGTGCCTACACGTGCCTACAC | /5Phos/GTCAGTGTAGGCACGTGTAGGCAC |
| GTGTATCGGAGTGTATCGGA | /5Phos/TTCGGTGTATCGGAGTGTATCGGA | /5Phos/GTCATCCGATACACTCCGATACAC |
| GTGTCCATGAGTGTCCATGA | /5Phos/TTCGGTGTCCATGAGTGTCCATGA | /5Phos/GTCATCATGGACACTCATGGACAC |
| GTGTCTAATCGTGTCTAATC | /5Phos/TTCGGTGTCTAATCGTGTCTAATC | /5Phos/GTCAGATTAGACACGATTAGACAC |
| GTGTTCCTGCGTGTTCCTGC | /5Phos/TTCGGTGTTCCTGCGTGTTCCTGC | /5Phos/GTCAGCAGGAACACGCAGGAACAC |
| GTGTTCTGCTGTGTTCTGCT | /5Phos/TTCGGTGTTCTGCTGTGTTCTGCT | /5Phos/GTCAAGCAGAACACAGCAGAACAC |
| GTTAAGAGGAGTTAAGAGGA | /5Phos/TTCGGTTAAGAGGAGTTAAGAGGA | /5Phos/GTCATCCTCTTAACTCCTCTTAAC |
| GTTAATGCGTGTTAATGCGT | /5Phos/TTCGGTTAATGCGTGTTAATGCGT | /5Phos/GTCAACGCATTAACACGCATTAAC |
| GTTAGGCTGTGTTAGGCTGT | /5Phos/TTCGGTTAGGCTGTGTTAGGCTGT | /5Phos/GTCAACAGCCTAACACAGCCTAAC |
| GTTCATTGGAGTTCATTGGA | /5Phos/TTCGGTTCATTGGAGTTCATTGGA | /5Phos/GTCATCCAATGAACTCCAATGAAC |
| GTTCGGACCAGTTCGGACCA | /5Phos/TTCGGTTCGGACCAGTTCGGACCA | /5Phos/GTCATGGTCCGAACTGGTCCGAAC |
| GTTGGCCAGTGTTGGCCAGT | /5Phos/TTCGGTTGGCCAGTGTTGGCCAGT | /5Phos/GTCAACTGGCCAACACTGGCCAAC |
| GTTGGTAGTTGTTGGTAGTT | /5Phos/TTCGGTTGGTAGTTGTTGGTAGTT | /5Phos/GTCAAACTACCAACAACTACCAAC |
| TAACACGACATAACACGACA | /5Phos/TTCGTAACACGACATAACACGACA | /5Phos/GTCATGTCGTGTTATGTCGTGTTA |
| TAAGAGAGCATAAGAGAGCA | /5Phos/TTCGTAAGAGAGCATAAGAGAGCA | /5Phos/GTCATGCTCTCTTATGCTCTCTTA |
| TAAGAGGCGGTAAGAGGCGG | /5Phos/TTCGTAAGAGGCGGTAAGAGGCGG | /5Phos/GTCACCGCCTCTTACCGCCTCTTA |
| TAAGGAATGGTAAGGAATGG | /5Phos/TTCGTAAGGAATGGTAAGGAATGG | /5Phos/GTCACCATTCCTTACCATTCCTTA |
| TAATGAGCACTAATGAGCAC | /5Phos/TTCGTAATGAGCACTAATGAGCAC | /5Phos/GTCAGTGCTCATTAGTGCTCATTA |
| TACACTGGTCTACACTGGTC | /5Phos/TTCGTACACTGGTCTACACTGGTC | /5Phos/GTCAGACCAGTGTAGACCAGTGTA |
| TACAGCGCAATACAGCGCAA | /5Phos/TTCGTACAGCGCAATACAGCGCAA | /5Phos/GTCATTGCGCTGTATTGCGCTGTA |
| TACAGGTTAGTACAGGTTAG | /5Phos/TTCGTACAGGTTAGTACAGGTTAG | /5Phos/GTCACTAACCTGTACTAACCTGTA |
| TACATTACCGTACATTACCG | /5Phos/TTCGTACATTACCGTACATTACCG | /5Phos/GTCACGGTAATGTACGGTAATGTA |
| TACCAATCTCTACCAATCTC | /5Phos/TTCGTACCAATCTCTACCAATCTC | /5Phos/GTCAGAGATTGGTAGAGATTGGTA |
| TACCGGAGAGTACCGGAGAG | /5Phos/TTCGTACCGGAGAGTACCGGAGAG | /5Phos/GTCACTCTCCGGTACTCTCCGGTA |
| TACCGGCTTCTACCGGCTTC | /5Phos/TTCGTACCGGCTTCTACCGGCTTC | /5Phos/GTCAGAAGCCGGTAGAAGCCGGTA |
| TACCTACATGTACCTACATG | /5Phos/TTCGTACCTACATGTACCTACATG | /5Phos/GTCACATGTAGGTACATGTAGGTA |
| TACGATTACGTACGATTACG | /5Phos/TTCGTACGATTACGTACGATTACG | /5Phos/GTCACGTAATCGTACGTAATCGTA |
| TACGCAGAGGTACGCAGAGG | /5Phos/TTCGTACGCAGAGGTACGCAGAGG | /5Phos/GTCACCTCTGCGTACCTCTGCGTA |
| TACTCAACGATACTCAACGA | /5Phos/TTCGTACTCAACGATACTCAACGA | /5Phos/GTCATCGTTGAGTATCGTTGAGTA |
| TAGACCAACCTAGACCAACC | /5Phos/TTCGTAGACCAACCTAGACCAACC | /5Phos/GTCAGGTTGGTCTAGGTTGGTCTA |
| TAGACCTCCGTAGACCTCCG | /5Phos/TTCGTAGACCTCCGTAGACCTCCG | /5Phos/GTCACGGAGGTCTACGGAGGTCTA |
| TAGCAGTTGATAGCAGTTGA | /5Phos/TTCGTAGCAGTTGATAGCAGTTGA | /5Phos/GTCATCAACTGCTATCAACTGCTA |
| TAGCGCAAGCTAGCGCAAGC | /5Phos/TTCGTAGCGCAAGCTAGCGCAAGC | /5Phos/GTCAGCTTGCGCTAGCTTGCGCTA |
| TAGCTATACCTAGCTATACC | /5Phos/TTCGTAGCTATACCTAGCTATACC | /5Phos/GTCAGGTATAGCTAGGTATAGCTA |
| TAGCTTGCGCTAGCTTGCGC | /5Phos/TTCGTAGCTTGCGCTAGCTTGCGC | /5Phos/GTCAGCGCAAGCTAGCGCAAGCTA |
| TAGGAATCCATAGGAATCCA | /5Phos/TTCGTAGGAATCCATAGGAATCCA | /5Phos/GTCATGGATTCCTATGGATTCCTA |
| TAGGCCGTTCTAGGCCGTTC | /5Phos/TTCGTAGGCCGTTCTAGGCCGTTC | /5Phos/GTCAGAACGGCCTAGAACGGCCTA |
| TAGGTCACTATAGGTCACTA | /5Phos/TTCGTAGGTCACTATAGGTCACTA | /5Phos/GTCATAGTGACCTATAGTGACCTA |
| TAGTCGCGTCTAGTCGCGTC | /5Phos/TTCGTAGTCGCGTCTAGTCGCGTC | /5Phos/GTCAGACGCGACTAGACGCGACTA |
| TATACGGCTATATACGGCTA | /5Phos/TTCGTATACGGCTATATACGGCTA | /5Phos/GTCATAGCCGTATATAGCCGTATA |
| TATCGCGGCATATCGCGGCA | /5Phos/TTCGTATCGCGGCATATCGCGGCA | /5Phos/GTCATGCCGCGATATGCCGCGATA |
| TATGGAGCAATATGGAGCAA | /5Phos/TTCGTATGGAGCAATATGGAGCAA | /5Phos/GTCATTGCTCCATATTGCTCCATA |
| TATGGCGTGGTATGGCGTGG | /5Phos/TTCGTATGGCGTGGTATGGCGTGG | /5Phos/GTCACCACGCCATACCACGCCATA |
| TATGTTCAGGTATGTTCAGG | /5Phos/TTCGTATGTTCAGGTATGTTCAGG | /5Phos/GTCACCTGAACATACCTGAACATA |
| TATTCCTGTCTATTCCTGTC | /5Phos/TTCGTATTCCTGTCTATTCCTGTC | /5Phos/GTCAGACAGGAATAGACAGGAATA |
| TCAAGAGATCTCAAGAGATC | /5Phos/TTCGTCAAGAGATCTCAAGAGATC | /5Phos/GTCAGATCTCTTGAGATCTCTTGA |
| TCACTACCAATCACTACCAA | /5Phos/TTCGTCACTACCAATCACTACCAA | /5Phos/GTCATTGGTAGTGATTGGTAGTGA |
| TCAGTCTGCGTCAGTCTGCG | /5Phos/TTCGTCAGTCTGCGTCAGTCTGCG | /5Phos/GTCACGCAGACTGACGCAGACTGA |
| TCAGTTAAGCTCAGTTAAGC | /5Phos/TTCGTCAGTTAAGCTCAGTTAAGC | /5Phos/GTCAGCTTAACTGAGCTTAACTGA |
| TCCAGAGTGGTCCAGAGTGG | /5Phos/TTCGTCCAGAGTGGTCCAGAGTGG | /5Phos/GTCACCACTCTGGACCACTCTGGA |
| TCCAGTCGTCTCCAGTCGTC | /5Phos/TTCGTCCAGTCGTCTCCAGTCGTC | /5Phos/GTCAGACGACTGGAGACGACTGGA |
| TCCGACGTTGTCCGACGTTG | /5Phos/TTCGTCCGACGTTGTCCGACGTTG | /5Phos/GTCACAACGTCGGACAACGTCGGA |
| TCCTACCGACTCCTACCGAC | /5Phos/TTCGTCCTACCGACTCCTACCGAC | /5Phos/GTCAGTCGGTAGGAGTCGGTAGGA |
| TCCTTCCTCCTCCTTCCTCC | /5Phos/TTCGTCCTTCCTCCTCCTTCCTCC | /5Phos/GTCAGGAGGAAGGAGGAGGAAGGA |
| TCGAGAGCCATCGAGAGCCA | /5Phos/TTCGTCGAGAGCCATCGAGAGCCA | /5Phos/GTCATGGCTCTCGATGGCTCTCGA |
| TCGCACAGACTCGCACAGAC | /5Phos/TTCGTCGCACAGACTCGCACAGAC | /5Phos/GTCAGTCTGTGCGAGTCTGTGCGA |
| TCGCCGTTAGTCGCCGTTAG | /5Phos/TTCGTCGCCGTTAGTCGCCGTTAG | /5Phos/GTCACTAACGGCGACTAACGGCGA |
| TCGGCACAACTCGGCACAAC | /5Phos/TTCGTCGGCACAACTCGGCACAAC | /5Phos/GTCAGTTGTGCCGAGTTGTGCCGA |
| TCGGCAGTTGTCGGCAGTTG | /5Phos/TTCGTCGGCAGTTGTCGGCAGTTG | /5Phos/GTCACAACTGCCGACAACTGCCGA |
| TCGGTCACACTCGGTCACAC | /5Phos/TTCGTCGGTCACACTCGGTCACAC | /5Phos/GTCAGTGTGACCGAGTGTGACCGA |
| TCGTGCTAGCTCGTGCTAGC | /5Phos/TTCGTCGTGCTAGCTCGTGCTAGC | /5Phos/GTCAGCTAGCACGAGCTAGCACGA |
| TCTAGCCTAATCTAGCCTAA | /5Phos/TTCGTCTAGCCTAATCTAGCCTAA | /5Phos/GTCATTAGGCTAGATTAGGCTAGA |
| TCTCACTGCGTCTCACTGCG | /5Phos/TTCGTCTCACTGCGTCTCACTGCG | /5Phos/GTCACGCAGTGAGACGCAGTGAGA |
| TCTCCGGCAATCTCCGGCAA | /5Phos/TTCGTCTCCGGCAATCTCCGGCAA | /5Phos/GTCATTGCCGGAGATTGCCGGAGA |
| TCTCGCTCTCTCTCGCTCTC | /5Phos/TTCGTCTCGCTCTCTCTCGCTCTC | /5Phos/GTCAGAGAGCGAGAGAGAGCGAGA |
| TCTGTCGCAATCTGTCGCAA | /5Phos/TTCGTCTGTCGCAATCTGTCGCAA | /5Phos/GTCATTGCGACAGATTGCGACAGA |
| TCTTAACCTCTCTTAACCTC | /5Phos/TTCGTCTTAACCTCTCTTAACCTC | /5Phos/GTCAGAGGTTAAGAGAGGTTAAGA |
| TGACACATGATGACACATGA | /5Phos/TTCGTGACACATGATGACACATGA | /5Phos/GTCATCATGTGTCATCATGTGTCA |
| TGAGAGTGAATGAGAGTGAA | /5Phos/TTCGTGAGAGTGAATGAGAGTGAA | /5Phos/GTCATTCACTCTCATTCACTCTCA |
| TGAGTGGCTGTGAGTGGCTG | /5Phos/TTCGTGAGTGGCTGTGAGTGGCTG | /5Phos/GTCACAGCCACTCACAGCCACTCA |
| TGATTGACCATGATTGACCA | /5Phos/TTCGTGATTGACCATGATTGACCA | /5Phos/GTCATGGTCAATCATGGTCAATCA |
| TGCAGTCGCATGCAGTCGCA | /5Phos/TTCGTGCAGTCGCATGCAGTCGCA | /5Phos/GTCATGCGACTGCATGCGACTGCA |
| TGCCATGAGGTGCCATGAGG | /5Phos/TTCGTGCAATGAGGTGCCATGAGG | /5Phos/GTCACCTCATGGCACCTCATGGCA |
| TGCCTAACGCTGCCTAACGC | /5Phos/TTCGTGCCTAACGCTGCCTAACGC | /5Phos/GTCAGCGTTAGGCAGCGTTAGGCA |
| TGCCTATAACTGCCTATAAC | /5Phos/TTCGTGCCTATAACTGCCTATAAC | /5Phos/GTCAGTTATAGGCAGTTATAGGCA |
| TGCGAGAGAGTGCGAGAGAG | /5Phos/TTCGTGCGAGAGAGTGCGAGAGAG | /5Phos/GTCACTCTCTCGCACTCTCTCGCA |
| TGCGCGATCATGCGCGATCA | /5Phos/TTCGTGCGCGATCATGCGCGATCA | /5Phos/GTCATGATCGCGCATGATCGCGCA |
| TGCGGAGTGATGCGGAGTGA | /5Phos/TTCGTGCGGAGTGATGCGGAGTGA | /5Phos/GTCATCACTCCGCATCACTCCGCA |
| TGCGGATTCCTGCGGATTCC | /5Phos/TTCGTGCGGATTCCTGCGGATTCC | /5Phos/GTCAGGAATCCGCAGGAATCCGCA |
| TGCGGCTACATGCGGCTACA | /5Phos/TTCGTGCGGCTACATGCGGCTACA | /5Phos/GTCATGTAGCCGCATGTAGCCGCA |
| TGCGTAACAATGCGTAACAA | /5Phos/TTCGTGCGTAACAATGCGTAACAA | /5Phos/GTCATTGTTACGCATTGTTACGCA |
| TGCGTCCTCATGCGTCCTCA | /5Phos/TTCGTGCGTCCTCATGCGTCCTCA | /5Phos/GTCATGAGGACGCATGAGGACGCA |
| TGCGTTCAGCTGCGTTCAGC | /5Phos/TTCGTGCGTTCAGCTGCGTTCAGC | /5Phos/GTCAGCTGAACGCAGCTGAACGCA |
| TGCTTCAGCGTGCTTCAGCG | /5Phos/TTCGTGCTTCAGCGTGCTTCAGCG | /5Phos/GTCACGCTGAAGCACGCTGAAGCA |
| TGCTTGCCTCTGCTTGCCTC | /5Phos/TTCGTGCTTGCCTCTGCTTGCCTC | /5Phos/GTCAGAGGCAAGCAGAGGCAAGCA |
| TGGAACTGGCTGGAACTGGC | /5Phos/TTCGTGGAACTGGCTGGAACTGGC | /5Phos/GTCAGCCAGTTCCAGCCAGTTCCA |
| TGTATTGGAGTGTATTGGAG | /5Phos/TTCGTGTATTGGAGTGTATTGGAG | /5Phos/GTCACTCCAATACACTCCAATACA |
| TGTGGAACGGTGTGGAACGG | /5Phos/TTCGTGTGGAACGGTGTGGAACGG | /5Phos/GTCACCGTTCCACACCGTTCCACA |
| TGTGGAGTCGTGTGGAGTCG | /5Phos/TTCGTGTGGAGTCGTGTGGAGTCG | /5Phos/GTCACGACTCCACACGACTCCACA |
| TGTGTGGCCATGTGTGGCCA | /5Phos/TTCGTGTGTGGCCATGTGTGGCCA | /5Phos/GTCATGGCCACACATGGCCACACA |
| TGTTAAGAGGTGTTAAGAGG | /5Phos/TTCGTGTTAAGAGGTGTTAAGAGG | /5Phos/GTCACCTCTTAACACCTCTTAACA |
| TGTTACTCACTGTTACTCAC | /5Phos/TTCGTGTTACTCACTGTTACTCAC | /5Phos/GTCAGTGAGTAACAGTGAGTAACA |
| TGTTCGCAGCTGTTCGCAGC | /5Phos/TTCGTGTTCGCAGCTGTTCGCAGC | /5Phos/GTCAGCTGCGAACAGCTGCGAACA |
| TGTTGGCATATGTTGGCATA | /5Phos/TTCGTGTTGGCATATGTTGGCATA | /5Phos/GTCATATGCCAACATATGCCAACA |
| TTAAGTGCAGTTAAGTGCAG | /5Phos/TTCGTTAAGTGCAGTTAAGTGCAG | /5Phos/GTCACTGCACTTAACTGCACTTAA |
| TTAGAGATCCTTAGAGATCC | /5Phos/TTCGTTAGAGATCCTTAGAGATCC | /5Phos/GTCAGGATCTCTAAGGATCTCTAA |
| TTAGGTCAGATTAGGTCAGA | /5Phos/TTCGTTAGGTCAGATTAGGTCAGA | /5Phos/GTCATCTGACCTAATCTGACCTAA |
| TTATCTCCTGTTATCTCCTG | /5Phos/TTCGTTATCTCCTGTTATCTCCTG | /5Phos/GTCACAGGAGATAACAGGAGATAA |
| TTCAGTGTCGTTCAGTGTCG | /5Phos/TTCGTTCAGTGTCGTTCAGTGTCG | /5Phos/GTCACGACACTGAACGACACTGAA |
| TTCCAAGAAGTTCCAAGAAG | /5Phos/TTCGTTCCAAGAAGTTCCAAGAAG | /5Phos/GTCACTTCTTGGAACTTCTTGGAA |
| TTCCACCGCATTCCACCGCA | /5Phos/TTCGTTCCACCGCATTCCACCGCA | /5Phos/GTCATGCGGTGGAATGCGGTGGAA |
| TTCCACTATCTTCCACTATC | /5Phos/TTCGTTCCACTATCTTCCACTATC | /5Phos/GTCAGATAGTGGAAGATAGTGGAA |
| TTCCACTTGATTCCACTTGA | /5Phos/TTCGTTCCACTTGATTCCACTTGA | /5Phos/GTCATCAAGTGGAATCAAGTGGAA |
| TTCCGAGAGCTTCCGAGAGC | /5Phos/TTCGTTCCGAGAGCTTCCGAGAGC | /5Phos/GTCAGCTCTCGGAAGCTCTCGGAA |
| TTCCTGAGCCTTCCTGAGCC | /5Phos/TTCGTTCCTGAGCCTTCCTGAGCC | /5Phos/GTCAGGCTCAGGAAGGCTCAGGAA |
| TTCGAGTCGCTTCGAGTCGC | /5Phos/TTCGTTCGAGTCGCTTCGAGTCGC | /5Phos/GTCAGCGACTCGAAGCGACTCGAA |
| TTCGTGCCAGTTCGTGCCAG | /5Phos/TTCGTTCGTGCCAGTTCGTGCCAG | /5Phos/GTCACTGGCACGAACTGGCACGAA |
| TTCTACACCATTCTACACCA | /5Phos/TTCGTTCTACACCATTCTACACCA | /5Phos/GTCATGGTGTAGAATGGTGTAGAA |
| TTCTCGCTCCTTCTCGCTCC | /5Phos/TTCGTTCTCGCTCCTTCTCGCTCC | /5Phos/GTCAGGAGCGAGAAGGAGCGAGAA |
| TTCTGGCCACTTCTGGCCAC | /5Phos/TTCGTTCTGGCCACTTCTGGCCAC | /5Phos/GTCAGTGGCCAGAAGTGGCCAGAA |
| TTGAATGGTGTTGAATGGTG | /5Phos/TTCGTTGAATGGTGTTGAATGGTG | /5Phos/GTCACACCATTCAACACCATTCAA |
| TTGAGCCGACTTGAGCCGAC | /5Phos/TTCGTTGAGCCGACTTGAGCCGAC | /5Phos/GTCAGTCGGCTCAAGTCGGCTCAA |
| TTGCGCCAAGTTGCGCCAAG | /5Phos/TTCGTTGCGCCAAGTTGCGCCAAG | /5Phos/GTCACTTGGCGCAACTTGGCGCAA |
| TTGCTCTTAGTTGCTCTTAG | /5Phos/TTCGTTGCTCTTAGTTGCTCTTAG | /5Phos/GTCACTAAGAGCAACTAAGAGCAA |
| TTGGCAAGGCTTGGCAAGGC | /5Phos/TTCGTTGGCAAGGCTTGGCAAGGC | /5Phos/GTCAGCCTTGCCAAGCCTTGCCAA |
| TTGGCTCACGTTGGCTCACG | /5Phos/TTCGTTGGCTCACGTTGGCTCACG | /5Phos/GTCACGTGAGCCAACGTGAGCCAA |
| TTGGCTTCCATTGGCTTCCA | /5Phos/TTCGTTGGCTTCCATTGGCTTCCA | /5Phos/GTCATGGAAGCCAATGGAAGCCAA |
| TTGTGGATACTTGTGGATAC | /5Phos/TTCGTTGTGGATACTTGTGGATAC | /5Phos/GTCAGTATCCACAAGTATCCACAA |
| AACACGGATGAACACGGATG | /5Phos/TTCGAACACGGATGAACACGGATG | /5Phos/GTCACATCCGTGTTCATCCGTGTT |
| AACAGACCGGAACAGACCGG | /5Phos/TTCGAACAGACCGGAACAGACCGG | /5Phos/GTCACCGGTCTGTTCCGGTCTGTT |
| AACAGTGATCAACAGTGATC | /5Phos/TTCGAACAGTGATCAACAGTGATC | /5Phos/GTCAGATCACTGTTGATCACTGTT |
| AACCATCTTGAACCATCTTG | /5Phos/TTCGAACCATCTTGAACCATCTTG | /5Phos/GTCACAAGATGGTTCAAGATGGTT |
| AACGGTGACGAACGGTGACG | /5Phos/TTCGAACGGTGACGAACGGTGACG | /5Phos/GTCACGTCACCGTTCGTCACCGTT |
| AACTACGCGGAACTACGCGG | /5Phos/TTCGAACTACGCGGAACTACGCGG | /5Phos/GTCACCGCGTAGTTCCGCGTAGTT |
| AACTAGGCTTAACTAGGCTT | /5Phos/TTCGAACTAGGCTTAACTAGGCTT | /5Phos/GTCAAAGCCTAGTTAAGCCTAGTT |
| AACTGGCGTGAACTGGCGTG | /5Phos/TTCGAACTGGCGTGAACTGGCGTG | /5Phos/GTCACACGCCAGTTCACGCCAGTT |
| AAGACAGGATAAGACAGGAT | /5Phos/TTCGAAGACAGGATAAGACAGGAT | /5Phos/GTCAATCCTGTCTTATCCTGTCTT |
| AAGAGCCAGTAAGAGCCAGT | /5Phos/TTCGAAGAGCCAGTAAGAGCCAGT | /5Phos/GTCAACTGGCTCTTACTGGCTCTT |
| AAGAGTATCGAAGAGTATCG | /5Phos/TTCGAAGAGTATCGAAGAGTATCG | /5Phos/GTCACGATACTCTTCGATACTCTT |
| AAGGAACACTAAGGAACACT | /5Phos/TTCGAAGGAACACTAAGGAACACT | /5Phos/GTCAAGTGTTCCTTAGTGTTCCTT |
| AAGTCGCAGGAAGTCGCAGG | /5Phos/TTCGAAGTCGCAGGAAGTCGCAGG | /5Phos/GTCACCTGCGACTTCCTGCGACTT |
| AATCACAGTCAATCACAGTC | /5Phos/TTCGAATCACAGTCAATCACAGTC | /5Phos/GTCAGACTGTGATTGACTGTGATT |
| AATCGCCATTAATCGCCATT | /5Phos/TTCGAATCGCCATTAATCGCCATT | /5Phos/GTCAAATGGCGATTAATGGCGATT |
| AATTGCGGCCAATTGCGGCC | /5Phos/TTCGAATTGCGGCCAATTGCGGCC | /5Phos/GTCAGGCCGCAATTGGCCGCAATT |
| ACAACTCGCGACAACTCGCG | /5Phos/TTCGACAACTCGCGACAACTCGCG | /5Phos/GTCACGCGAGTTGTCGCGAGTTGT |
| ACAAGCTGCGACAAGCTGCG | /5Phos/TTCGACAAGCTGCGACAAGCTGCG | /5Phos/GTCACGCAGCTTGTCGCAGCTTGT |
| ACACCAATTCACACCAATTC | /5Phos/TTCGACACCAATTCACACCAATTC | /5Phos/GTCAGAATTGGTGTGAATTGGTGT |
| ACAGAATGGTACACGAAGGT | /5Phos/TTCGACACGAAGGTACACGAAGGT | /5Phos/GTCAACCTTCGTGTACCTTCGTGT |
| ACAGAATGTGACAGAATGTG | /5Phos/TTCGACAGAATGTGACAGAATGTG | /5Phos/GTCACACATTCTGTCACATTCTGT |
| ACATAGTGGTACATAGTGGT | /5Phos/TTCGACATAGTGGTACATAGTGGT | /5Phos/GTCAACCACTATGTACCACTATGT |
| ACCACCACGTACCACCACGT | /5Phos/TTCGACCACCACGTACCACCACGT | /5Phos/GTCAACGTGGTGGTACGTGGTGGT |
| ACCGACAGCTACCGACAGCT | /5Phos/TTCGACCGACAGCTACCGACAGCT | /5Phos/GTCAAGCTGTCGGTAGCTGTCGGT |
| ACCGGCTAGTACCGGCTAGT | /5Phos/TTCGACCGGCTAGTACCGGCTAGT | /5Phos/GTCAACTAGCCGGTACTAGCCGGT |
| ACCGTAGATTACCGTAGATT | /5Phos/TTCGACCGTAGATTACCGTAGATT | /5Phos/GTCAAATCTACGGTAATCTACGGT |
| ACCGTGACTTACCGTGACTT | /5Phos/TTCGACCGTGACTTACCGTGACTT | /5Phos/GTCAAAGTCACGGTAAGTCACGGT |
| ACCTCACAACACCTCACAAC | /5Phos/TTCGACCTCACAACACCTCACAAC | /5Phos/GTCAGTTGTGAGGTGTTGTGAGGT |
| ACCTTGTCCTACCTTGTCCT | /5Phos/TTCGACCTTGTCCTACCTTGTCCT | /5Phos/GTCAAGGACAAGGTAGGACAAGGT |
| ACGAAGCAGGACGAAGCAGG | /5Phos/TTCGACGAAGCAGGACGAAGCAGG | /5Phos/GTCACCTGCTTCGTCCTGCTTCGT |
| ACGACTGGACACGACTGGAC | /5Phos/TTCGACGACTGGACACGACTGGAC | /5Phos/GTCAGTCCAGTCGTGTCCAGTCGT |
| ACGAGAGCAGACGAGAGCAG | /5Phos/TTCGACGAGAGCAGACGAGAGCAG | /5Phos/GTCACTGCTCTCGTCTGCTCTCGT |
| ACGCCTGTTGACGCCTGTTG | /5Phos/TTCGACGCCTGTTGACGCCTGTTG | /5Phos/GTCACAACAGGCGTCAACAGGCGT |
| ACGCGACTTCACGCGACTTC | /5Phos/TTCGACGCGACTTCACGCGACTTC | /5Phos/GTCAGAAGTCGCGTGAAGTCGCGT |
| ACGGATTGATACGGATTGAT | /5Phos/TTCGACGGATTGATACGGATTGAT | /5Phos/GTCAATCAATCCGTATCAATCCGT |
| ACGGCTCATGACGGCTCATG | /5Phos/TTCGACGGCTCATGACGGCTCATG | /5Phos/GTCACATGAGCCGTCATGAGCCGT |
| ACGGTAAGATACGGTAAGAT | /5Phos/TTCGACGGTAAGATACGGTAAGAT | /5Phos/GTCAATCTTACCGTATCTTACCGT |
| ACGGTAGCACACGGTAGCAC | /5Phos/TTCGACGGTAGCACACGGTAGCAC | /5Phos/GTCAGTGCTACCGTGTGCTACCGT |
| ACGGTGTTCGACGGTGTTCG | /5Phos/TTCGACGGTGTTCGACGGTGTTCG | /5Phos/GTCACGAACACCGTCGAACACCGT |
| ACGTCGATGGACGTCGATGG | /5Phos/TTCGACGTCGATGGACGTCGATGG | /5Phos/GTCACCATCGACGTCCATCGACGT |
| ACGTGCAGACACGTGCAGAC | /5Phos/TTCGACGTGCAGACACGTGCAGAC | /5Phos/GTCAGTCTGCACGTGTCTGCACGT |
| ACGTTCCAGCACGTTCCAGC | /5Phos/TTCGACGTTCCAGCACGTTCCAGC | /5Phos/GTCAGCTGGAACGTGCTGGAACGT |
| ACTAGCTTGTACTAGCTTGT | /5Phos/TTCGACTAGCTTGTACTAGCTTGT | /5Phos/GTCAACAAGCTAGTACAAGCTAGT |
| ACTAGGACGCACTAGGACGC | /5Phos/TTCGACTAGGACGCACTAGGACGC | /5Phos/GTCAGCGTCCTAGTGCGTCCTAGT |
| ACTCACCTGGACTCACCTGG | /5Phos/TTCGACTCACCTGGACTCACCTGG | /5Phos/GTCACCAGGTGAGTCCAGGTGAGT |
| ACTCATATCGACTCATATCG | /5Phos/TTCGACTCATATCGACTCATATCG | /5Phos/GTCACGATATGAGTCGATATGAGT |
| ACTGACCGTGACTGACCGTG | /5Phos/TTCGACTGACCGTGACTGACCGTG | /5Phos/GTCACACGGTCAGTCACGGTCAGT |
| ACTTCTAACCACTTCTAACC | /5Phos/TTCGACTTCTAACCACTTCTAACC | /5Phos/GTCAGGTTAGAAGTGGTTAGAAGT |
| ACTTGGCGCTACTTGGCGCT | /5Phos/TGACACTTGGCGCTACTTGGCGCT | /5Phos/GGGAAGCGCCAAGTAGCGCCAAGT |
| AGAACGCTCCAGAACGCTCC | /5Phos/TGACAGAACGCTCCAGAACGCTCC | /5Phos/GGGAGGAGCGTTCTGGAGCGTTCT |
| AGAACTTAGGAGAACTTAGG | /5Phos/TGACAGAACTTAGGAGAACTTAGG | /5Phos/GGGACCTAAGTTCTCCTAAGTTCT |
| AGAAGCGCATAGAAGCGCAT | /5Phos/TGACAGAAGCGCATAGAAGCGCAT | /5Phos/GGGAATGCGCTTCTATGCGCTTCT |
| AGACAATAGCAGACAATAGC | /5Phos/TGACAGACAATAGCAGACAATAGC | /5Phos/GGGAGCTATTGTCTGCTATTGTCT |
| AGACCGAGACAGACCGAGAC | /5Phos/TGACAGACCGAGACAGACCGAGAC | /5Phos/GGGAGTCTCGGTCTGTCTCGGTCT |
| AGACGCTGTCAGACGCTGTC | /5Phos/TGACAGACGCTGTCAGACGCTGTC | /5Phos/GGGAGACAGCGTCTGACAGCGTCT |
| AGACGTAGCGAGACGTAGCG | /5Phos/TGACAGACGTAGCGAGACGTAGCG | /5Phos/GGGACGCTACGTCTCGCTACGTCT |
| AGAGAGCTCTAGAGAGCTCT | /5Phos/TGACAGAGAGCTCTAGAGAGCTCT | /5Phos/GGGAAGAGCTCTCTAGAGCTCTCT |
| AGAGGCCACTAGAGGCCACT | /5Phos/TGACAGAGGCCACTAGAGGCCACT | /5Phos/GGGAAGTGGCCTCTAGTGGCCTCT |
| AGATTGCCGCAGATTGCCGC | /5Phos/TGACAGATTGCCGCAGATTGCCGC | /5Phos/GGGAGCGGCAATCTGCGGCAATCT |
| AGCACGATGCAGCACGATGC | /5Phos/TGACAGCACGATGCAGCACGATGC | /5Phos/GGGAGCATCGTGCTGCATCGTGCT |
| AGCAGAGAATAGCAGAGAAT | /5Phos/TGACAGCAGAGAATAGCAGAGAAT | /5Phos/GGGAATTCTCTGCTATTCTCTGCT |
| AGCCACAAGGAGCCACAAGG | /5Phos/TGACAGCCACAAGGAGCCACAAGG | /5Phos/GGGACCTTGTGGCTCCTTGTGGCT |
| AGCCAGGAAGAGCCAGGAAG | /5Phos/TGACAGCCAGGAAGAGCCAGGAAG | /5Phos/GGGACTTCCTGGCTCTTCCTGGCT |
| AGCTCTGGAGAGCTCTGGAG | /5Phos/TGACAGCTCTGGAGAGCTCTGGAG | /5Phos/GGGACTCCAGAGCTCTCCAGAGCT |
| AGCTTGGCAGAGCTTGGCAG | /5Phos/TGACAGCTTGGCAGAGCTTGGCAG | /5Phos/GGGACTGCCAAGCTCTGCCAAGCT |
| AGGAATAACGAGGAATAACG | /5Phos/TGACAGGAATAACGAGGAATAACG | /5Phos/GGGACGTTATTCCTCGTTATTCCT |
| AGGAATTGACAGGAATTGAC | /5Phos/TGACAGGAATTGACAGGAATTGAC | /5Phos/GGGAGTCAATTCCTGTCAATTCCT |
| AGGAGGAATTAGGAGGAATT | /5Phos/TGACAGGAGGAATTAGGAGGAATT | /5Phos/GGGAAATTCCTCCTAATTCCTCCT |
| AGGATAGGCCAGGATAGGCC | /5Phos/TGACAGGATAGGCCAGGATAGGCC | /5Phos/GGGAGGCCTATCCTGGCCTATCCT |
| AGGTGGCCTTAGGTGGCCTT | /5Phos/TGACAGGTGGCCTTAGGTGGCCTT | /5Phos/GGGAAAGGCCACCTAAGGCCACCT |
| AGGTGTTGCGAGGTGTTGCG | /5Phos/TGACAGGTGTTGCGAGGTGTTGCG | /5Phos/GGGACGCAACACCTCGCAACACCT |
| AGGTTAGGTGAGGTTAGGTG | /5Phos/TGACAGGTTAGGTGAGGTTAGGTG | /5Phos/GGGACACCTAACCTCACCTAACCT |
| AGTCCGTCCTAGTCCGTCCT | /5Phos/TGACAGTCCGTCCTAGTCCGTCCT | /5Phos/GGGAAGGACGGACTAGGACGGACT |
| AGTCGATCCGAGTCGATCCG | /5Phos/TGACAGTCGATCCGAGTCGATCCG | /5Phos/GGGACGGATCGACTCGGATCGACT |
| AGTCGCTGCTAGTCGCTGCT | /5Phos/TGACAGTCGCTGCTAGTCGCTGCT | /5Phos/GGGAAGCAGCGACTAGCAGCGACT |
| AGTCGTCCTCAGTCGTCCTC | /5Phos/TGACAGTCGTCCTCAGTCGTCCTC | /5Phos/GGGAGAGGACGACTGAGGACGACT |
| AGTGTTCCGTAGTGTTCCGT | /5Phos/TGACAGTGTTCCGTAGTGTTCCGT | /5Phos/GGGAACGGAACACTACGGAACACT |
| AGTTGCTCATAGTTGCTCAT | /5Phos/TGACAGTTGCTCATAGTTGCTCAT | /5Phos/GGGAATGAGCAACTATGAGCAACT |
| ATAACGTGAGATAACGTGAG | /5Phos/TGACATAACGTGAGATAACGTGAG | /5Phos/GGGACTCACGTTATCTCACGTTAT |
| ATACGCAGGCATACGCAGGC | /5Phos/TGACATACGCAGGCATACGCAGGC | /5Phos/GGGAGCCTGCGTATGCCTGCGTAT |
| ATACTGATGCATACTGATGC | /5Phos/TGACATACTGATGCATACTGATGC | /5Phos/GGGAGCATCAGTATGCATCAGTAT |
| ATAGTTCGTCATAGTTCGTC | /5Phos/TGACATAGTTCGTCATAGTTCGTC | /5Phos/GGGAGACGAACTATGACGAACTAT |
| ATATCTTCGCATATCTTCGC | /5Phos/TGACATATCTTCGCATATCTTCGC | /5Phos/GGGAGCGAAGATATGCGAAGATAT |
| ATATGCCTTCATATGCCTTC | /5Phos/TGACATATGCCTTCATATGCCTTC | /5Phos/GGGAGAAGGCATATGAAGGCATAT |
| ATCAGATCACATCAGATCAC | /5Phos/TGACATCAGATCACATCAGATCAC | /5Phos/GGGAGTGATCTGATGTGATCTGAT |
| ATCCAATCTGATCCAATCTG | /5Phos/TGACATCCAATCTGATCCAATCTG | /5Phos/GGGACAGATTGGATCAGATTGGAT |
| ATCCACAGCGATCCACAGCG | /5Phos/TGACATCCACAGCGATCCACAGCG | /5Phos/GGGACGCTGTGGATCGCTGTGGAT |
| ATCCGGAACGATCCGGAACG | /5Phos/TGACATCCGGAACGATCCGGAACG | /5Phos/GGGACGTTCCGGATCGTTCCGGAT |
| ATCGGCTTCCATCGGCTTCC | /5Phos/TGACATCGGCTTCCATCGGCTTCC | /5Phos/GGGAGGAAGCCGATGGAAGCCGAT |
| ATCGTCGGAGATCGTCGGAG | /5Phos/TGACATCGTCGGAGATCGTCGGAG | /5Phos/GGGACTCCGACGATCTCCGACGAT |
| ATCTCTCACGATCTCTCACG | /5Phos/TGACATCTCTCACGATCTCTCACG | /5Phos/GGGACGTGAGAGATCGTGAGAGAT |
| ATGCACCTGCATGCACCTGC | /5Phos/TGACATGCACCTGCATGCACCTGC | /5Phos/GGGAGCAGGTGCATGCAGGTGCAT |
| ATGCAGTCGCATGCAGTCGC | /5Phos/TGACATGCAGTCGCATGCAGTCGC | /5Phos/GGGAGCGACTGCATGCGACTGCAT |
| ATGCCGTAGGATGCCGTAGG | /5Phos/TGACATGCCGTAGGATGCCGTAGG | /5Phos/GGGACCTACGGCATCCTACGGCAT |
| ATGCGCGATCATGCGCGATC | /5Phos/TGACATGCGCGATCATGCGCGATC | /5Phos/GGGAGATCGCGCATGATCGCGCAT |
| ATGTGGTGATATGTGGTGAT | /5Phos/TGACATGTGGTGATATGTGGTGAT | /5Phos/GGGAATCACCACATATCACCACAT |
| ATTACGAGCCATTACGAGCC | /5Phos/TGACATTACGAGCCATTACGAGCC | /5Phos/GGGAGGCTCGTAATGGCTCGTAAT |
| ATTCCACGGCATTCCACGGC | /5Phos/TGACATTCCACGGCATTCCACGGC | /5Phos/GGGAGCCGTGGAATGCCGTGGAAT |
| ATTCGGCGTCATTCGGCGTC | /5Phos/TGACATTCGGCGTCATTCGGCGTC | /5Phos/GGGAGACGCCGAATGACGCCGAAT |
| ATTGGAAGCCATTGGAAGCC | /5Phos/TGACATTGGAAGCCATTGGAAGCC | /5Phos/GGGAGGCTTCCAATGGCTTCCAAT |
| ATTGTCGGCCATTGTCGGCC | /5Phos/TGACATTGTCGGCCATTGTCGGCC | /5Phos/GGGAGGCCGACAATGGCCGACAAT |
| CAACCGCTTGCAACCGCTTG | /5Phos/TGACCAACCGCTTGCAACCGCTTG | /5Phos/GGGACAAGCGGTTGCAAGCGGTTG |
| CAACTGGTGGCAACTGGTGG | /5Phos/TGACCAACTGGTGGCAACTGGTGG | /5Phos/GGGACCACCAGTTGCCACCAGTTG |
| CAAGATGGTGCAAGATGGTG | /5Phos/TGACCAAGATGGTGCAAGATGGTG | /5Phos/GGGACACCATCTTGCACCATCTTG |
| CAAGATTCGACAAGATTCGA | /5Phos/TGACCAAGATTCGACAAGATTCGA | /5Phos/GGGATCGAATCTTGTCGAATCTTG |
| CAAGCACGAGCAAGCACGAG | /5Phos/TGACCAAGCACGAGCAAGCACGAG | /5Phos/GGGACTCGTGCTTGCTCGTGCTTG |
| CAAGCCTGTGCAAGCCTGTG | /5Phos/TGACCAAGCCTGTGCAAGCCTGTG | /5Phos/GGGACACAGGCTTGCACAGGCTTG |
| CAAGCTCACGCAAGCTCACG | /5Phos/TGACCAAGCTCACGCAAGCTCACG | /5Phos/GGGACGTGAGCTTGCGTGAGCTTG |
| CAAGGTTGCGCAAGGTTGCG | /5Phos/TGACCAAGGTTGCGCAAGGTTGCG | /5Phos/GGGACGCAACCTTGCGCAACCTTG |
| CAAGTCGACGCAAGTCGACG | /5Phos/TGACCAAGTCGACGCAAGTCGACG | /5Phos/GGGACGTCGACTTGCGTCGACTTG |
| CACCACGAAGCACCACGAAG | /5Phos/TGACCACCACGAAGCACCACGAAG | /5Phos/GGGACTTCGTGGTGCTTCGTGGTG |
| CACCGATATTCACCGATATT | /5Phos/TGACCACCGATATTCACCGATATT | /5Phos/GGGAATATCGGTGAATATCGGTG |
| CACCGTCGAACACCGTCGAA | /5Phos/TGACCACCGTCGAACACCGTCGAA | /5Phos/GGGATTCGACGGTGTTCGACGGTG |
| CACCGTGACACACCGTGACA | /5Phos/TGACCACCGTGACACACCGTGACA | /5Phos/GGGATGTCACGGTGTGTCACGGTG |
| CACCTGCTGACACCTGCTGA | /5Phos/TGACCACCTGCTGACACCTGCTGA | /5Phos/GGGATCAGCAGGTGTCAGCAGGTG |
| CACGCACATACACGCACATA | /5Phos/TGACCACGCACATACACGCACATA | /5Phos/GGGATATGTGCGTGTATGTGCGTG |
| CACGCTAAGGCACGCTAAGG | /5Phos/TGACCACGCTAAGGCACGCTAAGG | /5Phos/GGGACCTTAGCGTGCCTTAGCGTG |
| CACGTAATCTCACGTAATCT | /5Phos/TGACCACGTAATCTCACGTAATCT | /5Phos/GGGAAGATTACGTGAGATTACGTG |
| CACGTGGAGTCACGTGGAGT | /5Phos/TGACCACGTGGAGTCACGTGGAGT | /5Phos/GGGAACTCCACGTGACTCCACGTG |
| CACTCGAGAGCACTCGAGAG | /5Phos/TGACCACTCGAGAGCACTCGAGAG | /5Phos/GGGACTCTCGAGTGCTCTCGAGTG |
| CACTCTCTGACACTCTCTGA | /5Phos/TGACCACTCTCTGACACTCTCTGA | /5Phos/GGGATCAGAGAGTGTCAGAGAGTG |
| CACTCTGGCTCACTCTGGCT | /5Phos/TGACCACTCTGGCTCACTCTGGCT | /5Phos/GGGAAGCCAGAGTGAGCCAGAGTG |
| CACTGCCATGCACTGCCATG | /5Phos/TGACCACTGCCATGCACTGCCATG | /5Phos/GGGACATGGCAGTGCATGGCAGTG |
| CACTTGAACTCACTTGAACT | /5Phos/TGACCACTTGAACTCACTTGAACT | /5Phos/GGGAAGTTCAAGTGAGTTCAAGTG |
| CAGACCTGAGCAGACCTGAG | /5Phos/TGACCAGACCTGAGCAGACCTGAG | /5Phos/GGGACTCAGGTCTGCTCAGGTCTG |
| CAGCGAGCATCAGCGAGCAT | /5Phos/TGACCAGCGAGCATCAGCGAGCAT | /5Phos/GGGAATGCTCGCTGATGCTCGCTG |
| CAGGAAGAGGCAGGAAGAGG | /5Phos/TGACCAGGAAGAGGCAGGAAGAGG | /5Phos/GGGACCTCTTCCTGCCTCTTCCTG |
| CAGTCTCATACAGTCTCATA | /5Phos/TGACCAGTCTCATACAGTCTCATA | /5Phos/GGGATATGAGACTGTATGAGACTG |
| GACTTATCGACAGTTATCGA | /5Phos/TGACCAGTTATCGACAGTTATCGA | /5Phos/GGGATCGATAACTGTCGATAACTG |
| CATACACGCGCATACACGCG | /5Phos/TGACCATACACGCGCATACACGCG | /5Phos/GGGACGCGTGTATGCGCGTGTATG |
| CATACCGACGCATACCGACG | /5Phos/TGACCATACCGACGCATACCGACG | /5Phos/GGGACGTCGGTATGCGTCGGTATG |
| CATCAATGGTCATCAATGGT | /5Phos/TGACCATCAATGGTCATCAATGGT | /5Phos/GGGAACCATTGATGACCATTGATG |
| CATGACACCGCATGACACCG | /5Phos/TGACCATGACACCGCATGACACCG | /5Phos/GGGACGGTGTCATGCGGTGTCATG |
| CATGGTTCGGCATGGTTCGG | /5Phos/TGACCATGGTTCGGCATGGTTCGG | /5Phos/GGGACCGAACCATGCCGAACCATG |
| CATTGGAGCGCATTGGAGCG | /5Phos/TGACCATTGGAGCGCATTGGAGCG | /5Phos/GGGACGCTCCAATGCGCTCCAATG |
| CCAACGAGAGCCAACGAGAG | /5Phos/TGACCCAACGAGAGCCAACGAGAG | /5Phos/GGGACTCTCGTTGGCTCTCGTTGG |
| CCAAGACCAGCCAAGACCAG | /5Phos/TGACCCAAGACCAGCCAAGACCAG | /5Phos/GGGACTGGTCTTGGCTGGTCTTGG |
| CCAATCACGGCCAATCACGG | /5Phos/TGACCCAATCACGGCCAATCACGG | /5Phos/GGGACCGTGATTGGCCGTGATTGG |
| CCACCGTTGTCCACCGTTGT | /5Phos/TGACCCACCGTTGTCCACCGTTGT | /5Phos/GGGAACAACGGTGGACAACGGTGG |
| CCAGATCGGACCAGATCGGA | /5Phos/TGACCCAGATCGGACCAGATCGGA | /5Phos/GGGATCCGATCTGGTCCGATCTGG |
| CCGAAGTCAGCCGAAGTCAG | /5Phos/TGACCCGAAGTCAGCCGAAGTCAG | /5Phos/GGGACTGACTTCGGCTGACTTCGG |
| CCGCTGAAGTCCGCTGAAGT | /5Phos/TGACCCGCTGAAGTCCGCTGAAGT | /5Phos/GGGAACTTCAGCGGACTTCAGCGG |
| CCGGACCATACCGGACCATA | /5Phos/TGACCCGGACCATACCGGACCATA | /5Phos/GGGATATGGTCCGGTATGGTCCGG |
| CCGTTCTAGGCCGTTCTAGG | /5Phos/TGACCCGTTCTAGGCCGTTCTAGG | /5Phos/GGGACCTAGAACGGCCTAGAACGG |
| CCTAATGCGGCCTAATGCGG | /5Phos/TGACCCTAATGCGGCCTAATGCGG | /5Phos/GGGACCGCATTAGGCCGCATTAGG |
| CCTATGACGACCTATGACGA | /5Phos/TGACCCTATGACGACCTATGACGA | /5Phos/GGGATCGTCATAGGTCGTCATAGG |
| CCTCACCAGTCCTCACCAGT | /5Phos/TGACCCTCACCAGTCCTCACCAGT | /5Phos/GGGAACTGGTGAGGACTGGTGAGG |
| CCTGAAGACGCCTGAAGACG | /5Phos/TGACCCTGAAGACGCCTGAAGACG | /5Phos/GGGACGTCTTCAGGCGTCTTCAGG |
| CCTGACTCCTCCTGACTCCT | /5Phos/TGACCCTGACTCCTCCTGACTCCT | /5Phos/GGGAAGGAGTCAGGAGGAGTCAGG |
| CGAAGAGTGGCGAAGAGTGG | /5Phos/TGACCGAAGAGTGGCGAAGAGTGG | /5Phos/GGGACCACTCTTCGCCACTCTTCG |
| CGAAGGTGGTCGAAGGTGGT | /5Phos/TGACCGAAGGTGGTCGAAGGTGGT | /5Phos/GGGAACCACCTTCGACCACCTTCG |
| CGACTAGCAGCGACTAGCAG | /5Phos/TGACCGACTAGCAGCGACTAGCAG | /5Phos/GGGACTGCTAGTCGCTGCTAGTCG |
| CGACTCGAGACGACTCGAGA | /5Phos/TGACCGACTCGAGACGACTCGAGA | /5Phos/GGGATCTCGAGTCGTCTCGAGTCG |
| CGACTTACAACGACTTACAA | /5Phos/TGACCGACTTACAACGACTTACAA | /5Phos/GGGATTGTAAGTCGTTGTAAGTCG |
| CGAGGATTAACGAGGATTAA | /5Phos/TGACCGAGGATTAACGAGGATTAA | /5Phos/GGGATTAATCCTCGTTAATCCTCG |
| CGAGGCATGTCGAGGCATGT | /5Phos/TGACCGAGGCATGTCGAGGCATGT | /5Phos/GGGAACATGCCTCGACATGCCTCG |
| CGAGTCTGCTCGAGTCTGCT | /5Phos/TGACCGAGTCTGCTCGAGTCTGCT | /5Phos/GGGAAGCAGACTCGAGCAGACTCG |
| CGAGTGAGCACGAGTGAGCA | /5Phos/TGACCGAGTGAGCACGAGTGAGCA | /5Phos/GGGATGCTCACTCGTGCTCACTCG |
| CGATCGGAAGCGATCGGAAG | /5Phos/TGACCGATCGGAAGCGATCGGAAG | /5Phos/GGGACTTCCGATCGCTTCCGATCG |
| CGATCTACCGCGATCTACCG | /5Phos/TGACCGATCTACCGCGATCTACCG | /5Phos/GGGACGGTAGATCGCGGTAGATCG |
| CGCCAAGCTTCGCCAAGCTT | /5Phos/TGACCGCCAAGCTTCGCCAAGCTT | /5Phos/GGGAAAGCTTGGCGAAGCTTGGCG |
| CGCCAGAATTCGCCAGAATT | /5Phos/TGACCGCCAGAATTCGCCAGAATT | /5Phos/GGGAAATTCTGGCGAATTCTGGCG |
| CGCGAATGGACGCGAATGGA | /5Phos/TGACCGCGAATGGACGCGAATGGA | /5Phos/GGGATCCATTCGCGTCCATTCGCG |
| CGCTCATCCTCGCTCATCCT | /5Phos/TGACCGCTCATCCTCGCTCATCCT | /5Phos/GGGAAGGATGAGCGAGGATGAGCG |
| CGCTCGAATGCGCTCGAATG | /5Phos/TGACCGCTCGAATGCGCTCGAATG | /5Phos/GGGACATTCGAGCGCATTCGAGCG |
| CGCTTACTATCGCTTACTAT | /5Phos/TGACCGCTTACTATCGCTTACTAT | /5Phos/GGGAATAGTAAGCGATAGTAAGCG |
| CGCTTAGCGTCGCTTAGCGT | /5Phos/TGACCGCTTAGCGTCGCTTAGCGT | /5Phos/GGGAACGCTAAGCGACGCTAAGCG |
| CGGACTTAAGCGGACTTAAG | /5Phos/TGACCGGACTTAAGCGGACTTAAG | /5Phos/GGGACTTAAGTCCGCTTAAGTCCG |
| CGGCGAACAACGGCGAACAA | /5Phos/TGACCGGCGAACAACGGCGAACAA | /5Phos/GGGATTGTTCGCCGTTGTTCGCCG |
| CGGCTTGGAACGGCTTGGAA | /5Phos/TGACCGGCTTGGAACGGCTTGGAA | /5Phos/GGGATTCCAAGCCGTTCCAAGCCG |
| CGGTAGTGCTCGGTAGTGCT | /5Phos/TGACCGGTAGTGCTCGGTAGTGCT | /5Phos/GGGAAGCACTACCGAGCACTACCG |
| CGGTTACACACGGTTACACA | /5Phos/TGACCGGTTACACACGGTTACACA | /5Phos/GGGATGTGTAACCGTGTGTAACCG |
| CGTACCGTGTCGTACCGTGT | /5Phos/TGACCGTACCGTGTCGTACCGTGT | /5Phos/GGGAACACGGTACGACACGGTACG |
| CGTAGAGCCACGTAGAGCCA | /5Phos/TGACCGTAGAGCCACGTAGAGCCA | /5Phos/GGGATGGCTCTACGTGGCTCTACG |
| CGTAGGACTGCGTAGGACTG | /5Phos/TGACCGTAGGACTGCGTAGGACTG | /5Phos/GGGACAGTCCTACGCAGTCCTACG |
| CGTATCACAACGTATCACAA | /5Phos/TGACCGTATCACAACGTATCACAA | /5Phos/GGGATTGTGATACGTTGTGATACG |
| CGTGTTGCGACGTGTTGCGA | /5Phos/TGACCGTGTTGCGACGTGTTGCGA | /5Phos/GGGATCGCAACACGTCGCAACACG |
| CGTTGGTCCACGTTGGTCCA | /5Phos/TGACCGTTGGTCCACGTTGGTCCA | /5Phos/GGGATGGACCAACGTGGACCAACG |
| CTACAGCCGACTACAGCCGA | /5Phos/TGACCTACAGCCGACTACAGCCGA | /5Phos/GGGATCGGCTGTAGTCGGCTGTAG |
| CTACGCAAGGCTACGCAAGG | /5Phos/TGACCTACGCAAGGCTACGCAAGG | /5Phos/GGGACCTTGCGTAGCCTTGCGTAG |
| CTACGGTGTGCTACGGTGTG | /5Phos/TGACCTACGGTGTGCTACGGTGTG | /5Phos/GGGACACACCGTAGCACACCGTAG |
| CTACGTTCCTCTACGTTCCT | /5Phos/TGACCTACGTTCCTCTACGTTCCT | /5Phos/GGGAAGGAACGTAGAGGAACGTAG |
| CTAGAGGCAGCTAGAGGCAG | /5Phos/TGACCTAGAGGCAGCTAGAGGCAG | /5Phos/GGGACTGCCTCTAGCTGCCTCTAG |
| CTAGGTCCAGCTAGGTCCAG | /5Phos/TGACCTAGGTCCAGCTAGGTCCAG | /5Phos/GGGACTGGACCTAGCTGGACCTAG |
| CTAGGTCGCTCTAGGTCGCT | /5Phos/TGACCTAGGTCGCTCTAGGTCGCT | /5Phos/GGGAAGCGACCTAGAGCGACCTAG |
| CTATCGCCGTCTATCGCCGT | /5Phos/TGACCTATCGCCGTCTATCGCCGT | /5Phos/GGGAACGGCGATAGACGGCGATAG |
| CTATGGATCTCTATGGATCT | /5Phos/TGACCTATGGATCTCTATGGATCT | /5Phos/GGGAAGATCCATAGAGATCCATAG |
| CTCGCGAGTTCTCGCGAGTT | /5Phos/TGACCTCGCGAGTTCTCGCGAGTT | /5Phos/GGGAAACTCGCGAGAACTCGCGAG |
| CTCGTGGCAACTCGTGGCAA | /5Phos/TGACCTCGTGGCAACTCGTGGCAA | /5Phos/GGGATTGCCACGAGTTGCCACGAG |
| CTCTACAACTCTCTACAACT | /5Phos/TGACCTCTACAACTCTCTACAACT | /5Phos/GGGAAGTTGTAGAGAGTTGTAGAG |
| CTCTATATCGCTCTATATCG | /5Phos/TGACCTCTATATCGCTCTATATCG | /5Phos/GGGACGATATAGAGCGATATAGAG |
| CTCTCCTTCACTCTCCTTCA | /5Phos/TGACCTCTCCTTCACTCTCCTTCA | /5Phos/GGGATGAAGGAGAGTGAAGGAGAG |
| CTCTCTTGCGCTCTCTTGCG | /5Phos/TGACCTCTCTTGCGCTCTCTTGCG | /5Phos/GGGACGCAAGAGAGCGCAAGAGAG |
| CTCTGCGTTGCTCTGCGTTG | /5Phos/TGACCTCTGCGTTGCTCTGCGTTG | /5Phos/GGGACAACGCAGAGCAACGCAGAG |
| CTGAATCCAGCTGAATCCAG | /5Phos/TGACCTGAATCCAGCTGAATCCAG | /5Phos/GGGACTGGATTCAGCTGGATTCAG |
| CTGAGCTTGGCTGAGCTTGG | /5Phos/TGACCTGAGCTTGGCTGAGCTTGG | /5Phos/GGGACCAAGCTCAGCCAAGCTCAG |
| CTGGATCCGACTGGATCCGA | /5Phos/TGACCTGGATCCGACTGGATCCGA | /5Phos/GGGATCGGATCCAGTCGGATCCAG |
| CTGGTCTGATCTGGTCTGAT | /5Phos/TGACCTGGTCTGATCTGGTCTGAT | /5Phos/GGGAATCAGACCAGATCAGACCAG |
| CTGTCCACAGCTGTCCACAG | /5Phos/TGACCTGTCCACAGCTGTCCACAG | /5Phos/GGGACTGTGGACAGCTGTGGACAG |
| CTGTCCTCCTCTGTCCTCCT | /5Phos/TGACCTGTCCTCCTCTGTCCTCCT | /5Phos/GGGAAGGAGGACAGAGGAGGACAG |
| CTGTCGGATGCTGTCGGATG | /5Phos/TGACCTGTCGGATGCTGTCGGATG | /5Phos/GGGACATCCGACAGCATCCGACAG |
| CTTCATCTGACTTCATCTGA | /5Phos/TGACCTTCATCTGACTTCATCTGA | /5Phos/GGGATCAGATGAAGTCAGATGAAG |
| CTTCCTGCGTCTTCCTGCGT | /5Phos/TGACCTTCCTGCGTCTTCCTGCGT | /5Phos/GGGAACGCAGGAAGACGCAGGAAG |
| CTTCGGCTAGCTTCGGCTAG | /5Phos/TGACCTTCGGCTAGCTTCGGCTAG | /5Phos/GGGACTAGCCGAAGCTAGCCGAAG |
| CTTCTTATGGCTTCTTATGG | /5Phos/TGACCTTCTTATGGCTTCTTATGG | /5Phos/GGGACCATAAGAAGCCATAAGAAG |
| CTTCTTGGATCTTCTTGGAT | /5Phos/TGACCTTCTTGGATCTTCTTGGAT | /5Phos/GGGAATCCAAGAAGATCCAAGAAG |
| CTTGCGATGGCTTGCGATGG | /5Phos/TGACCTTGCGATGGCTTGCGATGG | /5Phos/GGGACCATCGCAAGCCATCGCAAG |
| GAACCTCAGCGAACCTCAGC | /5Phos/TGACGAACGGATTAGAACGGATTA | /5Phos/GGGAGCTGAGGTTCGCTGAGGTTC |
| GAACGGATTAGAACGGATTA | /5Phos/TGACGAACGGATTAGAACGGATTA | /5Phos/GGGATAATCCGTTCTAATCCGTTC |
| GAACGTCATTGAACGTCATT | /5Phos/TGACGAACGTCATTGAACGTCATT | /5Phos/GGGAAATGACGTTCAATGACGTTC |
| GAACTGATCCGAACTGATCC | /5Phos/TGACGAACTGATCCGAACTGATCC | /5Phos/GGGAGGATCAGTTCGGATCAGTTC |
| GACAGCAGTCGACAGCAGTC | /5Phos/TGACGACAGCAGTCGACAGCAGTC | /5Phos/GGGAGACTGCTGTCGACTGCTGTC |
| GACCGAATGTGACCGAATGT | /5Phos/TGACGACCGAATGTGACCGAATGT | /5Phos/GGGAACATTCGGTCACATTCGGTC |
| GACGCCATCAGACGCCATCA | /5Phos/TGACGACGCCATCAGACGCCATCA | /5Phos/GGGATGATGGCGTCTGATGGCGTC |
| GACGCGATACGACGCGATAC | /5Phos/TGACGACGCGATACGACGCGATAC | /5Phos/GGGAGTATCGCGTCGTATCGCGTC |
| GACGCTGTGAGACGCTGTGA | /5Phos/TGACGACGCTGTGAGACGCTGTGA | /5Phos/GGGATCACAGCGTCTCACAGCGTC |
| GACGGACCTTGACGGACCTT | /5Phos/TGACGACGGACCTTGACGGACCTT | /5Phos/GGGAAAGGTCCGTCAAGGTCCGTC |
| GACGGAGTCTGACGGAGTCT | /5Phos/TGACGACGGAGTCTGACGGAGTCT | /5Phos/GGGAAGACTCCGTCAGACTCCGTC |
| GAGCACAACCGAGCACAACC | /5Phos/TGACGAGCACAACCGAGCACAACC | /5Phos/GGGAGGTTGTGCTCGGTTGTGCTC |
| GAGGAAGACCGAGGAAGACC | /5Phos/TGACGAGGAAGACCGAGGAAGACC | /5Phos/GGGAGGTCTTCCTCGGTCTTCCTC |
| GAGGCACGATGAGGCACGAT | /5Phos/TGACGAGGCACGATGAGGCACGAT | /5Phos/GGGAATCGTGCCTCATCGTGCCTC |
| GAGGTGAAGCGAGGTGAAGC | /5Phos/TGACGAGGTGAAGCGAGGTGAAGC | /5Phos/GGGAGCTTCACCTCGCTTCACCTC |
| GAGTCACACCGAGTCACACC | /5Phos/TGACGAGTCACACCGAGTCACACC | /5Phos/GGGAGGTGTGACTCGGTGTGACTC |
| GAGTCCAGACGAGTCCAGAC | /5Phos/TGACGAGTCCAGAcGAGTCCAGAC | /5Phos/GGGAGTCTGGACTCGTCTGGACTC |
| GATAACCTGTGATAACCTGT | /5Phos/TGACGATAACCTGTGATAACCTGT | /5Phos/GGGAACAGGTTATCACAGGTTATC |
| GATCCAAGGCGATCCAAGGC | /5Phos/TGACGATCCAAGGCGATCCAAGGC | /5Phos/GGGAGCCTTGGATCGCCTTGGATC |
| GATCGAGCCAGATCGAGCCA | /5Phos/TGACGATCGAGCCAGATCGAGCCA | /5Phos/GGGATGGCTCGATCTGGCTCGATC |
| GATGGCAATCGATGGCAATC | /5Phos/TGACGATGGCAATCGATGGCAATC | /5Phos/GGGAGATTGCCATCGATTGCCATC |
| GATTACCAACGATTACCAAC | /5Phos/TGACGATTACCAACGATTACCAAC | /5Phos/GGGAGTTGGTAATCGTTGGTAATC |
| GATTCGTCTTGATTCGTCTT | /5Phos/TGACGATTCGTCTTGATTCGTCTT | /5Phos/GGGAAAGACGAATCAAGACGAATC |
| GATTGTTGGAGATTGTTGGA | /5Phos/TGACGATTGTTGGAGATTGTTGGA | /5Phos/GGGATCCAACAATCTCCAACAATC |
| GCAACACGGAGCAACACGGA | /5Phos/TGACGCAACACGGAGCAACACGGA | /5Phos/GGGATCCGTGTTGCTCCGTGTTGC |
| GCAATGGTACGCAATGGTAC | /5Phos/TGACGCAATGGTACGCCATGGTAC | /5Phos/GGGAGTACCATTGCGTACCATTGC |
| GCACCACATTGCACCACATT | /5Phos/TGACGCACCACATTGCACCACATT | /5Phos/GGGAAATGTGGTGCAATGTGGTGC |
| GCAGATTGGCGCAGATTGGC | /5Phos/TGACGCAGATTGGCGCAGATTGGC | /5Phos/GGGAGCCAATCTGCGCCAATCTGC |
| GCAGGATAGCGCAGGATAGC | /5Phos/TGACGCAGGATAGCGCAGGATAGC | /5Phos/GGGAGCTATCCTGCGCTATCCTGC |
| GCATTAATCCGCATTAATCC | /5Phos/TGACGCATTAATCCGCATTAATCC | /5Phos/GGGAGGATTAATGCGGATTAATGC |
| GCCACCATCTGCCACCATCT | /5Phos/TGACGCCACCATCTGCCACCATCT | /5Phos/GGGAAGATGGTGGCAGATGGTGGC |
| GCCACCATCTGCCACCATCT | /5Phos/TGACGCCAGATACCGCCAGATACC | /5Phos/GGGAGGTATCTGGCGGTATCTGGC |
| GCCATAGATAGCCATAGATA | /5Phos/TGACGCCATAGATAGCCATAGATA | /5Phos/GGGATATCTATGGCTATCTATGGC |
| GCCATTGGACGCCATTGGAC | /5Phos/TCCCGCCATTGGACGCCATTGGAC | /5Phos/GTTGGTCCAATGGCGTCCAATGGC |
| GCCGAATCCTGCCGAATCCT | /5Phos/TCCCGCCGAATCCTGCCGAATCCT | /5Phos/GTTGAGGATTCGGCAGGATTCGGC |
| GCCGTGTCTAGCCGTGTCTA | /5Phos/TCCCGCCGTGTCTAGCCGTGTCTA | /5Phos/GTTGTAGACACGGCTAGACACGGC |
| GCCTTATCTCGCCTTATCTC | /5Phos/TCCCGCCTTATCTCGCCTTATCTC | /5Phos/GTTGGAGATAAGGCGAGATAAGGC |
| GCCTTCGCTTGCCTTCGCTT | /5Phos/TCCCGCCTTCGCTTGCCTTCGCTT | /5Phos/GTTGAAGCGAAGGCAAGCGAAGGC |
| GCGAAGTGGTGCGAAGTGGT | /5Phos/TCCCGCGAAGTGGTGCGAAGTGGT | /5Phos/GTTGACCACTTCGCACCACTTCGC |
| GCGAATATCTGCGAATATCT | /5Phos/TCCCGCGAATATCTGCGAATATCT | /5Phos/GTTGAGATATTCGCAGATATTCGC |
| GCGCTGATACGCGCTGATAC | /5Phos/TCCCGCGCTGATACGCGCTGATAC | /5Phos/GTTGGTATCAGCGCGTATCAGCGC |
| GCGGACTTCTGCGGACTTCT | /5Phos/TCCCGCGGACTTCTGCGGACTTCT | /5Phos/GTTGAGAAGTCCGCAGAAGTCCGC |
| GCGGATAAGTGCGGATAAGT | /5Phos/TCCCGCGGATAAGTGCGGATAAGT | /5Phos/GTTGACTTATCCGCACTTATCCGC |
| GCGGTCCATTGCGGTCCATT | /5Phos/TCCCGCGGTCCATTGCGGTCCATT | /5Phos/GTTGAATGGACCGCAATGGACCGC |
| GCTCAGTTAAGCTCAGTTAA | /5Phos/TCCCGCTCAGTTAAGCTCAGTTAA | /5Phos/GTTGTTAACTGAGCTTAACTGAGC |
| GCTCATTCTAGCTCATTCTA | /5Phos/TCCCGCTCATTCTAGCTCATTCTA | /5Phos/GTTGTAGAATGAGCTAGAATGAGC |
| GCTCCTAAGCGCTCCTAAGC | /5Phos/TCCCGCTCCTAAGCGCTCCTAAGC | /5Phos/GTTGGCTTAGGAGCGCTTAGGAGC |
| GCTGGAAGGAGCTGGAAGGA | /5Phos/TCCCGCTGGAAGGAGCTGGAAGGA | /5Phos/GTTGTCCTTCCAGCTCCTTCCAGC |
| GGAACAATGTGGAACAATGT | /5Phos/TCCCGGAACAATGTGGAACAATGT | /5Phos/GTTGACATTGTTCCACATTGTTCC |
| GGAACATCTCGGAACATCTC | /5Phos/TCCCGGAACATCTCGGAACATCTC | /5Phos/GTTGGAGATGTTCCGAGATGTTCC |
| GGAACCAGTAGGAACCAGTA | /5Phos/TCCCGGAACCAGTAGGAACCAGTA | /5Phos/GTTGTACTGGTTCCTACTGGTTCC |
| GGAACGACTTGGAACGACTT | /5Phos/TCCCGGAACGACTTGGAACGACTT | /5Phos/GTTGAAGTCGTTCCAAGTCGTTCC |
| GGAAGATGATGGAAGATGAT | /5Phos/TCCCGGAAGATGATGGAAGATGAT | /5Phos/GTTGATCATCTTCCATCATCTTCC |
| GGAAGGAGACGGAAGGAGAC | /5Phos/TCCCGGAAGGAGACGGAAGGAGAC | /5Phos/GTTGGTCTCCTTCCGTCTCCTTCC |
| GGAAGTGGTAGGAAGTGGTA | /5Phos/TCCCGGAAGTGGTAGGAAGTGGTA | /5Phos/GTTGTACCACTTCCTACCACTTCC |
| GGAATCAGGTGGAATCAGGT | /5Phos/TCCCGGAATCAGGTGGAATCAGGT | /5Phos/GTTGACCTGATTCCACCTGATTCC |
| GGAATGTGTAGGAATGTGTA | /5Phos/TCCCGGAATGTGTAGGAATGTGTA | /5Phos/GTTGTACACATTCCTACACATTCC |
| GGAGATAGGAGGAGATAGGA | /5Phos/TCCCGGAGATAGGAGGAGATAGGA | /5Phos/GTTGTCCTATCTCCTCCTATCTCC |
| GGAGCAATCCGGAGCAATCC | /5Phos/TCCCGGAGCAATCCGGAGCAATCC | /5Phos/GTTGGGATTGCTCCGGATTGCTCC |
| GGAGGACATCGGAGGACATC | /5Phos/TCCCGGAGGACATCGGAGGACATC | /5Phos/GTTCCATGTCCTCCGATGTCCTCC |
| GGCAATAGCCGGCAATAGCC | /5Phos/TCCCGGCAATAGCCGGCAATAGCC | /5Phos/GTTGGGCTATTGCCGGCTATTGCC |
| GGCAGAAGGAGGCAGAAGGA | /5Phos/TCCCGGCAGAAGGAGGCAGAAGGA | /5Phos/GTTGTCCTTCTGCCTCCTTCTGCC |
| GGCAGGAATAGGCAGGAATA | /5Phos/TCCCGGCAGGAATAGGCAGGAATA | /5Phos/GTTGTATTCCTGCCTATTCCTGCC |
| GGCATACACCGGCATACACC | /5Phos/TCCCGGCATACACCGGCATACACC | /5Phos/GTTGGGTGTATGCCGGTGTATGCC |
| GGCCGTTGTAGGCCGTTGTA | /5Phos/TCCCGGCCGTTGTAGGCCGTTGTA | /5Phos/GTTGTACAACGGCCTACAACGGCC |
| GGCCTGCTTAGGCCTGCTTA | /5Phos/TCCCGGCCTGCTTAGGCCTGCTTA | /5Phos/GTTGTAAGCAGGCCTAAGCAGGCC |
| GGCGAGGTAAGGCGAGGTAA | /5Phos/TCCCGGCGAGGTAAGGCGAGGTAA | /5Phos/GTTGTTACCTCGCCTTACCTCGCC |
| GGCGTGACATGGCGTGACAT | /5Phos/TCCCGGCGTGACATGGCGTGACAT | /5Phos/GTTGATGTCACGCCATGTCACGCC |
| GGCTCCAAGAGGCTCCAAGA | /5Phos/TCCCGGCTCCAAGAGGCTCCAAGA | /5Phos/GTTGTCTTGGAGCCTCTTGGAGCC |
| GGCTGCTCAAGGCTGCTCAA | /5Phos/TCCCGGCTGCTCAAGGCTGCTCAA | /5Phos/GTTGTTGAGCAGCCTTGAGCAGCC |
| GGCTGTGCTTGGCTGTGCTT | /5Phos/TCCCGGCTGTGCTTGGCTGTGCTT | /5Phos/GTTGAAGCACAGCCAAGCACAGCC |
| GGCTTCTGTCGGCTTCTGTC | /5Phos/TCCCGGCTTCTGTCGGCTTCTGTC | /5Phos/GTTGGACAGAAGCCGACAGAAGCC |
| GGTATCGCTTGGTATCGCTT | /5Phos/TCCCGGTATCGCTTGGTATCGCTT | /5Phos/GTTGAAGCGATACCAAGCGATACC |
| GGTCCTTCCAGGTCCTTCCA | /5Phos/TCCCGGTCCTTCCAGGTCCTTCCA | /5Phos/GTTGTGGAAGGACCTGGAAGGACC |
| GTTCGGTTATGGTCGGTTAT | /5Phos/TCCCGGTCGGTTATGGTCGGTTAT | /5Phos/GTTGATAACCGACCATAACCGACC |
| GGTCTGACGAGGTCTGACGA | /5Phos/TCCCGGTCTGACGAGGTCTGACGA | /5Phos/GTTGTCGTCAGACCTCGTCAGACC |
| GGTGACCACTGGTGACCACT | /5Phos/TCCCGGTGACCACTGGTGACCACT | /5Phos/GTTGAGTGGTCACCAGTGGTCACC |
| GGTGAGTCCAGGTGAGTCCA | /5Phos/TCCCGGTGAGTCCAGGTGAGTCCA | /5Phos/GTTGTGGACTCACCTGGACTCACC |
| GGTGGCGAATGGTGGCGAAT | /5Phos/TCCCGGTGGCGAATGGTGGCGAAT | /5Phos/GTTGATTCGCCACCATTCGCCACC |
| GGTGTATATCGGTGTATATC | /5Phos/TCCCGGTGTATATCGGTGTATATC | /5Phos/GTTGGATATACACCGATATACACC |
| GGTTCCTTAAGGTTCCTTAA | /5Phos/TCCCGGTTCCTTAAGGTTCCTTAA | /5Phos/GTTGTTAAGGAACCTTAAGGAACC |
| GGTTCTACAAGGTTCTACAA | /5Phos/TCCCGGTTCTACAAGGTTCTACAA | /5Phos/GTTGTTGTAGAACCTTGTAGAACC |
| GGTTGGCGTTGGTTGGCGTT | /5Phos/TCCCGGTTGGCGTTGGTTGGCGTT | /5Phos/GTTGAACGCCAACCAACGCCAACC |
| GTAACACGCTGTAACACGCT | /5Phos/TCCCGTAACACGCTGTAACACGCT | /5Phos/GTTGAGCGTGTTACAGCGTGTTAC |
| GTAGAGCGACGTAGAGCGAC | /5Phos/TCCCGTAGAGCGACGTAGAGCGAC | /5Phos/GTTGGTCGCTCTACGTCGCTCTAC |
| GTAGCTGCTCGTAGCTGCTC | /5Phos/TCCCGTAGCTGCTCGTAGCTGCTC | /5Phos/GTTGGAGCAGCTACGAGCAGCTAC |
| GTAGGTGGATGTAGGTGGAT | /5Phos/TCCCGTAGGTGGATGTAGGTGGAT | /5Phos/GTTGATCCACCTACATCCACCTAC |
| GTAGTCAGCCGTAGTCAGCC | /5Phos/TCCCGTAGTCAGCCGTAGTCAGCC | /5Phos/GTTGGGCTGACTACGGCTGACTAC |
| GTCCTTCCACGTCCTTCCAC | /5Phos/TCCCGTCCTTCCACGTCCTTCCAC | /5Phos/GTTGGTGGAAGGACGTGGAAGGAC |
| GTCGAGCAGTGTCGAGCAGT | /5Phos/TCCCGTCGAGCAGTGTCGAGCAGT | /5Phos/GTTGACTGCTCGACACTGCTCGAC |
| GTCGCACAGAGTCGCACAGA | /5Phos/TCCCGTCGCACAGAGTCGCACAGA | /5Phos/GTTGTCTGTGCGACTCTGTGCGAC |
| GTCGCACTTCGTCGCACTTC | /5Phos/TCCCGTCGCACTTCGTCGCACTTC | /5Phos/GTTGGAAGTGCGACGAAGTGCGAC |
| GTCTGGTGGTGTCTGGTGGT | /5Phos/TCCCGTCTGGTGGTGTCTGGTGGT | /5Phos/GTTGACCACCAGACACCACCAGAC |
| GTGAACTGTTGTGAACTGTT | /5Phos/TCCCGTGAACTGTTGTGAACTGTT | /5Phos/GTTGAACAGTTCACAACAGTTCAC |
| GTGCCATCCTGTGCCATCCT | /5Phos/TCCCGTGCCATCCTGTGCCATCCT | /5Phos/GTTGAGGATGGCACAGGATGGCAC |
| GTGCGTGAACGTGCGTGAAC | /5Phos/TCCCGTGCGTGAACGTGCGTGAAC | /5Phos/GTTGGTTCACGCACGTTCACGCAC |
| GTGGAGATGTGTGGAGATGT | /5Phos/TCCCGTGGAGATGTGTGGAGATGT | /5Phos/GTTGACATCTCCACACATCTCCAC |
| GTGGTGCAACGTGGTGCAAC | /5Phos/TCCCGTGGTGCAACGTGGTGCAAC | /5Phos/GTTGGTTGCACCACGTTGCACCAC |
| GTGTCGTTAAGTGTCGTTAA | /5Phos/TCCCGTGTCGTTAAGTGTCGTTAA | /5Phos/GTTGTTAACGACACTTAACGACAC |
| GTGTCTCAATGTGTCTCAAT | /5Phos/TCCCGTGTCTCAATGTGTCTCAAT | /5Phos/GTTGATTGAGACACATTGAGACAC |
| GTGTGATGACGTGTGATGAC | /5Phos/TCCCGTGTGATGACGTGTGATGAC | /5Phos/GTTGGTCATCACACGTCATCACAC |
| GTTAGATCGCGTTAGATCGC | /5Phos/TCCCGTTAGATCGCGTTAGATCGC | /5Phos/GTTGGCGATCTAACGCGATCTAAC |
| TAAGCCGATATAAGCCGATA | /5Phos/TCCCTAAGCCGATATAAGCCGATA | /5Phos/GTTGTATCGGCTTATATCGGCTTA |
| TAAGGACACCTAAGGACACC | /5Phos/TCCCTAAGGACACCTAAGGACACC | /5Phos/GTTGGGTGTCCTTAGGTGTCCTTA |
| TAAGGCCAAGTAAGGCCAAG | /5Phos/TCCCTAAGGCCAAGTAAGGCCAAG | /5Phos/GTTGCTTGGCCTTACTTGGCCTTA |
| TAATAGCGAGTAATAGCGAG | /5Phos/TCCCTAATAGCGAGTAATAGCGAG | /5Phos/GTTGCTCGCTATTACTCGCTATTA |
| TAATGGCCGGTAATGGCCGG | /5Phos/TCCCTAATGGCCGGTAATGGCCGG | /5Phos/GTTGCCGGCCATTACCGGCCATTA |
| TACCATTGGATACCATTGGA | /5Phos/TCCCTACCATTGGATACCATTGGA | /5Phos/GTTGTCCAATGGTATCCAATGGTA |
| TACGACCACCTACGACCACC | /5Phos/TCCCTACGACCACCTACGACCACC | /5Phos/GTTGGGTGGTCGTAGGTGGTCGTA |
| TACGACCTTATACGACCTTA | /5Phos/TCCCTACGACCTTATACGACCTTA | /5Phos/GTTGTAAGGTCGTATAAGGTCGTA |
| TACGTGATTCTACGTGATTC | /5Phos/TCCCTACGTGATTCTACGTGATTC | /5Phos/GTTGGAATCACGTAGAATCACGTA |
| TACTAGTCAGTACTAGTCAG | /5Phos/TCCCTACTAGTCAGTACTAGTCAG | /5Phos/GTTGCTGACTAGTACTGACTAGTA |
| TACTGACAAGTACTGACAAG | /5Phos/TCCCTACTGACAAGTACTGACAAG | /5Phos/GTTGCTTGTCAGTACTTGTCAGTA |
| TACTGCTGGCTACTGCTGGC | /5Phos/TCCCTACTGCTGGCTACTGCTGGC | /5Phos/GTTGGCCAGCAGTAGCCAGCAGTA |
| TAGACCGTAATAGACCGTAA | /5Phos/TCCCTAGACCGTAATAGACCGTAA | /5Phos/GTTGTTACGGTCTATTACGGTCTA |
| TAGACGAAGATAGACGAAGA | /5Phos/TCCCTAGACGAAGATAGACGAAGA | /5Phos/GTTGTCTTCGTCTATCTTCGTCTA |
| TAGCCTAGCCTAGCCTAGCC | /5Phos/TCCCTAGCCTAGCCTAGCCTAGCC | /5Phos/GTTGGGCTAGGCTAGGCTAGGCTA |
| TAGCGAATTCTAGCGAATTC | /5Phos/TCCCTAGCGAATTCTAGCGAATTC | /5Phos/GTTGGAATTCGCTAGAATTCGCTA |
| TAGCTGCCACTAGCTGCCAC | /5Phos/TCCCTAGCTGCCACTAGCTGCCAC | /5Phos/GTTGGTGGCAGCTAGTGGCAGCTA |
| TAGGTAGGCATAGGTAGGCA | /5Phos/TCCCTAGGTAGGCATAGGTAGGCA | /5Phos/GTTGTGCCTACCTATGCCTACCTA |
| TAGTCGTTACTAGTCGTTAC | /5Phos/TCCCTAGTCGTTACTAGTCGTTAC | /5Phos/GTTGGTAACGACTAGTAACGACTA |
| TAGTGCGAAGTAGTGCGAAG | /5Phos/TCCCTAGTGCGAAGTAGTGCGAAG | /5Phos/GTTGCTTCGCACTACTTCGCACTA |
| TAGTGGACGCTAGTGGACGC | /5Phos/TCCCTAGTGGACGCTAGTGGACGC | /5Phos/GTTGGCGTCCACTAGCGTCCACTA |
| TATAACGGTGTATAACGGTG | /5Phos/TCCCTATAACGGTGTATAACGGTG | /5Phos/GTTGCACCGTTATACACCGTTATA |
| TATAGAACCGTATAGAACCG | /5Phos/TCCCTATAGAACCGTATAGAACCG | /5Phos/GTTGCGGTTCTATACGGTTCTATA |
| TATCCGAAGGTATCCGAAGG | /5Phos/TCCCTATCCGAAGGTATCCGAAGG | /5Phos/GTTGCCTTCGGATACCTTCGGATA |
| TATCGGAGCCTATCGGAGCC | /5Phos/TCCCTATCGGAGCCTATCGGAGCC | /5Phos/GTTGGGCTCCGATAGGCTCCGATA |
| TATCGGCCTGTATCGGCCTG | /5Phos/TCCCTATCGGCCTGTATCGGCCTG | /5Phos/GTTGCAGGCCGATACAGGCCGATA |
| TATCGTCGGCTATCGTCGGC | /5Phos/TCCCTATCGTCGGCTATCGTCGGC | /5Phos/GTTGGCCGACGATAGCCGACGATA |
| TATGCGCCACTATGCGCCAC | /5Phos/TCCCTATGCGCCACTATGCGCCAC | /5Phos/GTTGGTGGCGCATAGTGGCGCATA |
| TATGGCCGTCTATGGCCGTC | /5Phos/TCCCTATGGCCGTCTATGGCCGTC | /5Phos/GTTGGACGGCCATAGACGGCCATA |
| TATTCTTCCGTATTCTTCCG | /5Phos/TCCCTATTCTTCCGTATTCTTCCG | /5Phos/GTTGCGGAAGAATACGGAAGAATA |
| TCAAGCAACGTCAAGCAACG | /5Phos/TCCCTCAAGCAACGTCAAGCAACG | /5Phos/GTTGCGTTGCTTGACGTTGCTTGA |
| TCAAGCAGTCTCAAGCAGTC | /5Phos/TCCCTCAAGCAGTCTCAAGCAGTC | /5Phos/GTTGGACTGCTTGAGACTGCTTGA |
| TCAAGTCCGATCAAGTCCGA | /5Phos/TCCCTCAAGTCCGATCAAGTCCGA | /5Phos/GTTGTCGGACTTGATCGGACTTGA |
| TCAATCGAGATCAATCGAGA | /5Phos/TCCCTCAATCGAGATCAATCGAGA | /5Phos/GTTGTCTCGATTGATCTCGATTGA |
| TCAATGTCGATCAATGTCGA | /5Phos/TCCCTCAATGTCGATCAATGTCGA | /5Phos/GTTGTCGACATTGATCGACATTGA |
| TCACTGAGGCTCACTGAGGC | /5Phos/TCCCTCACTGAGGCTCACTGAGGC | /5Phos/TCCGGCCTCAGTGAGCCTCAGTGA |
| TCAGCGAGACTCAGCGAGAC | /5Phos/TCCCTCAGCGAGACTCAGCGAGAC | /5Phos/GTTGGTCTCGCTGAGTCTCGCTGA |
| TCAGGAGGAATCAGGAGGAA | /5Phos/TCCCTCAGGAGGAATCAGGAGGAA | /5Phos/GTTGTTCCTCCTGATTCCTCCTGA |
| TCAGGCACAGTCAGGCACAG | /5Phos/TCCCTCAGGCACAGTCAGGCACAG | /5Phos/GTTGCTGTGCCTGACTGTGCCTGA |
| TCAGGCTTCCTCAGGCTTCC | /5Phos/TCCCTCAGGCTTCCTCAGGCTTCC | /5Phos/GTTGGGAAGCCTGAGGAAGCCTGA |
| TCCACACTCGTCCACACTCG | /5Phos/TCCCTCCACACTCGTCCACACTCG | /5Phos/GTTGCGAGTGTGGACGAGTGTGGA |
| TCCACAGCGATCCACAGCGA | /5Phos/TCCCTCCACAGCGATCCAGCTGCA | /5Phos/GTTGTCGCTGTGGATCGCTGTGGA |
| TCCAGCTGCATCCAGCTGCA | /5Phos/TCCCTCCAGCTGCATCCAGCTGCA | /5Phos/GTTGTGCAGCTGGATGCAGCTGGA |
| TCCATAATCCTCCATAATCC | /5Phos/TCCCTCCATAATCCTCCATAATCC | /5Phos/GTTGGGATTATGGAGGATTATGGA |
| TCCGGACCAATCCGGACCAA | /5Phos/TCCCTCCGGACCAATCCGGACCAA | /5Phos/GTTGTTGGTCCGGATTGGTCCGGA |
| TCCGTAACGGTCCGTAACGG | /5Phos/TCCCTCCGTAACGGTCCGTAACGG | /5Phos/GTTGCCGTTACGGACCGTTACGGA |
| TCCGTAGGTCTCCGTAGGTC | /5Phos/TCCCTCCGTAGGTCTCCGTAGGTC | /5Phos/GTTGGACCTACGGAGACCTACGGA |
| TCCGTCCAAGTCCGTCCAAG | /5Phos/TCCCTCCGTCCAAGTCCGTCCAAG | /5Phos/GTTGCTTGGACGGACTTGGACGGA |
| TCCTGAACCGTCCTGAACCG | /5Phos/TCCCTCCTGAACCGTCCTGAACCG | /5Phos/GTTGCGGTTCAGGACGGTTCAGGA |
| TCCTGGCATGTCCTGGCATG | /5Phos/TCCCTCCTGGCATGTCCTGGCATG | /5Phos/GTTGCATGCCAGGACATGCCAGGA |
| TCGCGCTACATCGCGCTACA | /5Phos/TCCCTCGCGCTACATCGCGCTACA | /5Phos/GTTGTGTAGCGCGATGTAGCGCGA |
| TCGGTTACCATCGGTTACCA | /5Phos/TCCCTCGGTTACCATCGGTTACCA | /5Phos/GTTGTGGTAACCGATGGTAACCGA |
| TCGTCCGTCATCGTCCGTCA | /5Phos/TCCCTCGTCCGTCATCGTCCGTCA | /5Phos/GTTGTGACGGACGATGACGGACGA |
| TCGTCCTCAGTCGTCCTCAG | /5Phos/TCCCTCGTCCTCAGTCGTCCTCAG | /5Phos/GTTGCTGAGGACGACTGAGGACGA |
| TCTACCGCTCTCTACCGCTC | /5Phos/TCCCTCTACCGCTCTCTACCGCTC | /5Phos/GTTGGAGCGGTAGAGAGCGGTAGA |
| TCTAGCTCGGTCTAGCTCGG | /5Phos/TCCCTCTAGCTCGGTCTAGCTCGG | /5Phos/GTTGCCGAGCTAGACCGAGCTAGA |
| TCTCGGCTGATCTCGGCTGA | /5Phos/TCCCTCTCGGCTGATCTCGGCTGA | /5Phos/GTTGTCAGCCGAGATCAGCCGAGA |
| TCTCGGTCAGTCTCGGTCAG | /5Phos/TCCCTCTCGGTCAGTCTCGGTCAG | /5Phos/GTTGCTGACCGAGACTGACCGAGA |
| TCTCTAGATCTCTCTAGATC | /5Phos/TCCCTCTCTAGATCTCTCTAGATC | /5Phos/GTTGGATCTAGAGAGATCTAGAGA |
| TCTGACCGCATCTGACCGCA | /5Phos/TCCCTCTGACCGCATCTGACCGCA | /5Phos/GTTGTGCGGTCAGATGCGGTCAGA |
| TCTGGACAGATCTGGACAGA | /5Phos/TCCCTCTGGACAGATCTGGACAGA | /5Phos/GTTGTCTGTCCAGATCTGTCCAGA |
| TCTGGATAAGTCTGGATAAG | /5Phos/TCCCTCTGGATAAGTCTGGATAAG | /5Phos/GTTGCTTATCCAGACTTATCCAGA |
| TCTTACGGCCTCTTACGGCC | /5Phos/TCCCTCTTACGGCCTCTTACGGCC | /5Phos/GTTGGGCCGTAAGAGGCCGTAAGA |
| TCTTGCATACTCTTGCATAC | /5Phos/TCCCTCTTGCATACTCTTGCATAC | /5Phos/GTTGGTATGCAAGAGTATGCAAGA |
| TGAACTTGGATGAACTTGGA | /5Phos/TCCCTGAACTTGGATGAACTTGGA | /5Phos/GTTGTCCAAGTTCATCCAAGTTCA |
| TGACACAGCGTGACACAGCG | /5Phos/TCCCTGACACAGCGTGACACAGCG | /5Phos/GTTGCGCTGTGTCACGCTGTGTCA |
| TGACAGGTCCTGACAGGTCC | /5Phos/TCCCTGACAGGTCCTGACAGGTCC | /5Phos/GTTGGGACCTGTCAGGACCTGTCA |
| TGACCTTCCGTGACCTTCCG | /5Phos/TCCCTGACCTTCCGTGACCTTCCG | /5Phos/GTTGCGGAAGGTCACGGAAGGTCA |
| TGACGTCGGATGACGTCGGA | /5Phos/TCCCTGACGTCGGATGACGTCGGA | /5Phos/GTTGTCCGACGTCATCCGACGTCA |
| TGACTATCTCTGACTATCTC | /5Phos/TCCCTGACTATCTCTGACTATCTC | /5Phos/GTTGGAGATAGTCAGAGATAGTCA |
| TGACTCCAGGTGACTCCAGG | /5Phos/TCCCTGACTCCAGGTGACTCCAGG | /5Phos/GTTGCCTGGAGTCACCTGGAGTCA |
| TGAGAGCAGGTGAGAGCAGG | /5Phos/TCCCTGAGAGCAGGTGAGAGCAGG | /5Phos/GTTGCCTGCTCTCACCTGCTCTCA |
| TGAGCCTCCATGAGCCTCCA | /5Phos/TCCCTGAGCCTCCATGAGCCTCCA | /5Phos/GTTGTGGAGGCTCATGGAGGCTCA |
| TGAGTATGGATGAGTATGGA | /5Phos/TCCCTGAGTATGGATGAGTATGGA | /5Phos/GTTGTCCATACTCATCCATACTCA |
| TGCAACATACTGCAACATAC | /5Phos/TCCCTGCAACATACTGCAACATAC | /5Phos/GTTGGTATGTTGCAGTATGTTGCA |
| TGCGCTGTAGTGCGCTGTAG | /5Phos/TCCCTGCGCTGTAGTGCGCTGTAG | /5Phos/GTTGCTACAGCGCACTACAGCGCA |
| TGCTAACCGGTGCTAACCGG | /5Phos/TCCCTGCTAACCGGTGCTAACCGG | /5Phos/GTTGCCGGTTAGCACCGGTTAGCA |
| TGCTCCACTGTGCTCCACTG | /5Phos/TCCCTGCTCCACTGTGCTCCACTG | /5Phos/GTTGCAGTGGAGCACAGTGGAGCA |
| TGGAAGGAGCTGGAAGGAGC | /5Phos/TCCCTGGAAGGAGCTGGAAGGAGC | /5Phos/GTTGGCTCCTTCCAGCTCCTTCCA |
| TGGCAGTGACTGGCAGTGAC | /5Phos/TCCCTGGCAGTGACTGGCAGTGAC | /5Phos/GTTGGTCACTGCCAGTCACTGCCA |
| TGGCATCAGCTGGCATCAGC | /5Phos/TCCCTGGCATCAGCTGGCATCAGC | /5Phos/GTTGGCTGATGCCAGCTGATGCCA |
| TGGCCTTATATGGCCTTATA | /5Phos/TCCCTGGCCTTATATGGCCTTATA | /5Phos/GTTGTATAAGGCCATATAAGGCCA |
| TGGCGAAGCATGGCGAAGCA | /5Phos/TCCCTGGCGAAGCATGGCGAAGCA | /5Phos/GTTGTGCTTCGCCATGCTTCGCCA |
| TGGCTTAAGATGGCTTAAGA | /5Phos/TCCCTGGCTTAAGATGGCTTAAGA | /5Phos/GTTGTCTTAAGCCATCTTAAGCCA |
| TGGTCAGCTCTGGTCAGCTC | /5Phos/TCCCTGGTCAGCTCTGGTCAGCTC | /5Phos/GTTGGAGCTGACCAGAGCTGACCA |
| TGGTTCCAACTGGTTCCAAC | /5Phos/TCCCTGGTTCCAACTGGTTCCAAC | /5Phos/GTTGGTTGGAACCAGTTGGAACCA |
| TGGTTGGTAATGGTTGGTAA | /5Phos/TCCCTGGTTGGTAATGGTTGGTAA | /5Phos/GTTGTTACCAACCATTACCAACCA |
| TGTACGCGCATGTACGCGCA | /5Phos/TCCCTGTACGCGCATGTACGCGCA | /5Phos/GTTGTGCGCGTACATGCGCGTACA |
| TGTACGCTGGTGTACGCTGG | /5Phos/TCCCTGTACGCTGGTGTACGCTGG | /5Phos/GTTGCCAGCGTACACCAGCGTACA |
| TGTAGCATTGTGTAGCATTG | /5Phos/TCCCTGTAGCATTGTGTAGCATTG | /5Phos/GTTGCAATGCTACACAATGCTACA |
| TGTAGTGCCGTGTAGTGCCG | /5Phos/TCCCTGTAGTGCCGTGTAGTGCCG | /5Phos/GTTGCGGCACTACACGGCACTACA |
| TGTGAATCTGTGTGAATCTG | /5Phos/TCCCTGTGAATCTGTGTGAATCTG | /5Phos/GTTGCAGATTCACACAGATTCACA |
| TGTTCCGTGGTGTTCCGTGG | /5Phos/TCCCTGTTCCGTGGTGTTCCGTGG | /5Phos/GTTGCCACGGAACACCACGGAACA |
| TTAGCGCGTGTTAGCGCGTG | /5Phos/TCCCTTAGCGCGTGTTAGCGCGTG | /5Phos/GTTGCACGCGCTAACACGCGCTAA |
| TTAGTTGGACTTAGTTGGAC | /5Phos/TCCCTTAGTTGGACTTAGTTGGAC | /5Phos/GTTGGTCCAACTAAGTCCAACTAA |
| TTCCAACTTCTTCCAACTTC | /5Phos/TCCCTTCCAACTTCTTCCAACTTC | /5Phos/GTTGGAAGTTGGAAGAAGTTGGAA |
| TTCGTCAGGTTCCGTCAGG | /5Phos/TCCCTTCCGTCAGGTTCCGTCAGG | /5Phos/GTTGCCTGACGGAACCTGACGGAA |
| TTCCTCACCGTTCCTCACCG | /5Phos/TCCCTTCCTCACCGTTCCTCACCG | /5Phos/GTTGCGGTGAGGAACGGTGAGGAA |
| TTCCTCCGACTTCCTCCGAC | /5Phos/TCCCTTCCTCCGACTTCCTCCGAC | /5Phos/GTTGGTCGGAGGAAGTCGGAGGAA |
| TTCGACCTGGTTCGACCTGG | /5Phos/TCCCTTCGACCTGGTTCGACCTGG | /5Phos/GTTGCCAGGTCGAACCAGGTCGAA |
| TTCGCGTGGATTCGCGTGGA | /5Phos/TCCCTTCGCGTGGATTCGCGTGGA | /5Phos/GTTGTCCACGCGAATCCACGCGAA |
| TTCGGAAGCGTTCGGAAGCG | /5Phos/TCCCTTCGGAAGCGTTCGGAAGCG | /5Phos/GTTGCGCTTCCGAACGCTTCCGAA |
| TTCTCGATTGTTCTCGATTG | /5Phos/TCCCTTCTCGATTGTTCTCGATTG | /5Phos/GTTGCAATCGAGAACAATCGAGAA |
| TTCTCTCTAGTTCTCTCTAG | /5Phos/TCCCTTCTCTCTAGTTCTCTCTAG | /5Phos/GTTGCTAGAGAGAACTAGAGAGAA |
| TTCTTGCGCGTTCTTGCGCG | /5Phos/TCCCTTCTTGCGCGTTCTTGCGCG | /5Phos/GTTGCGCGCAAGAACGCGCAAGAA |
| TTGAGGTCGGTTGAGGTCGG | /5Phos/TCCCTTGAGGTCGGTTGAGGTCGG | /5Phos/GTTGCCGACCTCAACCGACCTCAA |
| TTGCACACGCTTGCACACGC | /5Phos/TCCCTTGCACACGCTTGCACACGC | /5Phos/GTTGGCGTGTGCAAGCGTGTGCAA |
| TTGCGCTCTGTTGCGCTCTG | /5Phos/TCCCTTGCGCTCTGTTGCGCTCTG | /5Phos/GTTGCAGAGCGCAACAGAGCGCAA |
| TTGCTGCAGCTTGCTGCAGC | /5Phos/TCCCTTGCTGCAGCTTGCTGCAGC | /5Phos/GTTGGCTGCAGCAAGCTGCAGCAA |
| TTGGCGTCTGTTGGCGTCTG | /5Phos/TCCCTTGGCGTCTGTTGGCGTCTG | /5Phos/GTTGCAGACGCCAACAGACGCCAA |
| TTGGTCCTTCTTGGTCCTTC | /5Phos/TCCCTTGGTCCTTCTTGGTCCTTC | /5Phos/GTTGGAAGGACCAAGAAGGACCAA |
| TTGTCGAGAGTTGTCGAGAG | /5Phos/TCCCTTGTCGAGAGTTGTCGAGAG | /5Phos/GTTGCTCTCGACAACTCTCGACAA |
| TTGTCTGTACTTGTCTGTAC | /5Phos/TCCCTTGTCTGTACTTGTCTGTAC | /5Phos/GTTGGTACAGACAAGTACAGACAA |
| TTGTTCTCTCTTGTTCTCTC | /5Phos/TCCCTTGTTCTCTCTTGTTCTCTC | /5Phos/GTTGGAGAGAACAAGAGAGAACAA |
| AACACACGTTAACACACGTT | /5Phos/TCCCAACACACGTTAACACACGTT | /5Phos/GTTGAACGTGTGTTAACGTGTGTT |
| AACATCGAGTAACATCGAGT | /5Phos/TCCCAACATCGAGTAACATCGAGT | /5Phos/GTTGACTCGATGTTACTCGATGTT |
| AACCGAGGACAACCGAGGAC | /5Phos/TCCCAACCGAGGACAACCGAGGAC | /5Phos/GTTGGTCCTCGGTTGTCCTCGGTT |
| AACCTAGTAGAACCTAGTAG | /5Phos/TCCCAACCTAGTAGAACCTAGTAG | /5Phos/GTTGCTACTAGGTTCTACTAGGTT |
| AACGCATACTAACGCATACT | /5Phos/TCCCAACGCATACTAACGCATACT | /5Phos/GTTGAGTATGCGTTAGTATGCGTT |
| AACTAACCTCAACTAACCTC | /5Phos/TCCCAACTAACCTCAACTAACCTC | /5Phos/GTTGGAGGTTAGTTGAGGTTAGTT |
| AACTAAGGAGAACTAAGGAG | /5Phos/TCCCAACTAAGGAGAACTAAGGAG | /5Phos/GTTGCTCCTTAGTTCTCCTTAGTT |
| AACTCGTTCTAACTCGTTCT | /5Phos/TCCCAACTCGTTCTAACTCGTTCT | /5Phos/GTTGAGAACGAGTTAGAACGAGTT |
| AACTGCCGCTAACTGCCGCT | /5Phos/TCCCAACTGCCGCTAACTGCCGCT | /5Phos/GTTGAGCGGCAGTTAGCGGCAGTT |

Each unique label may comprise two or more detectable oligonucleotide tags. The two or more tags may be three or more tags, four or more tags, or five or more tags. In some embodiments, a unique label may comprise 2, 3, 4, 5, 6, 7, 8, 9, 10, 15, 20, 30, 40, 50, 100 or more detectable tags.

The tags are typically bound to each other, typically in a directional manner. Methods for sequentially attaching nucleic acids such as oligonucleotides to each other are known in the art and include, but are not limited to, ligation and polymerization, or a combination of both (see, e.g., Green and Sambrook. Molecular Cloning: A Laboratory Manual, Fourth Edition, 2012). Ligation reactions include blunt end ligation and cohesive overhang ligation. In some instances, ligation may comprise both blunt end and cohesive overhang ligation. A cohesive overhang is a single stranded end sequence (attached to a double stranded sequence) capable of binding to another single stranded sequence thereby forming a double stranded sequence. A cohesive overhang may be generated by a polymerase, a restriction endonuclease, a combination of a polymerase and a restriction endonuclease, or a Uracil-Specific Excision Reagent (USER™) enzyme (New England BioLabs Inc., Ipswich, Mass.) or a combination of a Uracil DNA glycosylase enzyme and a DNA glycosylase-lyase Exonuclease VIII enzyme. A cohesive overhang may be a thymidine tail. Polymerization reactions include enzyme-mediated polymerization such as a polymerase-mediated fill-reaction.

Methods for detecting and analyzing unique labels are known in the art. In some embodiments, detection may comprise determining the presence, number, and/or order of detectable tags that comprise a unique label. Methods of sequencing oligonucleotides and nucleic acids are well known in the art (see, e.g., WO93/23564, WO98/28440 and WO98/13523; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

### Generation of Unique Labels

In some aspects, the invention provides methods for generating unique labels. The methods typically use a plurality of detectable tags to generate unique labels. In some embodiments, a unique label is produced by sequentially attaching two or more detectable oligonucleotide tags to each other. The detectable tags may be present or provided in a plurality of detectable tags. The same or a different plurality of tags may be used as the source of each detectable tag comprised in a unique label. In other words, a plurality of tags may be subdivided into subsets and single subsets may be used as the source for each tag. This is exemplified in at least FIG. 1.

A plurality of tags may comprise 2, 3, 4, 5, 6, 7, 8, 9, 10, 10², 10³, 10⁴, 10⁵, or 10⁶, or more tags. Typically, the tags within a plurality are unique relative to each other.

The methods of the invention allow an end user to generate a unique label for a plurality of agents using a number of tags that are less (and in some instances far less) than the number of agents to be labeled. The number of tags may be up to or about 10-fold, 10²-fold, 10³-fold, or 10⁴-fold less than the number of agents. The number of agents to be labeled will depend on the particular application. The invention contemplates uniquely labeling at least 10³, 10⁴, 10⁵, 10⁶, 10⁷, 10⁸, 10⁹, or 10¹⁰ or more agents. In some embodiments, the agent may comprise a plurality of nucleic acids. In some embodiments, the plurality of nucleic acids may comprise at least 10³, 10⁴, 10⁵, 10⁶, 10⁷, 10⁸, 10⁹, or 10¹⁰ nucleic acids.

In certain methods of the invention, agents, detectable tags, and resultant unique labels are all present in a contained volume and are thus physically separate from other agents, detectable tags, and resultant unique labels. In some instances, the contained volume is on the order of picoliters, nanoliters, or microliters. The contained volume may be a droplet such as an emulsion droplet.

As discussed herein, in some instances, an agent is attached to the unique label (or label intermediate) directly or indirectly. In these instances, once the detectable tag is attached to either the agent or the label intermediate, the droplets are ruptured (or broken) and their contents are pooled (and effectively mixed together). The contents of the pool may be introduced, at limiting dilution, into another plurality of emulsion droplets each of which may comprise a single detectable oligonucleotide tag (and optionally multiple copies of the oligonucleotide tag). This results in droplets that each contain an agent or a label intermediate, together with a single detectable oligonucleotide tag, and optionally reagents and enzymes required for tag attachment. Once the tag is attached, the droplets are again ruptured, and the process is repeated until a sufficient number of unique labels is generated.

In some embodiments a subset of the plurality of agents is present in the same container during attachment of a detectable label. In some embodiments, the plurality of agents is separated such that each agent in the plurality is in a separate container, e.g., an emulsion droplet.

In some embodiments, the process of pooling and subsequently separating the plurality of agents is performed n number of times, wherein n is the number of times required to generate (m₁)(m₂)(m₃) . . . (mₙ) number of combinations of detectable oligonucleotide tags, wherein (m₁)(m₂)(m₃) . . . (mₙ) number of combinations of detectable oligonucleotide tags is greater than the number of the plurality of agents.

The invention provides a method which may comprise
(a) labeling two or more first subsets of agents with a detectable oligonucleotide tag to produce agents within a subset that are identically labeled relative to each other and uniquely labeled relative to agents in other subsets;
(b) combining two or more subsets of uniquely-labeled agents to form a pool of agents, wherein the pool may comprise two or more second subsets of agents that are distinct from the two or more first subsets of agents;
(c) identically labeling two or more second subsets of agents with a second detectable oligonucleotide tag to produce agents within a second subset that are uniquely labeled relative to agents in the same or different second subsets; and
(d) repeating steps (b) and (c) until a number of unique labels is generated that exceeds the number of starting agents, wherein each unique label may comprise at least two detectable oligonucleotide tags.

The invention provides another method which may comprise
(a) providing a pool of agents;
(b) separating the pool of agents into sub-pools of agents;
(c) labeling agents in each sub-pool of with one of m₁ unique detectable oligonucleotide tags thereby producing sub-pools of labeled agents, wherein agents in a sub-pool are identically labeled to each other;
(d) combining sub-pools of labeled agents to create a pool of labeled agents;
(e) separating the pool of labeled agents into second sub-pools of agents;
(f) repeating steps (c) to (e) n times to produce agents labeled with n unique detectable oligonucleotide tags, wherein the pool in (a) consists of a number of agents that is less than (m₁)(m₂)(m₃) . . . (mₙ).

The invention provides another method which may comprise
(a) providing a population of library droplets which may comprise agents, wherein each droplet may comprise an agent;
(b) fusing each individual library droplet with a single detectable oligonucleotide tag droplet from a plurality of m₁ detectable oligonucleotide tag droplets, each detectable oligonucleotide tag droplet which may comprise a plurality of identical detectable oligonucleotide tag;
(c) labeling the agent with the detectable oligonucleotide tag in a fused droplet;
(d) harvesting labeled agents from the fused droplets and generating another population of library droplets which may comprise labeled agents; and
(e) repeating steps (b) to (d) n times to produce agents labeled with a unique label which may comprise n detectable oligonucleotides tags, wherein the n detectable oligonucleotide tags generate an (m₁)(m₂)(m₃) . . . (mₙ) number of combinations that is greater than the number of starting agents.

The invention provides another method which may comprise
(a) providing a population of library droplets which may comprise agents, wherein each droplet may comprise more than one agent;
(b) fusing each individual library droplet with a single detectable oligonucleotide tag droplet from a plurality of m₁ detectable oligonucleotide tag droplets, each detectable oligonucleotide tag droplet which may comprise a plurality of identical detectable oligonucleotide tag;
(c) labeling the more than one agents with the detectable oligonucleotide tag in a fused droplet;
(d) harvesting labeled agents from the fused droplets and generating another population of library droplets which may comprise labeled agents, wherein each droplet may comprise more than one agent; and
(e) repeating steps (b) to (d) n times to produce agents labeled with a unique label which may comprise n detectable oligonucleotides tags, wherein the n detectable oligonucleotide tags generate an (m₁)(m₂)(m₃) . . . (mₙ) number of combinations that is greater than the number of starting agents and optionally wherein the agents within the same droplet are labeled identically.

### Emulsions

Methods for making emulsion droplets are known in the art (see, e.g., WO 2006/040551; WO 2006/040554; WO 2004/002627; WO 2004/091763; WO 2005/021151; WO 2006/096571; WO 2007/089541; WO 2007/081385 and WO 2008/063227; U.S. Pat. No. 7,708,949; U.S. Patent Publication No. 20120122714, 20110000560, 20100022414; John Leamon, Darren R Link, Michael Egholm & Jonathan M Rothberg. Overview: methods and applications for droplet compartmentalization of biology. Nature Methods. Vol. 3, No. 7, 2006; E. Brouzes, M. Medkova, N. Savenelli, D. Marran, M. Twardowski, J. B. Hutchison, J. M. Rothberg, D. R. Link, N. Perrimon, M. L. Samuels. Droplet microfluidic technology for single-cell high-throughput screening. PNAS, 106, 14195. 2009; J. C. Baret, O. J. Miller, V. Taly, M. Ryckelynck, A. El-Harrak, L. Frenz, C. Rick, M. L. Samuels, J. B. Hutchison, J. J. Agresti, D. R. Link, D. A. Weitz and A. D. Griffiths. Fluorescence-activated droplet sorting (FADS): Efficient microfluidic cell sorting based on enzymatic activity. Lab Chip, 9, 1850. 2009; M. M. Kiss, L. Ortoleva-Donnelly, N. R. Beer, J. Warner, C. G. Bailey, B. W. Colston, J. M. Rothberg, D. R. Link, and J. H. Leamon. High-throughput quantitative polymerase chain reaction in picoliter droplets. Anal Chem. 2008 Dec. 1; 80(23): 8975-8981; Edd et al. Controlled encapsulation of single-cells into monodisperse picolitre drops. Lab Chip. 8(8): 1262-1264, 2008; Anna S L, Bontoux N, Stone H A (2003) Formation of dispersions using “flow focusing” in microchannels. Appl Phys Lett 82:364-366; all of which are incorporated herein by reference in their entirety).

A “droplet” or “emulsion droplet”, as used herein, is an isolated portion of a first fluid that is completely surrounded by a second fluid. The first and second fluids are immiscible with each other. For example, the discontinuous phase can be an aqueous solution and the continuous phase can a hydrophobic fluid such as an oil or a fluorocarbon oil. This is termed a water in oil emulsion. Alternatively, the emulsion may be an oil in water emulsion. In that example, the first liquid, which is dispersed in globules, is referred to as the discontinuous phase, whereas the second liquid is referred to as the continuous phase or the dispersion medium. The continuous phase can be an aqueous solution and the discontinuous phase is a hydrophobic fluid, such as an oil (e.g., decane, tetradecane, or hexadecane). The droplets or globules of oil in an oil in water emulsion are also referred to herein as “micelles”, whereas globules of water in a water in oil emulsion may be referred to as “reverse micelles”. In some cases, the droplets may be spherical or substantially spherical; however, in other cases, the droplets may be non-spherical.

The terms “droplet library” or “droplet libraries” are also referred to herein as an “emulsion library” or “emulsion libraries.” Examples of droplet libraries are collections of droplets that have different contents, ranging from DNA, primers, etc. The droplets range in size from roughly 0.5 micron to 500 micron in diameter, which corresponds to about 1 pico liter to 1 nano liter. However, droplets can be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges.

Droplets can be generated by infusing aqueous samples which may comprise library elements, e.g., agents, detectable tags, or combinations thereof, at a perpendicular angle to opposing oil streams. Droplets can be contained within a microfluidic channel. Microfluidic channels and method for manufacturing microfluidic channels are known in the art (see, e.g., McDonald J C, et al. (2000) Fabrication of microfluidic systems in poly(dimethylsiloxane) Electrophoresis 21:27-40; Siegel A C, et al.

2006) Cofabrication of electromagnets and microfluidic systems in poly(dimethylsiloxane)13. Angew Chem-Ger Edit 118:7031-7036; Agresti et al. (2009). Ultrahigh-throughput screening in drop-based microfluidics for directed evolution. PNAS 107(9), 4004-4009; all of which are incorporated herein in their entirety).

Other examples of microfluidic devices and approaches for use herein are disclosed in an application filed on Sep. 21, 2012, entitled “Systems and Methods for Droplet Tagging”, incorporated by reference herein in its entirety.

Droplets can be optionally merged. Merging can be accomplished, e.g., by passing an electrical field through a microfluidic channel to merge charged droplets, or by addition of a chemical that breaks emulsions. (See K. Ahn, J. Agresti, H. Chong, M. Marquez and D. A. Weitz, Appl. Phys. Lett., 2006, 88, 264105 and D Link, E Grasland-Mongrain, A Duri, F Sarrazin, Z Cheng, G Cristobal, M Marquez, and DA Weitz. Angew. Chem. Int. Ed. 2006, 45, 2556-2560 as examples.)

Generation of unique labels may occur in part or entirely in emulsion droplets. The unique label is generated in an emulsion droplet or in a series of emulsion droplets. A library of uniquely-labeled agents is generated using an emulsion droplet or a series of emulsion droplets.

### Mate-Pair Analysis

Another aspect of the invention addresses a fundamental issue associated with the creation of mate-pair (long distance linkage) libraries. Mate-pair libraries are useful for extracting distance information from sequences and are most typically used in genomic assemblies, detection of splicing in transcripts, and detection of genomic rearrangements. Traditionally, mate-pair libraries require that DNA molecules be circularized in order to directly join the ends together (i.e., as a mate-pair). The efficiency of circularization decreases as jump length increases, thus increasingly specialized techniques are required in order to prepare jumps of varying sizes. The methods described herein offer a major advantage over current methodologies in that mate-pair analysis is achieved without relying on circularization and is independent of jump length, thus making it a universal mate-pair protocol potentially suitable across a range of sequencing technologies.

In some embodiments, reactions are performed in emulsion droplets at single molecule dilution resulting in significant reductions in reagent costs, cycle time and input material. As described herein, emulsion droplets are used to segregate individual DNA molecules so that the ends of each DNA molecule can either be physically re-joined via ligation or informatically associated via analysis of the unique label.

Accordingly, in one aspect methods are provided for performing mate-pair analysis.

In some embodiments, the method may comprise:
(a) providing a population of library droplets which may comprise nucleic acids, wherein each droplet may comprise a nucleic acids end-labeled on its 5′ and 3′ ends with oligonucleotide label, wherein the oligonucleotide label on the 5′ end (the 5′ oligonucleotide label) and the oligonucleotide on the 3′ end (the 3′ oligonucleotide label) comprise a nucleotide cohesive overhang, and wherein the nucleotide cohesive overhang on the 5′ oligonucleotide label is complementary to the nucleotide cohesive overhang on the 3′ oligonucleotide label;
(b) fusing each individual library droplet with a droplet which may comprise a DNA fragmenting enzyme, thereby producing a fused droplet;
(c) fragmenting the nucleic acid with the 5′ and 3′ oligonucleotide labels in the fused droplet, thereby producing a fused droplet which may comprise a nucleic acid fragment which may comprise the 5′ oligonucleotide label and a nucleic acid fragment which may comprise the 3′ oligonucleotide label; and
(d) ligating the 5′ oligonucleotide label and the 3′ oligonucleotide label nucleic acid, thereby ligating the nucleic acid fragment which may comprise the 5′ oligonucleotide label and the nucleic acid fragment which may comprise the 3′ oligonucleotide label, thereby producing a ligated nucleic acid.

In some embodiments, the 5′ oligonucleotide label and/or the 3′ oligonucleotide may comprise a biotin label. In some embodiments, the method further may comprise (e) sequencing the ligated nucleic acid. In some embodiments, the DNA fragmenting agent is Nextera.

In another embodiment, the method may comprise:
(a) providing a population of library droplets which may comprise nucleic acids, wherein each droplet may comprise a nucleic acid which may comprise an oligonucleotide adapter,
(b) melting the nucleic acid;
(c) fusing each individual library droplet which may comprise a melted nucleic acid with a single index droplet from a plurality of m₁ index droplets, each index droplet which may comprise a first unique single-stranded detectable oligonucleotide tag, wherein the first unique single-stranded detectable oligonucleotide tag may comprise a region complementary to the oligonucleotide adapter,
(d) annealing the first unique single-stranded detectable oligonucleotide tag to the nucleic acid and performing a fill-in reaction, thereby producing an end-labeled nucleic;
(e) harvesting end-labeled nucleic acids from the fused droplets and generating another population of library droplets, wherein each droplet may comprise an end-labeled nucleic acid;
(f) melting the end-labeled nucleic acid;
(g) fusing each individual library droplet which may comprise a melted end-labeled nucleic acid with a single index droplet from a plurality of m₂ index droplets, each index droplet which may comprise a second unique single-stranded detectable oligonucleotide tag, wherein the second unique single-stranded detectable oligonucleotide tag may comprise a region complementary to the first unique single-stranded detectable oligonucleotide tag;
(h) annealing the second unique single-stranded detectable oligonucleotide tag to the nucleic acid and performing a fill-in reaction, thereby producing an end-labeled nucleic acid;
(i) harvesting end-labeled nucleic acid molecules from the fused droplets and generating another population of library droplets which may comprise end-labeled nucleic acids; and
(j) repeating steps (f) to (i) n times to produce nucleic acids end-labeled with n detectable oligonucleotide tags, wherein the n detectable oligonucleotide tags generate an (m₁)(m₂)(m₃) . . . (mₙ) number of combinations that is greater than the number of starting nucleic acids.

In another embodiment, the method may comprise:
sequentially end-labeling nucleic acids in a plurality, at their 5′ and 3′ ends, with a random combination of n detectable oligonucleotide tags,
wherein each end-labeled nucleic acid is
   (a) identically labeled at its 5′ and 3′ ends, and
   (b) uniquely labeled relative to other nucleic acids in the plurality,
wherein each detectable oligonucleotide tags is randomly and independently selected from a number of detectable oligonucleotide tags that is less than the number of nucleic acids, and n is the number of oligonucleotides attached to an end of a nucleic acid.

In some embodiments, the further may comprise fragmenting end-labeled nucleic acids into at least a 5′ fragment which may comprise the 5′ end of the nucleic acid attached to the random combination of n detectable oligonucleotide tags and into a 3′ fragment which may comprise the 3′ end of the nucleic acid attached to the random combination of n detectable oligonucleotide tags. In some embodiments, the 5′ and 3′ fragments are about 10-1000 bases (base pairs) in length, or about 10-500 bases in length, or about 10-200 bases in length. In some embodiments, the method further may comprise sequencing the 5′ and 3′ fragments.

In another embodiment, the method may comprise:
sequencing a pair of genomic nucleic acid fragments, wherein the genomic nucleic acid fragments are attached to identical unique labels at one of their ends that indicates the genomic nucleic acid fragments were separated by a known distance in a genome prior to fragmentation.

In some embodiments, the pair of nucleic acid fragments were separated by greater than 10 kb in the genome prior to fragmentation. In another embodiment, the pair of nucleic acid fragments were separated by greater than 40 kb in the genome prior to fragmentation. In some embodiments, the method further may comprise generating the pair of genomic nucleic acid fragments by fragmenting nucleic acids which may comprise genomic sequence and identical non-genomic sequence at their 5′ and 3′ ends.

In another aspect, the invention provides compositions. In one embodiment, the composition may comprise:
a plurality of paired nucleic acid fragments attached to unique labels at one end,
wherein paired nucleic acid fragments:
   (a) share an identical unique label at one end that is unique in the plurality, and
   (b) were separated from each other in a genome by a known distance prior to fragmentation.

In some embodiments, paired nucleic acid fragments were separated by greater than 10 kb in the genome prior to fragmentation. In another embodiment, the paired nucleic acid fragments were separated by greater than 40 kb in the genome prior to fragmentation. In some embodiments, the composition is produced using any of the methods described herein.

Examples of nucleic acids include, but are not limited to, genomic DNA, cDNA, PCR products, mRNA, total RNA, plasmids, or fragments thereof. In some embodiments, the nucleic acids are genomic DNA, cDNA, PCR products, or fragments thereof. Nucleic acids can be fragmented using methods described herein.

In some embodiments, the method further may comprise fragmenting uniquely end-labeled nucleic acids. Fragmenting of nucleic acids can be accomplished by methods described herein and those well-known in the art.

In one embodiment, the method may comprise sequencing a pair of genomic nucleic acid fragments, wherein the genomic nucleic acid fragments are attached to identical unique labels at one of their ends that indicates the genomic nucleic acid fragments were separated by a known distance in a genome prior to fragmentation. In some embodiments, the known distance is greater than 5, 10, 15, 20, 30, 40, 50, 100 kb or greater separation. Genomic nucleic acid fragments can come from any organismal genomic DNA, for example, human, mammalian, bacterial, fungal or plant genomic DNA. Genomic nucleic acid fragments can be generated by fragmentation methods known in the art (see, e.g., Green and Sambrook. Molecular Cloning: A Laboratory Manual, Fourth Edition, 2012). Examples of fragmentation include, but are not limited to, enzymatic (such as a nuclease), chemical (such as a DNA nicking agent) or mechanical (such as sonication) fragmentation. Fragmentation can be random, e.g., sequence and size unspecific, or ordered, e.g., sequence dependent and/or size-restricted. The fragments generated following label addition can be tailored to the limitations of the desired detection technology. For example, the fragments can be hundreds, thousands, millions or potentially billions of base pairs in length depending on the technology used to sequence the DNA.

### EXAMPLES

The following Examples are meant for illustrative purposes, and are not meant to be exclusive or limiting.

### Example 1

### Polymerase-Mediated Bioinformatic Association of Nucleic Acid Ends for Mate-Pair Analysis

Generation of End-Labeled Genomic DNA Fragments

A method for bioinformatically associating the ends of genomic DNA is outlined in FIG. 2. Genomic DNA is fragmented and size selected to a known size using techniques known in the art (e.g., sonication, cavitation, point-sink or mechanical shearing, or a DNA fragmenting enzyme and size-exclusion columns or gel purification). The genomic DNA is then A-tailed and ligated to a biotinylated, T-tailed asymmetric oligonucleotide adapter using methods known in the art (see, e.g. Maniatis, Molecular Cloning). Klenow exo-enzyme is commonly used to add a single nucleotide to the 3′ termini of DNA fragments). The adapter is a partial duplex to allow for annealing of the single-stranded oligonucleotide indexes described below.

One or more index libraries (preferably 1-4 libraries) are created such that each library contains approximately >1000 unique single-stranded oligonucleotide indexes, thus approximately 2000-4000 unique indexes are used. Index libraries may be created in droplets using standard flow focusing techniques. For a given library, each droplet will contain many copies of one unique single-stranded index. Droplets may contain some or all of the key components of a polymerase fill-in reaction (e.g., MgCl₂, dNTP, and Polymerase). Each unique single-stranded oligonucleotide index contains 3 distinct regions: sequence complimentary to the adapter (Ad) or to a previously added index sequence (B or C), a unique index sequence (Idx), and a sequence used to “capture” the next index oligonucleotide index which contains one or more dUTP nucleotides (B′/C′).

Fragmented genomic DNA ligated to an adapter is diluted to a desired concentration to control the number of molecules per droplet (e.g., a single DNA molecule per droplet or more than a single DNA molecule per droplet) and merged with (see above references for droplet merging) the first index library (Library “A” in FIG. 2).

The Ad region of each unique single-stranded oligonucleotide index binds to the adapter on each end of the fragmented genomic DNA molecule. A polymerase-mediated fill-in reaction is performed in each droplet, creating the complement to the index and capture regions on the each unique single-stranded oligonucleotide index a, and thus generating unique double-stranded oligonucleotide indexes.

Emulsion droplets are then broken using various mechanical or chemical reagents depending on the oil/surfactant utilized in the emulsion, resulting in the combination and mixing of the DNA from each droplet. Mixed DNA is then treated with USER™ enzyme (Uracil-Specific Excision Reagent, New England BioLabs Inc., Ipswich, Mass.), causing the capture portion of the double-stranded oligonucleotide index to be digested due to the presence of one or more dUTP nucleotides. This digestion reveals the nascent strand, which is complementary to a sequence contained in the next library of indexes (Library “B” in FIG. 2).

The process of fragmented genomic DNA dilution, merging with a droplet library, polymerase fill-in, breaking the droplets, and treatment with USER™ enzyme is repeated for the desired number of cycles, each time adding one new unique oligonucleotide index sequence to both ends of the fragmented genomic DNA.

After the final index addition, the result is fragmented genomic DNA uniquely end-labeled on both the 5′ and 3′ end with a unique label made up of many oligonucleotide indexes. The uniquely end-labeled fragmented genomic is then fragmented and the ends are collected via streptavidin beads, which recognized the biotin label on the adapter. Fragments can be ligated to technology specific sequencing adapters (e.g., Illumina adapters) and sequenced. Ends are informatically paired by matching the unique label on one fragment of DNA with the same unique label on the other fragment of DNA (see FIG. 7).

This method of bioinformatics association can also be used with other types of nucleic acids, such as RNA, cDNA, or PCR-amplified DNA, or any other type of construct where such a labeling scheme is required.

### Example 2

### Ligation-Mediated Bioinformatic Association of Nucleic Acid Ends in Emulsions for Mate-Pair Analysis

Validation of Ligation in Emulsions

A 34 bp adapter was designed. The adapter was biotinylated and T-tailed to force directionality of ligation to A-tailed lambda genomic DNA. Ligation was performed in an tube or an emulsion using 50 ng of lambda DNA and 50 ng of adapter. Lambda DNA was used as it is unlikely to form circles. Droplets were created by standard techniques (e.g., flow focusing at a T-junction using a PDMS-based microfluidic chip). Channel 1 contained DNA in ligase buffer (500 microliters) and channel 2 contained Quick Ligase in ligase buffer (500 microliters). PCR primers were designed to amplify internally within the lambda DNA (ligation-independent) or to amplify a portion of the adapter and the 5′ or 3′ end of the lambda DNA (ligation-dependent). Negative controls were performed in tubes to ensure ligation was ligase-dependent.

FIG. 3 shows that ligation was achieved in both tubes and emulsion droplets. The forward primer for the adapter and the 5′ primer for the lambda DNA only amplified in the presence of ligase, indicating that the adapter and the 5′ end of the lambda DNA had ligated together in both tubes and emulsion droplets. The same result was achieved using the reverse primer for the adapter and the 3′ primer for the lambda DNA, indicating that the adapter and the 3′ end of the lambda DNA had ligated together in both tubes and emulsion droplets. These results demonstrate that ligation can be successfully performed in emulsion droplets.

Generation of End-Labeled Genomic DNA Fragments

A method for bioinformatically associating the ends of genomic DNA is outlined in FIG. 7. Genomic DNA is fragmented and size selected to a known size using techniques known in the art as described in Example 1. The genomic DNA is then A-tailed and ligated to a biotinylated, T-tailed asymmetric oligonucleotide adapter using methods well known in the art as described in Example 1.

Multiple droplet libraries (preferably 2-4 libraries) are created such that each library contains approximately 1000 unique double-stranded oligonucleotide indexes, thus approximately 2000-4000 unique indexes are used. For a given library, each droplet will contain many copies of one unique double-stranded index. Droplets may contain some or all of the key components of a ligation reaction (e.g., MgCl₂, ATP, Ligase).

Fragmented genomic DNA ligated to an adapter is diluted to a desired concentration to control the number of molecules per droplet (e.g., a single DNA molecule per droplet or more than a single DNA molecule per droplet) and merged with the first index droplet library (Droplet Library “A” in FIG. 4).

A ligation reaction is performed in each droplet, joining each unique double-stranded oligonucleotide index to the adapter on each end of the genomic DNA. The emulsion is then broken and the DNA is phosphorylated so that a second index can be ligated to the end of the first index.

The process of fragmented genomic DNA dilution, merging with a droplet library (e.g. Droplet Library “B” or “C” in FIG. 4), ligation, breaking the droplets, and phosphorylation is repeated for the desired number of cycles, each time adding one new unique oligonucleotide index sequence to both ends of the fragmented genomic DNA.

After the final index addition, the result is fragmented genomic DNA uniquely end-labeled on both the 5′ and 3′ end with a unique label made up of many oligonucleotide indexes. The uniquely end-labeled fragmented genomic is then further fragmented and the ends are collected via streptavidin beads, which recognized the biotin label on the adapter. Fragments can be ligated to technology specific sequencing adapters (e.g., Illumina adapters) and sequenced. Ends are informatically paired by matching the unique label on one fragment of DNA with the same unique label on the other fragment of DNA as described in Example 1.

As with Example 1, this method can be used for other types of nucleic acids, such as RNA, cDNA, or PCR-amplified DNA, or any other type of construct where such a labeling scheme is required

Validation of Ligation-Mediated End-Labeling

Three libraries were created “in bulk” in microcentrifuge tubes from fragmented, end-repaired, A-tailed E. coli genomic DNA. For all three libraries, an initial ligation reaction was performed to add on a generic adapter to the ends of the E. coli genomic DNA. Genomic DNA libraries were then subjected to 1 (Library 1). 2 (Library 2), or 3 (Library 3) rounds of index ligation. Index ligation was performed by joining unique double-stranded oligonucleotide indexes to the adapter on each end of the genomic DNA. If required, the DNA was phosphorylated so that a second index could ligated to the end of the first index (two rounds of index ligation) or a third index could be ligated to the end of a second index (three rounds of index ligation). For round 1 and round 3 of index ligation, the same library/pool of indexes was used (pool A). For round 2, a library/pool of different indexes was used (pool B). As a final step, Illumina indexed adapters were ligated to all three genomic DNA libraries. Libraries were then pooled and sequence on an Illumina MiSeq (Illumina, San Diego, Calif.) using standard Illumina sequencing primers. Paired reads were identified and analyzed en masse (i.e. data from read 1 (3′ end read) and read 2 (5′ end read) was analyzed together as a single population). Sequencing data was analyzed by breaking up the reads into four separate, linear 8-mer populations (i.e. positions 1 through 4 in the read), since the indexes were each 8 bp in length. For each position, the number of reads containing index or adapter were measured.

FIGS. 13 and 14 depict the results of the total read population analysis (en masse analysis) of the index ligation method. Library 1, which underwent 1 round of index ligation, had an expected outcome of an index read in position 1 and an adapter read in position 2. Library 2, which underwent 2 rounds of index ligation, had an expected outcome of an index read in position 1 and 2 and an adapter read in position 3. Library 3, which underwent 3 rounds of index ligation, had an expected outcome of an index read in position 1 to 3 and an adapter read in position 4.

FIG. 15 depicts the results of read pair analysis of individual molecules that underwent the index ligation method. Instead of analyzing the data from read 1 (3′ end read) and read 2 (5′ end read) together, reads were paired so that a molecule-by-molecule analysis was performed. First, reads were paired based on their unique read identifier. Each read was then broken down into 4 positions (8-mers) per read as described above. For each library, the total number of read pairs and the total number of unique molecular outcomes were determined and are shown in FIG. 5 (Figure N). The composition of the top 10 most prevalent molecular outcomes and the number of pairs for each outcome are also shown in FIG. 5 (Figure N). It was determined that the most desired outcome (the correct expected outcome) occurred 6% of the time in Library 1, 4% of the time in Library 2, and 4% of the time in Library 3.

Thus, FIGS. 13-15 show that the expected outcome was achieved and thus index ligation was a valid method of generating a unique label.

### Example 3

### Fragment Amplification

To increase the number of read pairs properly mated via their unique index combination, three methods are proposed to maximize fragment end recovery. These methods utilize the fragment preparation and indexing techniques described above, but vary in their approach to recovering and amplifying fragment ends within the library construction process.

Transposome-Based Selection and Amplification of Ends

As shown in FIG. 22, DNA samples are sheared to a desired size then the “Cap” and random combinations of index sequences are symmetrically attached to the fragment ends via ligation. Following the final round of index ligation, a new adapter containing an Illumina sequencing primer (SP1) adjacent to the Illumina P7 sequence is attached to the ends of the molecules via ligation as described above. The population of molecules is then incubated in the presence of a transposome carrying a different Illumina sequencing primer (SP2) adjacent to the Illumina P5 sequence. This reaction creates many fragments where both ends are flanked by the Illumina P5 sequence, but only two fragments per molecule that carry both the Illumina P7 and P5 sequences. PCR amplification using primers to P5/P7 is performed in order to enrich/select the fragment ends.

Enrichment of Ends Via In Vitro Transcription

As seen in FIGS. 23a and b, DNA samples are sheared to a desired size then the Cap and random combinations of index sequences are symmetrically attached to the fragment ends via ligation. Following the final round of index ligation, a new adapter sequence containing an Illumina sequencing primer (SP1) adjacent to an optimized T7 RNA polymerase promoter is attached to the ends of the molecules via ligation as described above. In vitro transcription (IVT) via T7 RNA polymerase is then performed in order to amplify both ends of a given molecule. Following IVT, a primer containing a random nucleotide sequence of a set length (i.e., pentamer, hexamer, etc.) flanked by a different Illumina sequencing primer (SP2) is utilized as the primer in a reverse transcription reaction. Alternatively, RNA molecules may be trimmed to a desired size range and ligated to the Illumina sequencing primer (SP2) via standard techniques. Illumina P5 and P7 sites are then added to the cDNA via PCR using primers carrying Illumina P5-SP1 and P7-SP2 sequences.

Amplification of Ends Via Anchored PCR

As shown in FIG. 24, DNA samples are sheared to a desired size then the Cap and random combinations of index sequences are symmetrically attached to the fragment ends via ligation. Following the final round of index ligation, a new adapter containing an Illumina sequencing primer (SP1) adjacent to the Illumina P7 sequence is attached to the ends of the molecules via ligation as described above. The population of molecules is then incubated in the presence of Fragmentase or a cocktail of restriction endonucleases to liberate the ends of the molecules. Fragments are then tailed at the 3′ end using terminal transferase to attach a set number of specific nucleotides to the fragment ends, effectively creating a common priming sequence on the ends of all molecules. Alternatively, priming sequences may be ligated to the 3′ of the molecules using standard techniques. The fragments are then amplified via PCR using SP2-P7 and SP1-P5 primers where the SP1-P5 primer contains a tail complementary to the priming site attached in the previous step.

The invention will be further described by the following numbered paragraphs:

1. A method for labeling a nucleic acid at both its 5′ and 3′ ends with a unique label, comprising the steps of:
a) providing a pool of nucleic acids; and
b) sequentially end-labeling said nucleic acids with a random combination of n detectable oligonucleotide tags, each of said oligonucleotide tags optionally comprising a cohesive overhang of x base pairs in length, wherein each detectable oligonucleotide tag is randomly and independently selected from a number of detectable oligonucleotide tags that is less than the number of nucleic acids, and n is the number of oligonucleotides attached to an end of said nucleic acid,

wherein said method is performed in emulsion droplets, and

wherein each end-labeled nucleic acid is identically labeled at its 5′ and 3′ ends.

2. The method according to paragraph 1, wherein x is greater than about two base pairs.

3. The method according to paragraph 1, wherein x is from about two to about ten base pairs.

4. The method according to paragraph 1, wherein x is about four base pairs.

5. The method according to paragraph 1, wherein said detectable oligonucleotide tag is from about 10 to about 20 base pairs in length.

6. The method according to paragraph 3, wherein said oligonucleotide tag is selected from a tag in Table 1.

7. The method according to paragraph 1, wherein n is 2, 3, 4, 5, 6, 7, 8, 9, 10, 10², 10³, 10⁴, 10⁵, or 10⁶ or more detectable oligonucleotide tags.

8. A method, comprising:
sequentially attaching at least two detectable oligonucleotide tags to a 5′ and/or 3′ end a first nucleic acid, wherein each detectable oligonucleotide tag is randomly selected from a plurality of detectable oligonucleotide tags, thereby generating a second nucleic acid comprising the first nucleic acid attached at its 5′ and/or 3′ end with a unique combination of detectable oligonucleotide tags, wherein the plurality of second nucleic acids is generated using emulsion droplets.

9. The method of paragraph 8, wherein the first nucleic acid is a genomic DNA fragment.

10. The method of paragraph 9, wherein the second nucleic acid is a genomic DNA fragment attached to the unique combination of detectable oligonucleotide tags at its 5′ or 3′ end.

11. The method of paragraph 9, wherein the second nucleic acid is a genomic DNA fragment attached to the same unique combination of detectable oligonucleotide tags at its 5′ and 3′ end.

12. The method of paragraph 9, further comprising fragmenting the second nucleic acid.

13. The method of paragraph 8, wherein sequentially attaching the at least two detectable oligonucleotide tags to the first nucleic acid comprises ligation, polymerization, or a combination thereof.

14. A method, comprising:
(a) providing a population of library droplets comprising nucleic acids, wherein each droplet comprises a nucleic acid;
(b) fusing each individual library droplet with a single index droplet from a plurality of m₁ index droplets, each index droplet comprising a plurality of one unique detectable oligonucleotide tag;
(c) end-labeling the nucleic acid with the unique detectable oligonucleotide tag in a fused droplet;
(d) harvesting end-labeled nucleic acids from the fused droplets and generating another population of library droplets comprising end-labeled nucleic acids;
(e) repeating steps (b) to (d) n times to produce nucleic acids end-labeled with n unique detectable oligonucleotide tag, wherein the n unique detectable oligonucleotide tags generate an (m₁)(m₂)(m₃) . . . (mₙ) number of combinations that is greater than the number of starting nucleic acids; and
(f) amplifying the end-labeled nucleic acid formed in step (e).

15. The method of any one of paragraph 14, wherein end-labeling comprises ligation of the unique oligonucleotide tag with the nucleic acid.

16. The method of paragraph 15, wherein the unique oligonucleotide tag is double-stranded.

17. The method of according to paragraph 14, further comprising phosphorylating the nucleic acids between steps (b) and (c).

18. The method of according to paragraph 14, wherein end-labeling comprises a polymerase-mediated fill-in reaction.

19. The method of paragraph 18, wherein the polymerase-mediated fill-in reaction comprises:
(a) producing a single-stranded cohesive overhang on the nucleic acid, wherein the cohesive overhang is complementary to one end of the unique detectable oligonucleotide tag;
(b) annealing the complementary end of the unique oligonucleotide tag to the single-stranded cohesive overhang such that at least one nucleotide of the unique detectable oligonucleotide tag is not annealed to the nucleic acid, producing a unique detectable oligonucleotide tag cohesive overhang; and
(c) extending the single-stranded cohesive overhang of (a) using a polymerase and nucleotides complementary to the unique detectable oligonucleotide tag cohesive overhang to produce a double-stranded unique detectable oligonucleotide tag.

20. The method of paragraph 19, wherein the single-stranded cohesive overhang on the nucleic acid is produced by a USER enzyme.

21. The method of according to paragraph 19, wherein the unique detectable oligonucleotide tag is single-stranded.

22. The method of according to paragraph 19, wherein an oligonuclcotide adapter is added to the nucleic acids before labeling with the unique detectable oligonucleotide tags.

23. The method of paragraph 20, wherein the adapter comprises biotin.

24. The method of paragraph 20, wherein the adapter comprises a thymidine tail cohesive overhang.

25. The method of paragraph 19, wherein labeling occurs at the 5′ and 3′ ends of the nucleic acid.

26. The method of paragraph 19, wherein labeling occurs at the 5′ or the 3′ end of the nucleic acid.

27. A labeled nucleic acid obtainable by the method of paragraph 1.

28. The method of paragraph 15, wherein amplification step (f) comprises the steps of:
(i) attaching an adapter comprising a first sequencing primer to said nucleic acid;
(ii) incubating said nucleic acid in the presence of a transposome comprising a second sequencing primer; and
(iii) performing PCR amplification so as to amplify the ends of said nucleic acid.

29. The method of paragraph 14, wherein amplification step (f) comprises the steps of:
(i) attaching an adapter comprising a first sequencing primer to said nucleic acid;
(ii) performing in vitro transcription using a RNA polymerase;
(iii) performing a reverse transcription using a primer comprising a random nucleotide sequence of a given length flanked by a second sequencing primer or performing a reverse transcription using a primer comprising a nucleotide sequence attached to the 3′ end of the nucleic acid; and
(iv) performing PCR amplification so as to amplify the ends of said nucleic acid.

30. The method of paragraph 14, wherein amplification step (f) comprises the steps of:
(i) attaching an adapter comprising a first sequencing primer to said nucleic acid;
(ii) incubating said nucleic acid in fragmentase or a combination of one or more restriction endonucleases so as to liberate the ends of said nucleic acid thereby forming fragments;
(iii) attaching a given number of specific nucleotides to the ends of said fragments; and
(iv) performing PCR amplification on the fragments formed in step (iii) using a second sequencing primer.

It is to be understood that the invention is not limited to the particular embodiments of the invention described above, as variations of the particular embodiments may be made and still fall within the scope of the appended claims.

What is claimed is:

1. A method for labeling a nucleic acid at both its 5′ and 3′ ends with a unique label, comprising the steps of:
a) providing a pool of nucleic acids; and
b) sequentially end-labeling said nucleic acids with a random combination of n detectable oligonucleotide tags, each of said oligonucleotide tags optionally comprising a cohesive overhang of x base pairs in length, wherein each detectable oligonucleotide tag is randomly and independently selected from a number of detectable oligonucleotide tags that is less than the number of nucleic acids, and n is the number of oligonucleotides attached to an end of said nucleic acid,
wherein said method is performed in emulsion droplets, and
wherein each end-labeled nucleic acid is identically labeled at its 5′ and 3′ ends. nucleic acid

2. The method according to claim 1, wherein x is greater than about two base pairs.

3. The method according to claim 1, wherein x is from about two to about ten base pairs.

4. The method according to claim 1, wherein x is about four base pairs.

5. The method according to claim 1, wherein said detectable oligonucleotide tag is from about 10 to about 20 base pairs in length.

6. The method according to claim 3, wherein said oligonucleotide tag is selected from a tag in Table 1.

7. The method according to claim 1, wherein n is 2, 3, 4, 5, 6, 7, 8, 9, 10, 10², 10³, 10⁴, 10⁵, or 10⁶ or more detectable oligonucleotide tags.

8. A method, comprising:
sequentially attaching at least two detectable oligonucleotide tags to a 5′ and/or 3′ end a first nucleic acid, wherein each detectable oligonucleotide tag is randomly selected from a plurality of detectable oligonucleotide tags, thereby generating a second nucleic acid comprising the first nucleic acid attached at its 5′ and/or 3′ end with a unique combination of detectable oligonucleotide tags, wherein the plurality of second nucleic acids is generated using emulsion droplets.

9. The method of claim 8, wherein the first nucleic acid is a genomic DNA fragment.

10. The method of claim 9, wherein the second nucleic acid is a genomic DNA fragment attached to the unique combination of detectable oligonucleotide tags at its 5′ or 3′ end.

11. The method of claim 9, wherein the second nucleic acid is a genomic DNA fragment attached to the same unique combination of detectable oligonucleotide tags at its 5′ and 3′ end.

12. The method of claim 9, further comprising fragmenting the second nucleic acid.

13. The method of claim 8, wherein sequentially attaching the at least two detectable oligonucleotide tags to the first nucleic acid comprises ligation, polymerization, or a combination thereof.

14. A method, comprising:
(a) providing a population of library droplets comprising nucleic acids, wherein each droplet comprises a nucleic acid;
(b) fusing each individual library droplet with a single index droplet from a plurality of m₁ index droplets, each index droplet comprising a plurality of one unique detectable oligonucleotide tag;
(c) end-labeling the nucleic acid with the unique detectable oligonucleotide tag in a fused droplet;
(d) harvesting end-labeled nucleic acids from the fused droplets and generating another population of library droplets comprising end-labeled nucleic acids;
(e) repeating steps (b) to (d) n times to produce nucleic acids end-labeled with n unique detectable oligonucleotide tag, wherein the n unique detectable oligonucleotide tags generate an (m₁)(m₂)(m₃) . . . (mₙ) number of combinations that is greater than the number of starting nucleic acids; and
(f) amplifying the end-labeled nucleic acid formed in step (e).

15. The method of any one of claim 14, wherein end-labeling comprises ligation of the unique oligonucleotide tag with the nucleic acid.

16. The method of claim 15, wherein the unique oligonucleotide tag is double-stranded.

17. The method of according to claim 14, further comprising phosphorylating the nucleic acids between steps (b) and (c).

18. The method of according to claim 14, wherein end-labeling comprises a polymerase-mediated fill-in reaction.

19. The method of claim 18, wherein the polymerase-mediated fill-in reaction comprises:
(a) producing a single-stranded cohesive overhang on the nucleic acid, wherein the cohesive overhang is complementary to one end of the unique detectable oligonucleotide tag;
(b) annealing the complementary end of the unique oligonucleotide tag to the single-stranded cohesive overhang such that at least one nucleotide of the unique detectable oligonucleotide tag is not annealed to the nucleic acid, producing a unique detectable oligonucleotide tag cohesive overhang; and
(c) extending the single-stranded cohesive overhang of (a) using a polymerase and nucleotides complementary to the unique detectable oligonucleotide tag cohesive overhang to produce a double-stranded unique detectable oligonucleotide tag.

20. The method of claim 19, wherein the single-stranded cohesive overhang on the nucleic acid is produced by a USER enzyme.

21. The method of according to claim 19, wherein the unique detectable oligonucleotide tag is single-stranded.

22. The method of according to claim 19, wherein an oligonucleotide adapter is added to the nucleic acids before labeling with the unique detectable oligonucleotide tags.

23. The method of claim 20, wherein the adapter comprises biotin.

24. The method of claim 20, wherein the adapter comprises a thymidine tail cohesive overhang.

25. The method of claim 19, wherein labeling occurs at the 5′ and 3′ ends of the nucleic acid.

26. The method of claim 19, wherein labeling occurs at the 5′ or the 3′ end of the nucleic acid.

27. The method of claim 15, wherein amplification step (f) comprises the steps of:
(i) attaching an adapter comprising a first sequencing primer to said nucleic acid;
(ii) incubating said nucleic acid in the presence of a transposome comprising a second sequencing primer; and
(iii) performing PCR amplification so as to amplify the ends of said nucleic acid.

28. The method of claim 14, wherein amplification step (f) comprises the steps of:
(i) attaching an adapter comprising a first sequencing primer to said nucleic acid;
(ii) performing in vitro transcription using a RNA polymerase;
(iii) performing a reverse transcription using a primer comprising a random nucleotide sequence of a given length flanked by a second sequencing primer or performing a reverse transcription using a primer comprising a nucleotide sequence attached to the 3′ end of the nucleic acid; and
(iv) performing PCR amplification so as to amplify the ends of said nucleic acid.

29. The method of claim 14, wherein amplification step (f) comprises the steps of:
(i) attaching an adapter comprising a first sequencing primer to said nucleic acid;
(ii) incubating said nucleic acid in fragmentase or a combination of one or more restriction endonucleases so as to liberate the ends of said nucleic acid thereby forming fragments;
(iii) attaching a given number of specific nucleotides to the ends of said fragments; and
(iv) performing PCR amplification on the fragments formed in step (iii) using a second sequencing primer.
